SEQUENCE LISTING (142 sequences)

SEQ ID NO: 1 - protein, 10 aa, Homo sapiens
NTENYILWGY

SEQ ID NO: 2 - protein, 11 aa, Homo sapiens
MKFGNQVSGLF

SEQ ID NO: 3 - protein, 10 aa, Homo sapiens
RLQHEPPHPV

SEQ ID NO: 4 - protein, 9 aa, Homo sapiens
LPMWKALLF

SEQ ID NO: 5 - protein, 9 aa, Homo sapiens
RPARPPAGL

SEQ ID NO: 6 - protein, 10 aa, Homo sapiens
FAYPNQKVTF

SEQ ID NO: 7 - protein, 9 aa, Homo sapiens
GQAHPQGSF

SEQ ID NO: 8 - protein, 10 aa, Homo sapiens
YSDQKPPYSY

SEQ ID NO: 9 - protein, 9 aa, Homo sapiens
KLAQIIRQV

SEQ ID NO: 10 - protein, 9 aa, Homo sapiens
GEIKTFSDL

SEQ ID NO: 11 - protein, 9 aa, Homo sapiens
GKLDNTNEY

SEQ ID NO: 12 - protein, 9 aa, Homo sapiens
AKKKENITY

SEQ ID NO: 13 - protein, 9 aa, Homo sapiens
SAQGKPTYF

SEQ ID NO: 14 - protein, 9 aa, Homo sapiens
EEEIHSPTL

SEQ ID NO: 15 - protein, 10 aa, Homo sapiens
SKLRSTGQSF

SEQ ID NO: 16 - protein, 9 aa, Homo sapiens
NTLSESYIY

SEQ ID NO: 17 - protein, 10 aa, Homo sapiens
QPLPQPLELW

SEQ ID NO: 18 - protein, 10 aa, Homo sapiens
RTDTGKRVLY

SEQ ID NO: 19 - protein, 10 aa, Homo sapiens
LPSGETIAKW

SEQ ID NO: 20 - protein, 9 aa, Homo sapiens
KLDSYIIPY

SEQ ID NO: 21 - protein, 8 aa, Homo sapiens
ISMCDLVY

SEQ ID NO: 22 - protein, 9 aa, Homo sapiens
YKRMKLDSY

SEQ ID NO: 23 - protein, 10 aa, Homo sapiens
QPLPEPLQLW

SEQ ID NO: 24 - protein, 8 aa, Homo sapiens
IPMKIYLW

SEQ ID NO: 25 - protein, 9 aa, Homo sapiens
SQSSLMLYL

SEQ ID NO: 26 - protein, 9 aa, Homo sapiens
ALYPQPPTV

SEQ ID NO: 27 - protein, 10 aa, Homo sapiens
YTPFPSYGHY

SEQ ID NO: 28 - protein, 11 aa, Homo sapiens
FTEEDLHFVLY

SEQ ID NO: 29 - protein, 9 aa, Homo sapiens
GQITHNTSF

SEQ ID NO: 30 - protein, 9 aa, Homo sapiens
VTLSTYFHV

SEQ ID NO: 31 - protein, 9 aa, Homo sapiens
GQFDRPAGV

SEQ ID NO: 32 - protein, 9 aa, Homo sapiens
VSDQQNGTY

SEQ ID NO: 33 - protein, 11 aa, Homo sapiens
KHFDSPRGVAF

SEQ ID NO: 34 - protein, 9 aa, Homo sapiens
GEHLVSVTL

SEQ ID NO: 35 - protein, 9 aa, Homo sapiens
YSIYPMRNL

SEQ ID NO: 36 - protein, 8 aa, Homo sapiens
SQNSPIRY

SEQ ID NO: 37 - protein, 10 aa, Homo sapiens
FHSQNSPIRY

SEQ ID NO: 38 - protein, 9 aa, Homo sapiens
VAKPPGTAF

SEQ ID NO: 39 - protein, 11 aa, Homo sapiens
SLLGSSEILEV

SEQ ID NO: 40 - protein, 9 aa, Homo sapiens
TASDLNLKV

SEQ ID NO: 41 - protein, 11 aa, Homo sapiens
VLMDEGAVLTL

SEQ ID NO: 42 - protein, 9 aa, Homo sapiens
TEFQQIINL

SEQ ID NO: 43 - protein, 9 aa, Homo sapiens
KVLEHVVRV

SEQ ID NO: 44 - protein, 10 aa, Homo sapiens
GVYDGREHTV

SEQ ID NO: 45 - protein, 10 aa, Homo sapiens
GLYDGREHSV

SEQ ID NO: 46 - protein, 10 aa, Homo sapiens
NPIGDTGVKF

SEQ ID NO: 47 - protein, 10 aa, Homo sapiens
GKFQGLIEKF

SEQ ID NO: 48 - protein, 10 aa, Homo sapiens
KQMENDIQLY

SEQ ID NO: 49 - protein, 9 aa, Homo sapiens
ASTPASSEL

SEQ ID NO: 50 - protein, 10 aa, Homo sapiens
RLWNETVELF

SEQ ID NO: 51 - protein, 9 aa, Homo sapiens
FGDGKFSEV

SEQ ID NO: 52 - protein, 11 aa, Homo sapiens
SEVSADKLVAL

SEQ ID NO: 53 - protein, 9 aa, Homo sapiens
AMYHALEKA

SEQ ID NO: 54 - protein, 9 aa, Homo sapiens
FAYPNQKDF

SEQ ID NO: 55 - protein, 11 aa, Homo sapiens
ALLEGVNTVVV

SEQ ID NO: 56 - protein, 10 aa, Homo sapiens
SETHPPEVAL

SEQ ID NO: 57 - protein, 10 aa, Homo sapiens
SPQEASGVRW

SEQ ID NO: 58 - protein, 9 aa, Homo sapiens
YLLNCHLLI

SEQ ID NO: 59 - protein, 9 aa, Homo sapiens
GQFLVKSGY

SEQ ID NO: 60 - protein, 11 aa, Homo sapiens
QTELNNSKQEY

SEQ ID NO: 61 - protein, 9 aa, Homo sapiens
MAWNGILHL

SEQ ID NO: 62 - protein, 10 aa, Homo sapiens
HPLPGLILEW

SEQ ID NO: 63 - protein, 9 aa, Homo sapiens
TPSPIIQQL

SEQ ID NO: 64 - protein, 11 aa, Homo sapiens
FLLPGVLLSEA

SEQ ID NO: 65 - protein, 9 aa, Homo sapiens
SANVSKVSF

SEQ ID NO: 66 - protein, 9 aa, Homo sapiens
LALGNTKEL

SEQ ID NO: 67 - protein, 9 aa, Homo sapiens
KQFEGTVEI

SEQ ID NO: 68 - protein, 9 aa, Homo sapiens
KLQEKIQEL

SEQ ID NO: 69 - protein, 9 aa, Homo sapiens
KMSELQTYV

SEQ ID NO: 70 - protein, 9 aa, Homo sapiens
RLWNETVEL

SEQ ID NO: 71 - protein, 9 aa, Homo sapiens
ALLEGVNTV

SEQ ID NO: 72 - protein, 9 aa, Homo sapiens
GQKENVVVY

SEQ ID NO: 73 - protein, 17 aa, Homo sapiens
CASSQPPAAGAKNIQYF

SEQ ID NO: 74 - protein, 13 aa, Homo sapiens
CASSITGGEKLFF

SEQ ID NO: 75 - protein, 13 aa, Homo sapiens
CASSKWGSYGYTF

SEQ ID NO: 76 - protein, 14 aa, Homo sapiens
CASSLPDTTYEQYF

SEQ ID NO: 77 - protein, 14 aa, Homo sapiens
CASSYRTGSAEAFF

SEQ ID NO: 78 - protein, 13 aa, Homo sapiens
CASSLGGAYEQYF

SEQ ID NO: 79 - protein, 13 aa, Homo sapiens
CASSYPQGGEQFF

SEQ ID NO: 80 - protein, 15 aa, Homo sapiens
CATSRGTGVNTEAFF

SEQ ID NO: 81 - protein, 15 aa, Homo sapiens
CASSSRTDTYSPLHF

SEQ ID NO: 82 - protein, 14 aa, Homo sapiens
CASSLGRWNSPLHF

SEQ ID NO: 83 - protein, 13 aa, Homo sapiens
CASSGPGDQPQHF

SEQ ID NO: 84 - protein, 18 aa, Homo sapiens
CASSEWGSRAGGGDTQYF

SEQ ID NO: 85 - protein, 13 aa, Homo sapiens
CASSMGRGHEQYF

SEQ ID NO: 86 - protein, 15 aa, Homo sapiens
CASSHFGRRGTEAFF

SEQ ID NO: 87 - protein, 15 aa, Homo sapiens
CASSSHLPYPYEQYF

SEQ ID NO: 88 - DNA, 30 bp, Homo sapiens
  source 1..30: mol_type = other DNA
aacacagaaaattatatactctggggttac

SEQ ID NO: 89 - DNA, 33 bp, Homo sapiens
  source 1..33: mol_type = other DNA
atgaaatttggtaatcaggtaagcggactcttc

SEQ ID NO: 90 - DNA, 30 bp, Homo sapiens
  source 1..30: mol_type = other DNA
agattacagcatgagccaccacaccctgtc

SEQ ID NO: 91 - DNA, 27 bp, Homo sapiens
  source 1..27: mol_type = other DNA
ctgcccatgtggaaggctctgctgttt

SEQ ID NO: 92 - DNA, 27 bp, Homo sapiens
  source 1..27: mol_type = other DNA
cgccctgcacggccgccagccggcctc

SEQ ID NO: 93 - DNA, 30 bp, Homo sapiens
  source 1..30: mol_type = other DNA
tttgcctacccaaatcaaaaggtaactttt

SEQ ID NO: 94 - DNA, 27 bp, Homo sapiens
  source 1..27: mol_type = other DNA
ggtcaagctcacccccaaggcagcttc

SEQ ID NO: 95 - DNA, 30 bp, Homo sapiens
  source 1..30: mol_type = other DNA
tacagcgaccagaagccgccctactcgtac

SEQ ID NO: 96 - DNA, 27 bp, Homo sapiens
  source 1..27: mol_type = other DNA
aagctggcccagatcatccgtcaggtc

SEQ ID NO: 97 - DNA, 27 bp, Homo sapiens
  source 1..27: mol_type = other DNA
ggtgaaataaagacattttcagatctg

SEQ ID NO: 98 - DNA, 27 bp, Homo sapiens
  source 1..27: mol_type = other DNA
ggtaaattagacaacacgaacgaatac

SEQ ID NO: 99 - DNA, 27 bp, Homo sapiens
  source 1..27: mol_type = other DNA
gcaaagaaaaaggaaaatatcacctat

SEQ ID NO: 100 - DNA, 27 bp, Homo sapiens
  source 1..27: mol_type = other DNA
agtgcacagggaaagccaacctacttt

SEQ ID NO: 101 - DNA, 27 bp, Homo sapiens
  source 1..27: mol_type = other DNA
gaagaagaaatccacagccctaccctg

SEQ ID NO: 102 - DNA, 30 bp, Homo sapiens
  source 1..30: mol_type = other DNA
tccaaactgagaagcactggacagtcattt

SEQ ID NO: 103 - DNA, 27 bp, Homo sapiens
  source 1..27: mol_type = other DNA
aacaccctgagtgaatcttacatttat

SEQ ID NO: 104 - DNA, 30 bp, Homo sapiens
  source 1..30: mol_type = other DNA
cagccacttccacagcccctggaactctgg

SEQ ID NO: 105 - DNA, 30 bp, Homo sapiens
  source 1..30: mol_type = other DNA
aggactgacacaggtaagagagtcttgtat

SEQ ID NO: 106 - DNA, 30 bp, Homo sapiens
  source 1..30: mol_type = other DNA
ttgccaagcggtgagactatcgccaagtgg

SEQ ID NO: 107 - DNA, 27 bp, Homo sapiens
  source 1..27: mol_type = other DNA
aagttggactcttacattataccatat

SEQ ID NO: 108 - DNA, 24 bp, Homo sapiens
  source 1..24: mol_type = other DNA
atttctatgtgtgatttggtttat

SEQ ID NO: 109 - DNA, 27 bp, Homo sapiens
  source 1..27: mol_type = other DNA
tacaaaagaatgaagttggactcttac

SEQ ID NO: 110 - DNA, 30 bp, Homo sapiens
  source 1..30: mol_type = other DNA
cagccactcccagagcccctgcaactctgg

SEQ ID NO: 111 - DNA, 24 bp, Homo sapiens
  source 1..24: mol_type = genomic DNA
atccccatgaaaatatatctctgg

SEQ ID NO: 112 - DNA, 27 bp, Homo sapiens
  source 1..27: mol_type = other DNA
tcccagagctcactaatgctatattta

SEQ ID NO: 113 - DNA, 27 bp, Homo sapiens
  source 1..27: mol_type = other DNA
gcgctgtatcctcagccgcccactgtg

SEQ ID NO: 114 - DNA, 30 bp, Homo sapiens
  source 1..30: mol_type = other DNA
tacacgcctttcccgtcctatggacactac

SEQ ID NO: 115 - DNA, 33 bp, Homo sapiens
  source 1..33: mol_type = other DNA
ttcacggaggaggacctgcacttcgttctgtac

SEQ ID NO: 116 - DNA, 27 bp, Homo sapiens
  source 1..27: mol_type = other DNA
gggcaaatcacacataacacttcattc

SEQ ID NO: 117 - DNA, 27 bp, Homo sapiens
  source 1..27: mol_type = other DNA
gtcaccttgagtacttatttccatgtg

SEQ ID NO: 118 - DNA, 27 bp, Homo sapiens
  source 1..27: mol_type = other DNA
gggcagttcgaccgaccagccggcgtg

SEQ ID NO: 119 - DNA, 27 bp, Homo sapiens
  source 1..27: mol_type = other DNA
gtgagtgatcagcagaatgggacatac

SEQ ID NO: 120 - DNA, 33 bp, Homo sapiens
  source 1..33: mol_type = other DNA
aagcactttgactccccacggggtgtggccttc

SEQ ID NO: 121 - DNA, 27 bp, Homo sapiens
  source 1..27: mol_type = other DNA
ggtgagcacctggtatctgtgacactg

SEQ ID NO: 122 - DNA, 27 bp, Homo sapiens
  source 1..27: mol_type = other DNA
tactccatctaccccatgcgcaacctc

SEQ ID NO: 123 - DNA, 24 bp, Homo sapiens
  source 1..24: mol_type = other DNA
tctcaaaactctcctataaggtat

SEQ ID NO: 124 - DNA, 30 bp, Homo sapiens
  source 1..30: mol_type = other DNA
tttcattctcaaaactctcctataaggtat

SEQ ID NO: 125 - DNA, 27 bp, Homo sapiens
  source 1..27: mol_type = other DNA
gtagccaaacccccaggaactgctttc

SEQ ID NO: 126 - DNA, 33 bp, Homo sapiens
  source 1..33: mol_type = other DNA
tccctgcttggcagcagtgaaattttggaggtc

SEQ ID NO: 127 - DNA, 30 bp, Homo sapiens
  source 1..30: mol_type = other DNA
gggaagttccagggtctgattgagaagttt

SEQ ID NO: 128 - DNA, 30 bp, Homo sapiens
  source 1..30: mol_type = other DNA
aagcaaatggaaaatgatattcagttatat

SEQ ID NO: 129 - DNA, 27 bp, Homo sapiens
  source 1..27: mol_type = other DNA
gcctccactccagcctcatcagagtta

SEQ ID NO: 130 - DNA, 30 bp, Homo sapiens
  source 1..30: mol_type = other DNA
aggctgtggaatgagaccgtggagcttttt

SEQ ID NO: 131 - DNA, 27 bp, Homo sapiens
  source 1..27: mol_type = other DNA
tttggcgatggcaagttctccgaggtc

SEQ ID NO: 132 - DNA, 33 bp, Homo sapiens
  source 1..33: mol_type = other DNA
tccgaggtctctgcagacaaactggtggcactg

SEQ ID NO: 133 - DNA, 27 bp, Homo sapiens
  source 1..27: mol_type = other DNA
gccatgtaccatgctctggagaaagcc

SEQ ID NO: 134 - DNA, 27 bp, Homo sapiens
  source 1..27: mol_type = other DNA
tttgcctacccaaatcaaaaggatttt

SEQ ID NO: 135 - DNA, 33 bp, Homo sapiens
  source 1..33: mol_type = other DNA
gcgctccttgagggagttaatacagttgtggtg

SEQ ID NO: 136 - DNA, 30 bp, Homo sapiens
  source 1..30: mol_type = other DNA
tctgagacacatcctcctgaagtggctctt

SEQ ID NO: 137 - DNA, 30 bp, Homo sapiens
  source 1..30: mol_type = other DNA
tctccacaagaggcctctggtgtcaggtgg

SEQ ID NO: 138 - DNA, 27 bp, Homo sapiens
  source 1..27: mol_type = other DNA
taccttctaaactgccacctgttaatc

SEQ ID NO: 139 - DNA, 27 bp, Homo sapiens
  source 1..27: mol_type = other DNA
ggccagttcttggtcaaatccggctac

SEQ ID NO: 140 - DNA, 33 bp, Homo sapiens
  source 1..33: mol_type = other DNA
cagacagagctgaataattcaaagcaagaatat

SEQ ID NO: 141 - DNA, 27 bp, Homo sapiens
  source 1..27: mol_type = other DNA
atggcatggaatggaatcctacacctc

SEQ ID NO: 142 - DNA, 30 bp, Homo sapiens
  source 1..30: mol_type = genomic DNA
catcctttaccaggcttgatattagaatgg

(19) United States
(12) Patent Application Publication
(10) Pub. No.: US 2024/0382590 A1
(43) Pub. Date: Nov. 21, 2024

(54) NOVEL TUMOR-SPECIFIC ANTIGENS FOR CANCER STEM CELLS AND USES THEREOF

(71) Applicant: UNIVERSITÉ DE MONTRÉAL, Montréal, Québec (CA)

(72) Inventors: Claude PERREAULT, Montréal, Québec (CA); Pierre THIBAULT, Montréal, Québec (CA); Marie-pierre HARDY, Terrebonne, Québec (CA); Anca APAVALOAEI, Montréal, Québec (CA)

(21) Appl. No.: 18/579,831

(22) PCT Filed: Jul. 7, 2022
(86) PCT No.: PCT/CA2022/051068

§ 371 Date: Jan. 16, 2024

Related U.S. Application Data
(60) Provisional application No. 63/203,320, filed on Jul. 16, 2021.

(51) Int. Cl. A61K 39/00; A61K 35/17; A61K 38/00; C07K 7/06; C12N 5/0783

(52) U.S. Cl. CPC A61K 39/464401; A61K 35/17; A61K 39/4611; C07K 7/06; C12N 5/0636; A61K 38/00; A61K 2039/55555; A61K 2039/572; C12N 2510/00

(57) ABSTRACT

Cancer stem cells (CSCs) are a subpopulation of tumor cells that can drive tumor initiation and can cause relapses. These cells are seen as drivers of tumor establishment and growth, often correlated to aggressive, heterogeneous and therapy-resistant tumors. Novel tumor-specific antigens (TSAs) and tumor-associated antigens (TAAs) specifically expressed by CSCs are described herein. Most of the TSAs described herein derives from aberrantly expressed unmutated genomic sequences, such as intronic and intergenic sequences, which are not expressed in normal tissues. Nucleic acids, compositions, cells, antibodies and vaccines derived from these TSAs are described. The use of the TSAs, nucleic acids, compositions, antibodies, cells and vaccines for the treatment of cancer, and more particularly cancers associated with the presence of CSCs such as poorly differentiated cancers, is also described.

### CROSS REFERENCE TO RELATED APPLICATIONS

The present application claims the benefit of U.S. provisional patent application No. 63/203,320, filed on Jul. 16, 2021, which is incorporated herein by reference.

### TECHNICAL FIELD

The present invention generally relates to the field of oncology, and more particularly to the treatment of cancers associated with cancer stem cells.

### BACKGROUND ART

According to a World Health Organization (WHO) report, 8.2 million patients died from cancer in 2012. Cancer is therefore a continuously growing health problem in both developing and developed countries. It has also been estimated that the number of annual cancer cases will increase within the next two decades. The common general treatments for cancer are surgery, endocrine therapy, chemotherapy, immunotherapy and radiotherapy.

Because of all these treatments, the incidence rate of cancer has been stable in women and has declined slightly in men during recent years (2006-2015), and the cancer death rate (2007-2016) also declined. However, traditional cancer treatment methods are effective only for some malignant tumors. The main reasons for the failure of cancer treatment are metastasis, recurrence, heterogeneity, resistance to chemotherapy and radiotherapy, and avoidance of immunological surveillance.

Cancer stem cells (CSCs) or tumor-initiating cells (TICs) are a subpopulation of tumor cells that can drive tumor initiation and can cause relapses. These cells are seen as drivers of tumor establishment and growth, often correlated to aggressive, heterogeneous and therapy-resistant tumors.

There is thus a need for novel therapeutic approaches for the treatment of cancers, and notably approaches that target CSCs.

The present description refers to a number of documents, the content of which is herein incorporated by reference in their entirety.

### SUMMARY

The present disclosure provides the following items 1 to 57:

1. A cancer stem cell (CSC) tumor antigen peptide (TAP) comprising of one of the following amino acid sequences:

| Peptide | SEQ | Peptide | SEQ |
|---|---|---|---|
| sequence | ID NO: | sequence | ID NO: |
| NTENYILWGY | 1 | ISMCDLVY | 21 |
| MKFGNQVSGLF | 2 | YKRMKLDSY | 22 |
| RLQHEPPHPV | 3 | QPLPEPLQLW | 23 |
| LPMWKALLF | 4 | IPMKIYLW) | 24 |
| RPARPPAGL | 5 | SQSSLMLYL | 25 |
| FAYPNQKVTF | 6 | ALYPQPPTV | 26 |
| GQAHPQGSF | 7 | YTPFPSYGHY | 27 |
| YSDQKPPYSY | 8 | FTEEDLHFVLY | 28 |
| KLAQIIRQV | 9 | GQITHNTSF | 29 |
| GEIKTFSDL | 10 | VTLSTYFHV | 30 |
| GKLDNTNEY | 11 | GQFDRPAGV | 31 |
| AKKKENITY | 12 | VSDQQNGTY | 32 |
| SAQGKPTYF | 13 | KHFDSPRGVAF | 33 |
| EEEIHSPTL | 14 | GEHLVSVTL | 34 |
| SKLRSTGQSF | 15 | YSIYPMRNL | 35 |
| NTLSESYIY | 16 | SQNSPIRY | 36 |
| QPLPQPLELW | 17 | FHSQNSPIRY | 37 |
| RTDTGKRVLY | 18 | VAKPPGTAF | 38 |
| LPSGETIAKW | 19 | SLLGSSEILEV | 39 |
| KLDSYIIPY | 20 | | |
| Peptide | SEQ | Peptide | SEQ |
| sequence | ID NO: | sequence | ID NO: |
| GKFQGLIEKF | 47 | ALLEGVNTVVV | 55 |
| KQMENDIQLY | 48 | SETHPPEVAL | 56 |
| ASTPASSEL | 49 | SPQEASGVRW | 57 |
| RLWNETVELF | 50 | YLLNCHLLI | 58 |
| FGDGKFSEV | 51 | GQFLVKSGY | 59 |
| SEVSADKLVAL | 52 | QTELNNSKQEY | 60 |
| AMYHALEKA | 53 | MAWNGILHL | 61 |
| FAYPNQKDF | 54 | HPLPGLILEW | 62 |

or a nucleic acid encoding said CSC TAP.
2. The CSC TAP or nucleic acid of item 1, wherein said CSC TAP comprises one of the sequences defined in SEQ ID NO: 1-39.
3. The CSC TAP or nucleic acid of item 1 or 2, which binds to an HLA-A*01:01 molecule and comprises the sequence of SEQ ID NO: 1, 8, 16, 20, 21, 27, 28, 32, 37 or 60.
4. The CSC TAP or nucleic acid of item 1 or 2, which binds to an HLA-A*02:01 molecule and comprises the sequence of SEQ ID NO: 3, 6, 26, 30, 31, 39, 53, 55 or 58.
5. The CSC TAP or nucleic acid of item 1 or 2, which binds to an HLA-B*07:02 molecule and comprises the sequence of SEQ ID NO: 5.
6. The CSC TAP or nucleic acid of item 1 or 2, which binds to an HLA-B*15:03 molecule and comprises the sequence of SEQ ID NO: 2, 7, 11, 12, 15, 22, 29, 36, 38, 47, 48, or 59, preferably SEQ ID NO:2, 7, 11, 12, 15, 22, 29, 36 or 38.
7. The CSC TAP or nucleic acid of item 1 or 2, which binds to an HLA-B*40:01 molecule and comprises the sequence of SEQ ID NO: 10, 25, 34, 52 or 56.
8. The CSC TAP or nucleic acid of item 1 or 2, which binds to an HLA-B*53:01 molecule and comprises the sequence of SEQ ID NO: 4, 17, 19, 23, 24 or 57.
9. The CSC TAP or nucleic acid of item 1 or 2, which binds to an HLA-C*02:10 molecule and comprises the sequence of SEQ ID NO: 6, 54 or 61.
10. The CSC TAP or nucleic acid of item 1 or 2, which binds to an HLA-C*03:04 molecule and comprises the sequence of SEQ ID NO: 6, 35, 49 or 51.
11. The CSC TAP or nucleic acid of item 1 or 2, which binds to an HLA-C*04:01 molecule and comprises the sequence of SEQ ID NO: 13, 33 or 50.
12. The CSC TAP or nucleic acid of any one of items 1-11, which is encoded by a sequence located a non-protein coding region of the genome.
13. The CSC TAP or nucleic acid of item 12, wherein said non-protein coding region of the genome is an untranslated transcribed region (UTR).
14. The CSC TAP or nucleic acid of item 12, wherein said non-protein coding region of the genome is an intron.
15. The CSC TAP or nucleic acid of item 12, wherein said non-protein coding region of the genome is an intergenic region.
16. The CSC TAP or nucleic acid of item 12, wherein said non-protein coding region of the genome is a long non-coding RNAs.
17. The CSC TAP or nucleic acid of any one of items 1 to 16, which is a nucleic acid.
18. A combination comprising at least two of the CSC TAPs or nucleic acids defined in any one of items 1-16.
19. The CSC TAP or nucleic acid of any one of items 1 to 17, or the combination of claim 18, wherein the nucleic acid is an mRNA.
20. The CSC TAP or nucleic acid of any one of items 1 to 17, or the combination of claim 18, wherein the nucleic acid is a DNA.
21. The CSC TAP, nucleic acid or combination of any one of items 1 to 20, wherein the nucleic acid is a component of a viral vector.
22. A lipid vesicle or particle comprising the CSC TAP, nucleic acid or combination of any one of items 1 to 21.
23. The lipid vesicle or particle of item 22, wherein the lipid vesicle is a lipid nanoparticle (LNP).
24. The lipid vesicle or particle of item 22 or 23, which comprises a cationic lipid.
25. A composition comprising the CSC TAP, nucleic acid or combination of any one of items 1 to 21, or the lipid vesicle or particle of any one of items 22-24, and a pharmaceutically acceptable carrier.
26. A vaccine comprising the CSC TAP, nucleic acid or combination of any one of items 1 to 21, or the lipid vesicle or particle of any one of items 22-24, or the composition of item 25, and an adjuvant.
27. An isolated major histocompatibility complex (MHC) class I molecule comprising the CSC TAP of any one of items 1-16 in its peptide binding groove.
28. The isolated MHC class I molecule of item 27, which is in the form of a multimer.
29. The isolated MHC class I molecule of item 28, wherein said multimer is a tetramer.
30. An isolated cell comprising the CSC TAP, nucleic acid or combination of any one of items 1 to 21.
31. An isolated cell expressing at its surface major histocompatibility complex (MHC) class I molecules comprising the CSC TAP of any one of items 1-16 or the combination of item 18 in their peptide binding groove.
32. The cell of item 30 or 31, which is an antigen-presenting cell (APC).
33. The cell of item 32, wherein said APC is a dendritic cell.
34. A T-cell receptor (TCR) that specifically recognizes the isolated MHC class I molecule of any one of items 27-29 and/or MHC class I molecules expressed at the surface of the cell of any one of items 31-33.
35. An antibody or an antigen-binding fragment thereof that specifically binds to the isolated MHC class I molecule of any one of items 27-29 and/or MHC class I molecules expressed at the surface of the cell of any one of items 31-33.
36. The antibody or antigen-binding fragment thereof according to item 35, which is a bispecific antibody or antigen-binding fragment thereof.
37. The antibody or antigen-binding fragment thereof according to item 36, wherein the bispecific antibody or antigen-binding fragment thereof is a single-chain diabody (scDb).
38. The antibody or antigen-binding fragment thereof according to item 36 or 37, wherein the bispecific antibody or antigen-binding fragment thereof also specifically binds to a T cell signaling molecule.
39. The antibody or antigen-binding fragment thereof according to item 38, wherein the T cell signaling molecule is a CD3 chain.
40. An isolated cell expressing at its cell surface the TCR of item 34.
41. The isolated cell of item 40, which is a CD8⁺ T lymphocyte.
42. A cell population comprising at least 0.5% of the isolated cell as defined in item 40 or 41.
43. A method of treating cancer in a subject comprising administering to the subject an effective amount of: (i) the CSC TAP, nucleic acid or combination of any one of items 1 to 21; (ii) the lipid vesicle or particle of any one of items 22-24; (iii) the composition of item 25 (iv) the vaccine of item 26; (v) the cell or cell population of any one of items 30-33 and 40-42; or (vii) the antibody or antigen-binding fragment thereof of any one of items 35-39.
44. The method of item 43, wherein the cancer is leukemia (e.g., AML), brain cancer (e.g., glioblastoma), breast cancer, lung cancer, gastrointestinal cancer (e.g., colorectal cancer, gastric cancer, esophageal cancer), liver cancer (e.g., hepatocellular carcinoma), ovarian cancer, pancreatic cancer, prostate cancer, skin cancer (e.g., melanoma), head and neck cancer or myeloma (e.g., multiple myeloma).
45. The method of item 43 or 44, further comprising administering at least one additional antitumor agent or therapy to the subject.
46. The method of item 45, wherein said at least one additional antitumor agent or therapy is a chemotherapeutic agent, immunotherapy, an immune checkpoint inhibitor, radiotherapy or surgery.
47. The method of item 46, wherein said at least one additional antitumor agent or therapy comprises an inhibitor of CDK4/6, TGF-β and/or WNT-β-catenin.
48. Use of (i) the CSC TAP, nucleic acid or combination of any one of items 1 to 21; (ii) the lipid vesicle or particle of any one of items 22-24; (iii) the composition of item 25 (iv) the vaccine of item 26; (v) the cell or cell population of any one of items 30-33 and 40-42; or (vii) the antibody or antigen-binding fragment thereof of any one of items 35-39, for treating cancer in a subject, or for the manufacture of a medicament for treating cancer in a subject.
49. The use of item 48, wherein the cancer is leukemia (e.g., AML), brain cancer (e.g., glioblastoma), breast cancer, lung cancer, gastrointestinal cancer (e.g., colorectal cancer, gastric cancer, esophageal cancer), liver cancer (e.g., hepatocellular carcinoma), ovarian cancer, pancreatic cancer, prostate cancer, skin cancer (e.g., melanoma), head and neck cancer or myeloma (e.g., multiple myeloma).
50. The use of item 48 or 49, further comprising the use at least one additional antitumor agent or therapy to the subject.
51. The use of item 50, wherein said at least one additional antitumor agent or therapy is a chemotherapeutic agent, immunotherapy, an immune checkpoint inhibitor, radiotherapy or surgery.
52. The use of item 51, wherein said at least one additional antitumor agent or therapy comprises an inhibitor of CDK4/6, TGF-β and/or WNT-β-catenin.
53. An agent for use in treating cancer in a subject, wherein the agent is: (i) the CSC TAP, nucleic acid or combination of any one of items 1 to 21; (ii) the lipid vesicle or particle of any one of items 22-24; (iii) the composition of item 25 (iv) the vaccine of item 26; (v) the cell or cell population of any one of items 30-33 and 40-42; or (vii) the antibody or antigen-binding fragment thereof of any one of items 35-39.
54. The agent for use of item 53, wherein the cancer is leukemia (e.g., AML), brain cancer (e.g., glioblastoma), breast cancer, lung cancer, gastrointestinal cancer (e.g., colorectal cancer, gastric cancer, esophageal cancer), liver cancer (e.g., hepatocellular carcinoma), ovarian cancer, pancreatic cancer, prostate cancer, skin cancer (e.g., melanoma), head and neck cancer or myeloma (e.g., multiple myeloma).
55. The agent for use of item 53 or 54, further comprising the use at least one additional antitumor agent or therapy to the subject.
56. The agent for use of item 55, wherein said at least one additional antitumor agent or therapy is a chemotherapeutic agent, immunotherapy, an immune checkpoint inhibitor, radiotherapy or surgery.
57. The agent for use of item 56, wherein said at least one additional antitumor agent or therapy comprises an inhibitor of CDK4/6, TGF-β and/or WNT-β-catenin.

Other objects, advantages and features of the present disclosure will become more apparent upon reading of the following non-restrictive description of specific embodiments thereof, given by way of example only with reference to the accompanying drawings.

### BRIEF DESCRIPTION OF DRAWINGS

In the appended drawings:

FIGS. 1A and B depict the approach used for the MS-based identification of paMAPs using human iPSCs. FIG. 1A: Workflow for paMAP identification using iPSCs, based on the proteogenomic approach from (Laumont et al., 2018). pMHC-IP, peptide-MHC I immunoprecipitation; MAP, MHC I-associated peptide; TEC, thymic epithelial cells; LC-MS/MS, liquid chromatography with tandem mass spectrometry; FDR, false discovery rate; RPHM, reads per hundred million. FIG. 1B: Total number of MAPs identified per iPSC sample before MAP annotation.

FIGS. 2A-H show that the immunopeptidome of iPSCs reflects their pluripotency state. FIG. 2A: Heatmap showing the mean RNA expression [log 10(RPHM+1)] of paMAPs and saMAPs in healthy tissues from the GTEx consortium (n=5-150, depending on sample availability) and in mTECs (n=11). Boxed: tissues with expression>8.55 RPHM in >25% of samples. FIG. 2B: Heatmap showing the mean RNA expression [log 10(RPHM+1)] of paMAPs and saMAPs in PSCs (from this study and from (Churko et al., 2017)) and ASCs (healthy sorted primary adult stem cells, normal hematopoietic precursors (prec.) or cord blood samples). Boxed: mean expression across samples>8.55 RPHM. The number of samples in each sample group is in parentheses. MSC, mesenchymal stem cells. FIG. 2C: Pie chart displaying the percentage of paMAP-source genes corresponding to each biotype and the class of the ERE overlapping at the respective paMAP-coding region, if applicable. FIGS. 2D-E: Top: Number of saMAPs (FIG. 2D) or paMAPs (FIG. 2E) derived from each source gene. Bottom: Reactome pathways significantly enriched in saMAP (FIG. 2D) or paMAP (FIG. 2E)-source genes. FIG. 2F: Boxplot showing the expression [log 10(RPHM+1)] of paMAP-coding sequences in the iPSCs from this study and the PSCs from (Churko et al., 2017), with iPSCs grouped according to the method used for reprogramming. Data are represented as the median and inter-quartile range. p-values from pairwise Wilcoxon rank-sum test, adjusted for multiple comparisons using the Benjamini-Hochberg method. FIGs. G-H: Pearson correlations between observed retention times and predicted retention time (FIG. 2G) or hydrophobicity index (FIG. 2H).

FIG. 3 shows that paMAPs are shared across cancer types. Left panel: Heatmap showing the mean RNA expression [log 10(RPHM+1)] of paMAPs in cancer samples from our lab or TCGA, and the respective number of samples per cancer type in parentheses. Boxed: tissues with expression>2 RPHM in >10% of samples. Right panel: Bar plot showing the cumulative number of TCGA cancer types expressing the paMAP-coding sequence at different levels of sharing among samples. TCGA acronyms were used as defined by TCGA (portal.gdc.cancer.gov/). paMAPs in bold were previously reported.

FIGS. 4A-F show that high-stemness cancers acquire paMAP expression. FIG. 4A: Box plot showing the number of paMAPs expressed per TCGA sample within cancer types, in the increasing order of the median. FIG. 4B: Scatter plot displaying the number of paMAPs expressed across TCGA cancers (n=21 cancer types) according to the estimated tumor purity from (Aran et al., 2015). FIG. 4C: Scatter plot displaying the number of paMAPs versus the number of saMAPs expressed across TCGA cancers (n=32 cancer types, excluding TGCT). FIG. 4D: Mutation load [log 10(Non-synonymous mutations per mega base pairs+1)] in TCGA samples with no paMAP/saMAP expression (differentiated), with saMAP but no paMAP expression (stem-like), or with paMAP expression (pluripotent-like). Only samples with estimated purity>0.75 (Aran et al., 2015) were included (FIG. 10D). FIG. 4E: Volcano plot showing genes differentially expressed (red dots) between samples with high paMAP expression (>4 paMAPs, n=775) and high saMAP expression (>4 saMAPs and 0 paMAPs, n=1270). FIG. 4F: Boxplots showing the number of paMAPs at different molecular subtypes, grades, or stages of breast (BRCA), glioma (LGG.GBM), endometrial (UCEC) cancers, respectively, and within primary and metastatic melanoma (SKCM) samples. Each gray dot represents one sample.

FIGS. 5A-E show that shared epigenetic and signaling events associate with paMAP and saMAP expression across cancers. FIG. 5A: Heatmap showing the Spearman correlation between the paMAP expression (RPHM) and the methylation β-value at the promoter region of the respective source gene across cancers. All available data for the 450K methylation dataset were included. Boxed: p-adj<10⁻⁴ (Benjamini-Hochberg). FIG. 5B: Heatmap showing the Spearman correlation between the paMAP expression (RPHM) and the focal DNA copy number. Source gene symbols are added for reference; NA, no annotated source gene; all available data were included. Boxed: p-adj<10-4 (Benjamini-Hochberg). FIG. 5C: Within-cancer Spearman correlation between the number of paMAPs and saMAPs expressed per sample and the ssGSEA score for hallmark gene sets from MSigDB; only significant correlations are presented (p-adj<0.05, Benjamini-Hochberg), otherwise the cell is white. FIG. 5D: Prevalence of the indicated genomic feature in cancer samples that express paMAPs and saMAPs (>2 RPHM) versus those with no expression. The top three blocks were selected based on the highest prevalence in paMAP and saMAP-positive samples or lowest p-values. In contrast, features in the last block are PI3K/AKT signaling antagonists. p-value calculated based on the difference in prevalence in the two groups of samples using the Chi-square test. Features MUT, somatic mutation; Gain, single-copy gain; Amp, amplification; HL, heterozygous loss; HD, homozygous deletion. FIG. 5E: Heatmap showing the Spearman correlation between the number of paMAPs and saMAPs expressed and the expression of PRC2 components within cancer types. Boxed: correlations with p-adj<0.05 (Benjamini-Hochberg).

FIGS. 6A-C show the immunogenicity of paMAPs and saMAPs. FIG. 6A: Flow cytometry plots of peptide-HLA tetramer staining of specific CD8⁺ T-cells following in vitro stimulation, with numbers indicating the frequency of total CD8⁺ T cells. FIG. 6B: FEST assay showing the expansion of specific T cell clonotypes following in vitro stimulation with the indicated peptides alone or in a pool compared to the control without peptides (Tables 3A-B). FIG. 6C: Number of specific cells per million of CD8⁺ T cells in the pre-immune repertoire for each donor (D11-14), quantified using tetramer staining. N.D., not detected.

FIGS. 7A-D show that paMAP and saMAP expression correlates with immune evasion. FIG. 7A: Hazard ratio (risk of death) (±95% CI) for the association between the risk of death and the number of paMAPs with predicted presentation (#HLA-paMAPs), taking the number of paMAPs expressed (>0 RPHM) as a covariate. Red dots and whiskers, p-value<0.05 (Cox proportional-hazards model). Patients with more than one sample were excluded from the analysis. FIGS. 7B-D: Spearman correlation between the number of paMAPs and saMAPs expressed and the expression of MHC-I related genes (FIG. 7B), immune recruitment chemokine-encoding genes (FIG. 7C), or CDK4/6 genes (FIG. 7D) within cancer types. Boxed: correlations with p-adj<0.05 (Benjamini-Hochberg).

FIGS. 8A-C show an analysis of pluripotency markers and MHC expression after IFN-γ treatment. FIG. 8A, top panel: Representative flow cytometry profile of surface HLA-A, HLA-B and HLA-C(HLA-A,B,C) molecules on untreated and IFN-γ-treated Fibro-iPSC.2. FIG. 8A, bottom panel: Bar plot showing mean and standard deviation of the number of HLA-A,B,C molecules quantified using the QIFIKIT (see Example 1) for the three IFN-γ-treated and untreated iPSC samples. FIG. 8B: Representative flow cytometry profiles of pluripotency markers Oct4, SSEA-3, SSEA-4, and of differentiation marker SSEA-1 for the untreated and IFN-γ-treated Fibro-iPSC.2. FIG. 8C: Heatmap showing the clustering of the iPSCs in this study with PSCs from (Churko et al., 2017) and differentiated cells from different sources, using the ES1 set of genes from (Ben-Porath et al., 2008). BM, bone marrow; DC, dendritic cells; Fib, fibroblasts; ep, epithelial cells; ncpm, normalized counts per million; iso, isotype.

FIGS. 9A-F show that paMAPs and their source genes are highly expressed in PSCs but not in differentiated cells. FIG. 9A: Heatmap showing the RNA expression [log 10(RPHM+1)] of paMAPs across a panel of PSCs from (Churko et al., 2017) and the iPSCs from this study. Color code for each iPSC reprogramming method is shown. FIG. 9B: Bar plot shows the number of unique paMAPs identified per treatment, per cell line, and the number of paMAPs shared between the two conditions per cell line. FIG. 9C: Pearson correlation between the ssGSEA score for paMAP-source gene set and other published pluripotency-associated gene sets or the saMAP-source gene set from this study. FIG. 9D: Pearson correlation between the ssGSEA score for saMAP-source gene set and other published pluripotency-associated gene sets or the paMAP-source gene set from this study. FIG. 9E: ssGSEA score of paMAP- and saMAP-derived gene sets and other published pluripotency-associated gene sets in PSCs and differentiated cells from various sources (min-max-normalized across genesets). FIG. 9F: Overlap between the genes included in the respective gene sets.

FIGS. 10A-G show that paMAPs are expressed in high-stemness samples. FIG. 10A: Scatter plot displaying the number of saMAPs expressed across TCGA cancers (n=21 cancer types) according to the estimated tumor purity from (Aran et al., 2015). FIG. 10B, Left panel: Heatmap showing the mean RNA expression [log₁₀(RPHM+1)] of saMAPs in cancer samples from TCGA and the respective number of samples per cancer type in parentheses. Boxed: tissues with expression>2 RPHM in >10% of samples. FIG. 10B, Right panel: Bar plot showing the cumulative number of cancer types expressing the saMAP-coding sequence at different levels of sharing among within cancer types.

FIGS. 11A-F show the common epigenetic and signaling events associate with paMAP and saMAP expression across cancers. FIG. 11A: Heatmap showing the Spearman correlation between the paMAP expression (RPHM) and the methylation β-value at the promoter region of the respective source gene within cancers. All methylation data were obtained from the 450K methylation dataset, except for OV which contains data derived from the 27K methylation dataset. Boxed: p-adj<0.05 (Benjamini-Hochberg). FIG. 11B: Heatmap showing the Spearman correlation between the paMAP expression (RPHM) and the focal DNA copy number within cancers. Source gene symbols are added for reference; NA, no annotated source gene; all available data were included. FIG. 11C: Heatmap showing the Spearman correlation between the saMAP expression (RPHM) and the methylation β-value at the promoter region of the respective source gene across cancers. All available data for 450K methylation dataset were included. Boxed: p-adj<10⁻⁴ (Benjamini-Hochberg). FIG. 11D: Heatmap showing the Spearman correlation between the saMAP expression (RPHM) and the focal DNA copy number. Source gene symbols are added for reference; NA, no annotated source gene; all available data were included. Boxed: p-adj<10⁻⁴ (Benjamini-Hochberg). FIG. 11E: Within-cancer Spearman correlation between the number of paMAPs and saMAPs expressed per sample and the ssGSEA score for hallmark gene sets from the MSigDB, with purity estimates as a covariate; only significant correlations are presented (p-adj<0.05), otherwise the cell is white; only samples that had estimated purity from (Aran et al., 2015) were included. FIG. 11F: Heatmap shows the genes with the top three most prevalent mutations in cancer samples expressing paMAPs and saMAPs above the median number per cancer type. p-value>0.05, Fisher's exact test. Patients with more than one sample were excluded from the analysis.

FIGS. 12A-C show the expression of immunogenic paMAP- and saMAP-coding sequences in cancer and normal samples. FIG. 12A: Pie chart showing summary details of immunogenic paMAPs and saMAPs. Starting from the center: MAP type, biotype, class of ERE overlapping at genomic region (if applicable), source gene, MAP sequence. FIGS. 12B-C: MCS expression [log₁₀(RPHM+1)] of immunogenic paMAPs (FIG. 12B) and saMAPs (FIG. 12C), as determined in this or other studies, in the corresponding cancer types in which at least 10% of samples expressed the respective MAP (FIG. 3) and in the corresponding normal tissue from GTEx. *p<0.05, **p<0.01, ***p<0.001, ****p<0.0001 (Wilcoxon test).

FIGS. 13A-D show that paMAP and saMAP expression correlates with increased immune evasion. FIGS. 13A-B: Hazard ratio (±95% CI) for the association between the risk of death and the number of paMAPs expressed (>0 RPHM) (FIG. 13A), the number of saMAPs expressed (>0 RPHM) (FIG. 13B, left) or the number of saMAPs with predicted presentation (#HLA-saMAPs) taking the number of saMAPs expressed (>0 RPHM) as a covariate (FIG. 13B, right). Dots and whiskers, p-value<0.05 (Cox proportional-hazards model). Patients with more than one sample were excluded from the analysis. FIG. 13C: Spearman correlation between the immune cell infiltration score from xCell and the ssGSEA score for paMAP- and saMAP-source genes (left) or the number of paMAPs and saMAPs expressed above 2 RPHM (right), within cancer types. Boxed: correlations with p-adj<0.05 (Benjamini-Hochberg). FIG. 13D: Spearman correlation between the expression of immune inhibitory genes [from (Miranda et al., 2019; Thorsson et al., 2018)] and the ssGSEA score for paMAP- and saMAP-source genes (left) or the number of paMAPs and saMAPs expressed above 2 RPHM (right), within cancer types. Boxed: correlations with p-adj<0.05 (Benjamini-Hochberg).

### DETAILED DISCLOSURE

The use of the terms “a” and “an” and “the” and similar referents in the context of describing the technology (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

All methods described herein can be performed in any suitable order unless otherwise indicated herein or otherwise clearly contradicted by context.

The use of any and all examples, or exemplary language (“e.g.”, “such as”) provided herein, is intended merely to better illustrate embodiments of the claimed technology and does not pose a limitation on the scope unless otherwise claimed.

No language in the specification should be construed as indicating any non-claimed element as essential to the practice of embodiments of the claimed technology.

Herein, the term “about” has its ordinary meaning. The term “about” is used to indicate that a value includes an inherent variation of error for the device or the method being employed to determine the value, or encompass values close to the recited values, for example within 10% of the recited values (or range of values).

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

Where features or aspects of the disclosure are described in terms of Markush groups or list of alternatives, those skilled in the art will recognize that the disclosure is also thereby described in terms of any individual member, or subgroup of members, of the Markush group or list of alternatives.

Unless specifically defined otherwise, all technical and scientific terms used herein shall be taken to have the same meaning as commonly understood by one of ordinary skill in the art (e.g., in stem cell biology, cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

In the studies described herein, the present inventors have identified tumor-specific antigen (TSA) and tumor-associated antigen (TAA) candidates from human iPSCs using a proteogenomic-based approach. Several pluripotency-associated MHC-I-associated peptides (paMAPs) that are absent from the transcriptome of normal tissues and adult stem cells but expressed in pluripotent stem cells (PSCs) and multiple cancer types were identified. These paMAPs derived from coding and allegedly non-coding (48%) transcripts involved in pluripotency maintenance, and their expression in samples correlated with source gene hypomethylation and genomic aberrations common across cancer types. The novel TSA and TAA candidates identified herein may be useful, e.g., for T-cell based immunotherapy and vaccines against cancer stem cells (CSCs), for example for the treatment of poorly differentiated cancers.

Accordingly, in an aspect, the present disclosure relates to a cancer stem cell (CSC) tumor antigen peptide (TAP) (or CSC tumor-specific peptide) comprising, or consisting of, one of the following amino acid sequences:

| Peptide | SEQ ID | Peptide | SEQ ID |
|---|---|---|---|
| sequence | NO: | sequence | NO: |
| NTENYILWGY | 1 | ISMCDLVY | 21 |
| MKFGNQVSGLF | 2 | YKRMKLDSY | 22 |
| RLQHEPPHPV | 3 | QPLPEPLQLW | 23 |
| LPMWKALLF | 4 | IPMKIYLW) | 24 |
| RPARPPAGL | 5 | SQSSLMLYL | 25 |
| FAYPNQKVTF | 6 | ALYPQPPTV | 26 |
| GQAHPQGSF | 7 | YTPFPSYGHY | 27 |
| YSDQKPPYSY | 8 | FTEEDLHFVLY | 28 |
| KLAQIIRQV | 9 | GQITHNTSF | 29 |
| GEIKTFSDL | 10 | VTLSTYFHV | 30 |
| GKLDNTNEY | 11 | GQFDRPAGV | 31 |
| AKKKENITY | 12 | VSDQQNGTY | 32 |
| SAQGKPTYF | 13 | KHFDSPRGVAF | 33 |
| EEEIHSPTL | 14 | GEHLVSVTL | 34 |
| SKLRSTGQSF | 15 | YSIYPMRNL | 35 |
| NTLSESYIY | 16 | SQNSPIRY | 36 |
| QPLPQPLELW | 17 | FHSQNSPIRY | 37 |
| RTDTGKRVLY | 18 | VAKPPGTAF | 38 |
| LPSGETIAKW | 19 | SLLGSSEILEV | 39 |
| KLDSYIIPY | 20 | | |
| Peptide | SEQ ID | Peptide | SEQ ID |
| sequence | NO: | sequence | NO: |
| GKFQGLIEKF | 47 | ALLEGVNTVVV | 55 |
| KQMENDIQLY | 48 | SETHPPEVAL | 56 |
| ASTPASSEL | 49 | SPQEASGVRW | 57 |
| RLWNETVELF | 50 | YLLNCHLLI | 58 |
| FGDGKFSEV | 51 | GQFLVKSGY | 59 |
| SEVSADKLVAL | 52 | QTELNNSKQEY | 60 |
| AMYHALEKA | 53 | MAWNGILHL | 61 |
| FAYPNQKDF | 54 | HPLPGLILEW | 62 |

In general, peptides such as tumor antigen peptides (TAPs) presented in the context of HLA class I vary in length from about 7 or 8 to about 15, or preferably 8 to 14 amino acid residues. In some embodiments of the methods of the disclosure, longer peptides comprising the TAP sequences defined herein are artificially loaded into cells such as antigen presenting cells (APCs), processed by the cells and the TAP is presented by MHC class I molecules at the surface of the APC. In this method, peptides/polypeptides longer than 15 amino acid residues can be loaded into APCs, are processed by proteases in the APC cytosol providing the corresponding TAP as defined herein for presentation. In some embodiments, the precursor peptide/polypeptide that is used to generate the TAP defined herein is for example 1000, 500, 400, 300, 200, 150, 100, 75, 50, 45, 40, 35, 30, 25, 20 or 15 amino acids or less. Thus, all the methods and processes using the TAPs described herein include the use of longer peptides or polypeptides (including the native protein), i.e. tumor antigen precursor peptides/polypeptides, to induce the presentation of the “final” 8-14 TAP following processing by the cell (APCs). In some embodiments, the herein-mentioned TAP is about 8 to 14, 8 to 13, or 8 to 12 amino acids long (e.g., 8, 9, 10, 11, 12 or 13 amino acids long), small enough for a direct fit in an HLA class I molecule. In an embodiment, the TAP comprises 20 amino acids or less, preferably 15 amino acids or less, more preferably 14 amino acids or less. In an embodiment, the TAP comprises at least 7 amino acids, preferably at least 8 amino acids or less, more preferably at least 9 amino acids.

The term “amino acid” as used herein includes both L- and D-isomers of the naturally occurring amino acids as well as other amino acids (e.g., naturally-occurring amino acids, non-naturally-occurring amino acids, amino acids which are not encoded by nucleic acid sequences, etc.) used in peptide chemistry to prepare synthetic analogs of TAPs. Examples of naturally occurring amino acids are glycine, alanine, valine, leucine, isoleucine, serine, threonine, etc. Other amino acids include for example non-genetically encoded forms of amino acids, amino acid analogs as well as a conservative substitution of an L-amino acid. Naturally-occurring non-genetically encoded amino acids and amino acid analogs include, for example, beta-alanine, 3-amino-propionic acid, 2,3-diaminopropionic acid, alpha-aminoisobutyric acid (Aib), 4-amino-butyric acid, N-methylglycine (sarcosine), hydroxyproline, ornithine (e.g., L-ornithine), citrulline, t-butylalanine, t-butylglycine, N-methylisoleucine, phenylglycine, cyclohexylalanine, norleucine (Nle), norvaline, 2-napthylalanine, pyridylalanine, 3-benzothienyl alanine, 4-chlorophenylalanine, 2-fluorophenylalanine, 3-fluorophenylalanine, 4-fluorophenylalanine, penicillamine, 1,2,3,4-tetrahydro-isoquinoline-3-carboxylix acid, beta-2-thienylalanine, methionine sulfoxide, L-homoarginine (Hoarg), N-acetyl lysine, 2-amino butyric acid, 2-amino butyric acid, 2,4,-diaminobutyric acid (D- or L-), p-aminophenylalanine, N-methylvaline, homocysteine, homoserine (HoSer), cysteic acid, epsilon-amino hexanoic acid, delta-amino valeric acid, benzyloxy-tyrosine, β-phenylalanine or 2,3-diaminobutyric acid (D- or L-), etc. These amino acids are well known in the art of biochemistry/peptide chemistry. Thus, one or more of the amino acids in the CSC TAPs described herein (SEQ ID NO:1-62) may be replaced by a non-genetically encoded amino acid and/or an amino acid analog. The TAPs may also be modified to improve the proteolytic stability of the peptides, for example by the incorporation of methyl-amino acids, β-amino acids or peptoids. In an embodiment, the TAP comprises only naturally-occurring amino acids.

In embodiments, the TAPs described herein include peptides with altered sequences containing substitutions of functionally equivalent amino acid residues, relative to the herein-mentioned sequences. For example, one or more amino acid residues within the sequence can be substituted by another amino acid (or an amino acid analog) of a similar polarity (having similar physico-chemical properties) which acts as a functional equivalent, resulting in a silent alteration. Substitution for an amino acid within the sequence may be selected from other members of the class to which the amino acid belongs. For example, positively charged (basic) amino acids include arginine, lysine and histidine (as well as homoarginine and ornithine). Nonpolar (hydrophobic) amino acids include leucine, isoleucine, alanine, phenylalanine, valine, proline, tryptophan and methionine. Uncharged polar amino acids include serine, threonine, cysteine, tyrosine, asparagine and glutamine. Negatively charged (acidic) amino acids include glutamic acid and aspartic acid. The amino acid glycine may be included in either the nonpolar amino acid family or the uncharged (neutral) polar amino acid family. Substitutions made within a family of amino acids are generally understood to be conservative substitutions. The herein-mentioned TAP may comprise all L-amino acids, all D-amino acids or a mixture of L- and D-amino acids. In an embodiment, the herein-mentioned TAP comprises all L-amino acids.

In an embodiment, in the sequences of the TAPs comprising or consisting of one of sequences of SEQ ID NOs: 1-39 and 47-62, the amino acid residues that do not substantially contribute to interactions with the T-cell receptor may be modified by replacement with other amino acid whose incorporation does not substantially affect T-cell reactivity and does not eliminate binding to the relevant MHC.

The TAP may also be modified by replacing one or more of the amide bonds of the peptide that may improve chemical stability and/or enhanced biological/pharmacological properties (e.g., half-life, absorption, potency, efficiency, etc.). Typical peptide bond replacements include esters, polyamines and derivatives thereof as well as substituted alkanes and alkenes, such as aminomethyl and ketomethylene. For example, the above-mentioned TAP may have one or more amide bonds replaced by linkages such as —CH₂NH—, —CH₂S—, —CH₂—CH₂—, —CH═CH— (cis or trans), —CH₂SO—, —CH(OH)CH₂—, or —COCH₂—.

The TAP may also be N- and/or C-terminally capped or modified to prevent degradation, increase stability, affinity and/or uptake. Thus, in another aspect, the present disclosure provides a modified TAP of the formula Z¹—X—Z², wherein X is a TAP comprising, or consisting of, one of the amino acid sequences of SEQ ID NOs: 1-39 and 47-62.

In an embodiment, the amino terminal residue (i.e., the free amino group at the N-terminal end) of the TAP is modified (e.g., for protection against degradation), for example by covalent attachment of a moiety/chemical group (Z¹). Z¹ may be a straight chained or branched alkyl group of one to eight carbons, or an acyl group (R—CO—), wherein R is a hydrophobic moiety (e.g., acetyl, propionyl, butanyl, iso-propionyl, or iso-butanyl), or an aroyl group (Ar—CO—), wherein Ar is an aryl group. In an embodiment, the acyl group is a C₁-C₁₆ or C₃-C₁₆ acyl group (linear or branched, saturated or unsaturated), in a further embodiment, a saturated C₁-C₆ acyl group (linear or branched) or an unsaturated C₃-C₆ acyl group (linear or branched), for example an acetyl group (CH₃—CO—, Ac). In an embodiment, Z¹ is absent. The carboxy terminal residue (i.e., the free carboxy group at the C-terminal end of the TAP) of the TAP may be modified (e.g., for protection against degradation), for example by amidation (replacement of the OH group by a NH₂ group), thus in such a case Z² is a NH₂ group. In an embodiment, Z² may be an hydroxamate group, a nitrile group, an amide (primary, secondary or tertiary) group, an aliphatic amine of one to ten carbons such as methyl amine, iso-butylamine, iso-valerylamine or cyclohexylamine, an aromatic or arylalkyl amine such as aniline, napthylamine, benzylamine, cinnamylamine, or phenylethylamine, an alcohol or CH₂OH. In an embodiment, Z² is absent. In an embodiment, the TAP comprises one of the amino acid sequences of SEQ ID NOs: 1-39 and 47-62. In an embodiment, the TAP consists of one of the amino acid sequences of SEQ ID NOs: 1-39 and 47-62, i.e., wherein Z¹ and Z² are absent.

In an embodiment, the TAP of the present disclosure comprises or consists of one of the amino acid sequences of SEQ ID NOs: 1-39.

In another aspect, the present disclosure provides a CSC TAP (or tumor-specific peptide) binding to an HLA-A*01:01 molecule, comprising or consisting of the sequence of SEQ ID NO:1, 8, 16, 20, 21, 27, 28, 32, 37 or 60, preferably SEQ ID NO:1, 8, 16, 20, 21, 27, 28, 32 or 37.

In another aspect, the present disclosure provides a CSC TAP (or tumor-specific peptide) binding to an HLA-A*02:01 molecule, comprising or consisting of the sequence of SEQ ID NO:3, 6, 26, 30, 31, 39, 53, 55 or 58 preferably SEQ ID NO: 3, 6, 26, 30, 31 or 39. Because HLA alleles show promiscuity (certain HLA alleles present similar epitopes), the above-identified TAP may further bind to HLA-A*02:05, HLA-A*02:06 and/or HLA-A*02:07 molecules.

In another aspect, the present disclosure provides a CSC TAP (or tumor-specific peptide) binding to an HLA-B*07:02 molecule, comprising or consisting of the sequence of SEQ ID NO:5. Because HLA alleles show promiscuity (certain HLA alleles present similar epitopes), the above-identified TAP may further bind to HLA-B*35:02, HLA-B*35:03, HLA-B*55:01 and/or HLA-B*56:01 molecules.

In another aspect, the present disclosure provides a CSC TAP (or tumor-specific peptide) binding to an HLA-B*15:03 molecule, comprising or consisting of the sequence of SEQ ID NO:2, 7, 11, 12, 15, 22, 29, 36, 38, 47, 48, or 59, preferably SEQ ID NO:2, 7, 11, 12, 15, 22, 29, 36 or 38. Because HLA alleles show promiscuity (certain HLA alleles present similar epitopes), the above-identified TAP may further bind to HLA-B*15:01, HLA-B*15:02 and/or HLA-B*46:01 molecules.

In another aspect, the present disclosure provides a CSC TAP (or tumor-specific peptide) binding to an HLA-B*40:01 molecule, comprising or consisting of the sequence of SEQ ID NO:10, 25, 34, 52 or 56, preferably SEQ ID NO:10, 25 or 34. Because HLA alleles show promiscuity (certain HLA alleles present similar epitopes), the above-identified TAP may further bind to HLA-B*18:01, HLA-B*40:02, HLA-B*41:02, HLA-B*44:02, HLA-B*44:03 and/or HLA-B*45:01 molecules.

In another aspect, the present disclosure provides a CSC TAP (or tumor-specific peptide) binding to an HLA-B*53:01 molecule, comprising or consisting of the sequence of SEQ ID NO:4, 17, 19, 23, 24 or 57, preferably SEQ ID NO: 4, 17, 19, 23 or 24. Because HLA alleles show promiscuity (certain HLA alleles present similar epitopes), the above-identified TAP may further bind to HLA-B*35:02, HLA-B*35:03, HLA-B*52:01, HLA-B*51:01, HLA-B*55:01 and/or HLA-B*56:01 molecules.

In another aspect, the present disclosure provides a CSC TAP (or tumor-specific peptide) binding to an HLA-C*02:10 molecule, comprising or consisting of the sequence of SEQ ID NO:6, 54 or 61, preferably SEQ ID NO:6.

In another aspect, the present disclosure provides a CSC TAP (or tumor-specific peptide) binding to an HLA-C*03:04 molecule, comprising or consisting of the sequence of SEQ ID NO:6, 35, 49 or 51, preferably SEQ ID NO:6 or 35. Because HLA alleles show promiscuity (certain HLA alleles present similar epitopes), the above-identified TAP may further bind to HLA-B*46:01, HLA-C*03:02, HLA-C*08:01, HLA-C*08:02, HLA-C*12:02, HLA-C*12:03, HLA-C*15:02 and/or HLA-C*16:01 molecules.

In another aspect, the present disclosure provides a CSC TAP (or tumor-specific peptide) binding to an HLA-C*04:01 molecule, comprising or consisting of the sequence of SEQ ID NO: 13, 33, 50, preferably SEQ ID NO: 13 or 33. Because HLA alleles show promiscuity (certain HLA alleles present similar epitopes), the above-identified TAP may further bind to HLA-C*07:02 and/or HLA-C*14:02 molecules.

The TAPs of the disclosure may be produced by expression in a host cell comprising a nucleic acid encoding the TAPs (recombinant expression) or by chemical synthesis (e.g., solid-phase peptide synthesis). Peptides can be readily synthesized by manual and/or automated solid phase procedures well known in the art. Suitable syntheses can be performed for example by utilizing “T-boc” or “Fmoc” procedures. Techniques and procedures for solid phase synthesis are described in for example Solid Phase Peptide Synthesis: A Practical Approach, by E. Atherton and R. C. Sheppard, published by IRL, Oxford University Press, 1989. Alternatively, the MiHA peptides may be prepared by way of segment condensation, as described, for example, in Liu et al., Tetrahedron Lett. 37: 933-936, 1996; Baca et al., J. Am. Chem. Soc. 117: 1881-1887, 1995; Tam et al., Int. J. Peptide Protein Res. 45: 209-216, 1995; Schnolzer and Kent, Science 256: 221-225, 1992; Liu and Tam, J. Am. Chem. Soc. 116: 4149-4153, 1994; Liu and Tam, Proc. Natl. Acad. Sci. USA 91: 6584-6588, 1994; and Yamashiro and Li, Int. J. Peptide Protein Res. 31: 322-334, 1988). Other methods useful for synthesizing the TAPs are described in Nakagawa et al., J. Am. Chem. Soc. 107: 7087-7092, 1985. In an embodiment, the TAP is chemically synthesized (synthetic peptide). Another embodiment of the present disclosure relates to a non-naturally occurring peptide wherein said peptide consists or consists essentially of an amino acid sequences defined herein and has been synthetically produced (e.g., synthesized) as a pharmaceutically acceptable salt. The salts of the TAPs according to the present disclosure differ substantially from the peptides in their state(s) in vivo, as the peptides as generated in vivo are no salts. The non-natural salt form of the peptide may modulate the solubility of the peptide, in particular in the context of pharmaceutical compositions comprising the peptides, e.g. the peptide vaccines as disclosed herein. Preferably, the salts are pharmaceutically acceptable salts of the peptides.

In an embodiment, the herein-mentioned TAP is substantially pure. A compound is “substantially pure” when it is separated from the components that naturally accompany it. Typically, a compound is substantially pure when it is at least 60%, more generally 75%, 80% or 85%, preferably over 90% and more preferably over 95%, by weight, of the total material in a sample. Thus, for example, a polypeptide that is chemically synthesized or produced by recombinant technology will generally be substantially free from its naturally associated components, e.g. components of its source macromolecule. A nucleic acid molecule is substantially pure when it is not immediately contiguous with (i.e., covalently linked to) the coding sequences with which it is normally contiguous in the naturally occurring genome of the organism from which the nucleic acid is derived. A substantially pure compound can be obtained, for example, by extraction from a natural source; by expression of a recombinant nucleic acid molecule encoding a peptide compound; or by chemical synthesis. Purity can be measured using any appropriate method such as column chromatography, gel electrophoresis, HPLC, etc. In an embodiment, the TAP is in solution. In another embodiment, the TAP is in solid form, e.g., lyophilized.

In an embodiment, the TAP is encoded by a sequence located a non-protein coding region of the genome. In an embodiment, the TAP is encoded by a sequence located in an untranslated transcribed region (UTR), i.e., a 3′-UTR or 5′-UTR region. In another embodiment, the TAP is encoded by a sequence located in an intron. In another embodiment, the TAP is encoded by a sequence located in an intergenic region. In another embodiment, the TAP is encoded by a sequence located in an exon and originates from a frameshift.

In another aspect, the disclosure further provides a nucleic acid (isolated) encoding the herein-mentioned TAPs or a tumor antigen precursor-peptide. In an embodiment, the nucleic acid comprises from about 21 nucleotides to about 45 nucleotides, from about 24 to about 45 nucleotides, for example 24, 27, 30, 33, 36, 39, 42 or 45 nucleotides. “Isolated”, as used herein, refers to a peptide or nucleic acid molecule separated from other components that are present in the natural environment of the molecule or a naturally occurring source macromolecule (e.g., including other nucleic acids, proteins, lipids, sugars, etc.). Synthetic, as used herein, refers to a peptide or nucleic molecule that is not isolated from its natural sources, e.g., which is produced through recombinant technology or using chemical synthesis. In an embodiment, the nucleic acid (DNA, RNA) encoding the TAP of the disclosure comprises any one of the sequences set forth in the tables below or a corresponding RNA sequence. In an embodiment, the nucleic acid encoding the TAP is an mRNA molecule.

| Peptide | Encoding | Peptide | Encoding |
|---|---|---|---|
| sequence | nucleotide sequence | sequence | nucleotide sequence |
| NTENYILWGY | AACACAGAAAATTATATACTCT | ISMCDLVY | ATTTCTATGTGTGATTTGGT |
| | GGGGTTAC (SEQ ID NO: 88) | | TTAT (SEQ ID NO: 108) |
| MKFGNQVSGLF | ATGAAATTTGGTAATCAGGTAA | YKRMKLDSY | TACAAAAGAATGAAGTTGGA |
| | GCGGACTCTTC (SEQ ID | | CTCTTAC (SEQ ID NO: 109) |
| | NO: 89) | | |
| RLQHEPPHPV | AGATTACAGCATGAGCCACCA | QPLPEPLQLW | CAGCCACTCCCAGAGCCCC |
| | CACCCTGTC (SEQ ID NO: 90) | | TGCAACTCTGG (SEQ ID |
| | | | NO: 110) |
| LPMWKALLF | CTGCCCATGTGGAAGGCTCTG | IPMKIYLW) | ATCCCCATGAAAATATATCT |
| | CTGTTT (SEQ ID NO: 91) | | CTGG (SEQ ID NO: 111) |
| RPARPPAGL | CGCCCTGCACGGCCGCCAGC | SQSSLMLYL | TCCCAGAGCTCACTAATGCT |
| | CGGCCTC (SEQ ID NO: 92) | | ATATTTA (SEQ ID NO: 112) |
| FAYPNQKVTF | TTTGCCTACCCAAATCAAAAG | ALYPQPPTV | GCGCTGTATCCTCAGCCGC |
| | GTAACTTTT (SEQ ID NO: 93) | | CCACTGTG (SEQ ID NO: 113) |
| GQAHPQGSF | GGTCAAGCTCACCCCCAAGG | YTPFPSYGHY | TACACGCCTTTCCCGTCCTA |
| | CAGCTTC (SEQ ID NO: 94) | | TGGACACTAC (SEQ ID |
| | | | NO: 114) |
| YSDQKPPYSY | TACAGCGACCAGAAGCCGCC | FTEEDLHFVLY | TTCACGGAGGAGGACCTGC |
| | CTACTCGTAC (SEQ ID NO: 95) | | ACTTCGTTCTGTAC (SEQ ID |
| | | | NO: 115) |
| KLAQIIRQV | AAGCTGGCCCAGATCATCCGT | GQITHNTSF | GGGCAAATCACACATAACA |
| | CAGGTC (SEQ ID NO: 96) | | CTTCATTC (SEQ ID NO: 116) |
| GEIKTFSDL | GGTGAAATAAAGACATTTTCA | VTLSTYFHV | GTCACCTTGAGTACTTATTT |
| | GATCTG (SEQ ID NO: 97) | | CCATGTG (SEQ ID NO: 117) |
| GKLDNTNEY | GGTAAATTAGACAACACGAAC | GQFDRPAGV | GGGCAGTTCGACCGACCAG |
| | GAATAC (SEQ ID NO: 98) | | CCGGCGTG (SEQ ID |
| | | | NO: 118) |
| AKKKENITY | GCAAAGAAAAAGGAAAATATC | VSDQQNGTY | GTGAGTGATCAGCAGAATG |
| | ACCTAT (SEQ ID NO: 99) | | GGACATAC (SEQ ID NO: 119) |
| SAQGKPTYF | AGTGCACAGGGAAAGCCAAC | KHFDSPRGVAF | AAGCACTTTGACTCCCCAC |
| | CTACTTT (SEQ ID NO: 100) | | GGGGTGTGGCCTTC (SEQ |
| | | | ID NO: 120) |
| EEEIHSPTL | GAAGAAGAAATCCACAGCCCT | GEHLVSVTL | GGTGAGCACCTGGTATCTG |
| | ACCCTG (SEQ ID NO: 101) | | TGACACTG (SEQ ID NO: 121) |
| SKLRSTGQSF | TCCAAACTGAGAAGCACTGGA | YSIYPMRNL | TACTCCATCTACCCCATGCG |
| | CAGTCATTT (SEQ ID NO: 102) | | CAACCTC (SEQ ID NO: 122) |
| NTLSESYIY | AACACCCTGAGTGAATCTTAC | SQNSPIRY | TCTCAAAACTCTCCTATAAG |
| | ATTTAT (SEQ ID NO: 103) | | GTAT (SEQ ID NO: 123) |
| QPLPQPLELW | CAGCCACTTCCACAGCCCCTG | FHSQNSPIRY | TTTCATTCTCAAAACTCTCC |
| | GAACTCTGG (SEQ ID NO: 104) | | TATAAGGTAT (SEQ ID |
| | | | NO: 124) |
| RTDTGKRVLY | AGGACTGACACAGGTAAGAGA | VAKPPGTAF | GTAGCCAAACCCCCAGGAA |
| | GTCTTGTAT (SEQ ID NO: 105) | | CTGCTTTC (SEQ ID NO: 125) |
| LPSGETIAKW | TTGCCAAGCGGTGAGACTATC | SLLGSSEILEV | TCCCTGCTTGGCAGCAGTG |
| | GCCAAGTGG (SEQ ID NO: 106) | | AAATTTTGGAGGTC (SEQ ID |
| | | | NO: 126) |
| KLDSYIIPY | AAGTTGGACTCTTACATTATAC | | |
| | CATAT (SEQ ID NO: 107) | | |
| Peptide | Encoding | Peptide | Encoding |
| sequence | nucleotide sequence | sequence | nucleotide sequence |
| GKFQGLIEKF | GGGAAGTTCCAGGGTCTGATT | ALLEGVNTVVV | GCGCTCCTTGAGGGAGTTA |
| | GAGAAGTTT (SEQ ID NO: 127) | | ATACAGTTGTGGTG (SEQ ID |
| | | | NO: 135) |
| KQMENDIQLY | AAGCAAATGGAAAATGATATTC | SETHPPEVAL | TCTGAGACACATCCTCCTGA |
| | AGTTATAT (SEQ ID NO: 128) | | AGTGGCTCTT (SEQ ID |
| | | | NO: 136) |
| ASTPASSEL | GCCTCCACTCCAGCCTCATCAG | SPQEASGVRW | TCTCCACAAGAGGCCTCTG |
| | AGTTA (SEQ ID NO: 129) | | GTGTCAGGTGG (SEQ ID |
| | | | NO: 137) |
| RLWNETVELF | AGGCTGTGGAATGAGACCGTG | YLLNCHLLI | TACCTTCTAAACTGCCACCT |
| | GAGCTTTTT (SEQ ID NO: 130) | | GTTAATC (SEQ ID NO: 138) |
| FGDGKFSEV | TTTGGCGATGGCAAGTTCTCCG | GQFLVKSGY | GGCCAGTTCTTGGTCAAAT |
| | AGGTC (SEQ ID NO: 131) | | CCGGCTAC (SEQ ID NO: 139) |
| SEVSADKLVAL | TCCGAGGTCTCTGCAGACAAAC | QTELNNSKQEY | CAGACAGAGCTGAATAATTC |
| | TGGTGGCACTG (SEQ ID | | AAAGCAAGAATAT (SEQ ID |
| | NO: 132) | | NO: 140) |
| AMYHALEKA | GCCATGTACCATGCTCTGGAGA | MAWNGILHL | ATGGCATGGAATGGAATCC |
| | AAGCC (SEQ ID NO: 133) | | TACACCTC (SEQ ID NO: 141) |
| FAYPNQKDF | TTTGCCTACCCAAATCAAAAGG | HPLPGLILEW | CATCCTTTACCAGGCTTGAT |
| | ATTTT (SEQ ID NO: 134) | | ATTAGAATGG (SEQ ID |
| | | | NO: 142) |

A nucleic acid of the disclosure may be used for recombinant expression of the TAP of the disclosure, and may be included in a vector or plasmid, such as a cloning vector or an expression vector, which may be transfected into a host cell. In an embodiment, the disclosure provides a cloning, expression or viral vector or plasmid comprising a nucleic acid sequence encoding the TAP of the disclosure. Alternatively, a nucleic acid encoding a TAP of the disclosure may be incorporated into the genome of the host cell. In either case, the host cell expresses the TAP or protein encoded by the nucleic acid. The term “host cell” as used herein refers not only to the particular subject cell, but to the progeny or potential progeny of such a cell. A host cell can be any prokaryotic (e.g., E. coli) or eukaryotic cell (e.g., insect cells, yeast or mammalian cells) capable of expressing the TAPs described herein. The vector or plasmid contains the necessary elements for the transcription and translation of the inserted coding sequence, and may contain other components such as resistance genes, cloning sites, etc. Methods that are well known to those skilled in the art may be used to construct expression vectors containing sequences encoding peptides or polypeptides and appropriate transcriptional and translational control/regulatory elements operably linked thereto. These methods include in vitro recombinant DNA techniques, synthetic techniques, and in vivo genetic recombination. Such techniques are described in Sambrook. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York, N.Y. “Operably linked” refers to a juxtaposition of components, particularly nucleotide sequences, such that the normal function of the components can be performed. Thus, a coding sequence that is operably linked to regulatory sequences refers to a configuration of nucleotide sequences wherein the coding sequences can be expressed under the regulatory control, that is, transcriptional and/or translational control, of the regulatory sequences. “Regulatory/control region” or “regulatory/control sequence”, as used herein, refers to the non-coding nucleotide sequences that are involved in the regulation of the expression of a coding nucleic acid. Thus, the term regulatory region includes promoter sequences, regulatory protein binding sites, upstream activator sequences, and the like. The vector (e.g., expression vector) may have the necessary 5′ upstream and 3′ downstream regulatory elements such as promoter sequences such as CMV, PGK and EFIa promoters, ribosome recognition and binding TATA box, and 3′ UTR AAUAAA transcription termination sequence for the efficient gene transcription and translation in its respective host cell. Other suitable promoters include the constitutive promoter of simian vims 40 (SV40) early promoter, mouse mammary tumor virus (MMTV), HIV LTR promoter, MoMuLV promoter, avian leukemia virus promoter, EBV immediate early promoter, and Rous sarcoma vims promoter. Human gene promoters may also be used, including, but not limited to the actin promoter, the myosin promoter, the hemoglobin promoter, and the creatine kinase promoter. In certain embodiments inducible promoters are also contemplated as part of the vectors expressing the TAP. This provides a molecular switch capable of turning on expression of the polynucleotide sequence of interest or turning off expression. Examples of inducible promoters include, but are not limited to a metallothionine promoter, a glucocorticoid promoter, a progesterone promoter, or a tetracycline promoter. Examples of vectors are plasmid, autonomously replicating sequences, and transposable elements. Additional exemplary vectors include, without limitation, plasmids, phagemids, cosmids, artificial chromosomes such as yeast artificial chromosome (YAC), bacterial artificial chromosome (BAC), or PI-derived artificial chromosome (PAC), bacteriophages such as lambda phage or M13 phage, and animal viruses. Examples of categories of animal viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40). Examples of expression vectors are Lenti-X™ Bicistronic Expression System (Neo) vectors (Clontrch), pClneo vectors (Promega) for expression in mammalian cells; pLenti4/V5-DEST™ pLenti6/V5-DEST™, and pLenti6.2N5-GW/lacZ (Invitrogen) for lentivirus-mediated gene transfer and expression in mammalian cells. The coding sequences of the TAPs disclosed herein can be ligated into such expression vectors for the expression of the TAP in mammalian cells.

In certain embodiments, the nucleic acids encoding the TAP of the present disclosure are provided in a viral vector. A viral vector can be those derived from retrovirus, lentivirus, or foamy virus. As used herein, the term, “viral vector,” refers to a nucleic acid vector construct that includes at least one element of viral origin and has the capacity to be packaged into a viral vector particle. The viral vector can contain the coding sequence for the various proteins described herein in place of nonessential viral genes. The vector and/or particle can be utilized for the purpose of transferring DNA, RNA or other nucleic acids into cells either in vitro or in vivo. Numerous forms of viral vectors are known in the art.

In embodiment, the nucleic acid (DNA, RNA) encoding the TAP of the disclosure is comprised within a vesicle or nanoparticle such as a lipid vesicle (e.g., liposome) or lipid nanoparticle (LNP), or any other suitable vehicle. Thus, in another aspect, the present disclosure provides a lipid vesicle or nanoparticle comprising a nucleic acid, such as an mRNA, encoding one or more of the CSC TAP described herein.

The term liposome as used herein in accordance with its usual meaning, referring to microscopic lipid vesicles composed of a bilayer of phospholipids or any similar amphipathic lipids (e.g., sphingolipids) encapsulating an internal aqueous medium.

The term “lipid nanoparticle” refers to liposome-like structure that may include one or more lipid bilayer rings surrounding an internal aqueous medium similar to liposomes, or micellar-like structures that encapsulates molecules (e.g., nucleic acids) in a non-aqueous core. Lipid nanoparticles typically contain cationic lipids, such as ionizable cationic lipids. Examples of cationic lipids that may be used for LNPs include DOTMA, DOSPA, DOTAP, ePC, DLin-MC3-DMA, C12-200, ALC-0315, cKK-E12, Lipid H (SM-102), OF-Deg-Lin, A2-Iso5-2DC18, 306Oᵢ₁₀, BAME-O16B, TT3, 9A1P9, FTT5, COATSOME® SS-E, COATSOME® SS-EC, COATSOME® SS-OC and COATSOME® SS-OP (see, e.g., Hou et al., Nature Reviews Materials, volume 6, pages 1078-1094 (2021); Tenchov et al., ACS Nano, 15, 16982-17015 (2021).

Liposomes and lipid nanoparticles typically include other lipid components such as lipids, lipid-like materials, and polymers that can improve liposome or nanoparticle properties, such as stability, delivery efficacy, tolerability and biodistribution. These include phospholipids (e.g., phosphatidylcholines, phosphatidylethanolamines, phosphatidylserines, and phosphatidylglycerol) such as 1,2-distearoyl-sn-glycero-3-phosphocholine (DSPC) and DOPE, sterols (such as cholesterol and cholesterol derivatives), PEGylated lipids (PEG-lipids) such as 1,2-dimyristoyl-rac-glycero-3-methoxypolyethylene glycol-2000 (PEG₂₀₀₀-DMG) and 1,2-distearoyl-rac-glycero-3-methoxypolyethylene glycol-2000 (PEG₂₀₀₀-DSG).

In an embodiment, the lipid nanoparticle according to the present disclosure comprises one or more cationic lipids, such as ionizable cationic lipids.

The nucleic acid (e.g., mRNA) encoding one or more of the CSC TAP, may be modified, for example to increase stability and/or reduce immunogenicity. For example, the 5′ end may be capped to stabilize the molecule and decrease immunogenicity (for example, as described in U.S. Ser. No. 10/519,189 and U.S. Ser. No. 10/494,399). One or more nucleosides of the mRNA may be modified or substituted with 1-methyl pseudo-uridine to either increase stability of the molecule or reduce recognition of the molecule by the innate immune system. A form of modified nucleosides are described in U.S. Pat. No. 9,371,511. Other types of modifications that may be made to the mRNA include incorporation of anti-reverse cap analog (ARCA), 5′-methyl-cytidine triphosphate (m5CTP), N6-methyl-adenosine-5′-triphosphate (m6ATP), 2-thio-uridine triphosphate (s2UTP), pseudouridine triphosphate, N¹Methylpseudouridine triphosphate or 5-Methoxyuridine triphosphate (5moUTP). The mRNA may also include additional modifications to the 5′- and/or 3′-untranslated regions (UTRs) and polyadenylation (polyA) tail (see, for example, Kim et al., Molecular & cellular toxicology vol. 18, 1 (2022): 1-8). All these modifications and other modifications to the nucleic acid (e.g., mRNA) encoding the CSC TAP are encompassed by the present disclosure.

In another aspect, the present disclosure provides an MHC class I molecule comprising (i.e., presenting or bound to) one or more of the TAP of SEQ ID NOs: 1-39 and 47-62.

In an embodiment, the MHC class I molecule is an HLA-A*01:01 molecule. In an embodiment, the MHC class I molecule is an HLA-A*02:01 molecule. In an embodiment, the MHC class I molecule is an HLA-B*07:02 molecule. In an embodiment, the MHC class I molecule is an HLA-B*15:03 molecule. In an embodiment, the MHC class I molecule is an HLA-B*40:01 molecule. In an embodiment, the MHC class I molecule is an HLA-B*53:01 molecule. In an embodiment, the MHC class I molecule is an HLA-C*02:10 molecule. In an embodiment, the MHC class I molecule is an HLA-C*03:04 molecule. In an embodiment, the MHC class I molecule is an HLA-C*04:01 molecule.

In an embodiment, the TAP (e.g., SEQ ID NOs: 1-39 and 47-62) is non-covalently bound to the MHC class I molecule (i.e., the TAP is loaded into, or non-covalently bound to the peptide binding groove/pocket of the MHC class I molecule). In another embodiment, the TAP is covalently attached/bound to the MHC class I molecule (alpha chain). In such a construct, the TAP and the MHC class I molecule (alpha chain) are produced as a synthetic fusion protein, typically with a short (e.g., 5 to 20 residues, preferably about 8-12, e.g., 10) flexible linker or spacer (e.g., a polyglycine linker). In another aspect, the disclosure provides a nucleic acid encoding a fusion protein comprising a TAP defined herein fused to a MHC class I molecule (alpha chain). In an embodiment, the MHC class I molecule (alpha chain)-peptide complex is multimerized. Accordingly, in another aspect, the present disclosure provides a multimer of MHC class I molecule loaded (covalently or not) with the herein-mentioned TAP. Such multimers may be attached to a tag, for example a fluorescent tag, which allows the detection of the multimers. A great number of strategies have been developed for the production of MHC multimers, including MHC dimers, tetramers, pentamers, octamers, etc. (reviewed in Bakker and Schumacher, Current Opinion in Immunology 2005, 17:428-433). MHC multimers are useful, for example, for the detection and purification of antigen-specific T cells. Thus, in another aspect, the present disclosure provides a method for detecting or purifying (isolating, enriching) CD8⁺ T lymphocytes specific for a TAP defined herein, the method comprising contacting a cell population with a multimer of MHC class I molecule loaded (covalently or not) with the TAP; and detecting or isolating the CD8⁺ T lymphocytes bound by the MHC class I multimers. CD8⁺ T lymphocytes bound by the MHC class I multimers may be isolated using known methods, for example fluorescence activated cell sorting (FACS) or magnetic activated cell sorting (MACS).

In yet another aspect, the present disclosure provides a cell (e.g., a host cell), in an embodiment an isolated cell, comprising the herein-mentioned nucleic acid, vector or plasmid of the disclosure, i.e. a nucleic acid or vector encoding one or more TAPs. In another aspect, the present disclosure provides a cell expressing at its surface an MHC class I molecule (e.g., an MHC class I molecule of one of the alleles disclosed above) bound to or presenting a TAP according to the disclosure. In one embodiment, the host cell is a eukaryotic cell, such as a mammalian cell, preferably a human cell. a cell line or an immortalized cell. In another embodiment, the cell is an antigen-presenting cell (APC). In one embodiment, the host cell is a primary cell, a cell line or an immortalized cell. In another embodiment, the cell is an antigen-presenting cell (APC). Nucleic acids and vectors can be introduced into cells via conventional transformation or transfection techniques. The terms “transformation” and “transfection” refer to techniques for introducing foreign nucleic acid into a host cell, including calcium phosphate or calcium chloride co-precipitation, DEAE-dextran-mediated transfection, lipofection, electroporation, microinjection and viral-mediated transfection. Suitable methods for transforming or transfecting host cells can for example be found in Sambrook et al. (supra), and other laboratory manuals. Methods for introducing nucleic acids into mammalian cells in vivo are also known, and may be used to deliver the vector or plasmid of the disclosure to a subject for gene therapy.

Cells such as APCs can be loaded with one or more TAPs using a variety of methods known in the art. As used herein “loading a cell” with a TAP means that RNA or DNA encoding the TAP, or the TAP, is transfected into the cells or alternatively that the APC is transformed with a nucleic acid encoding the TAP. The cell can also be loaded by contacting the cell with exogenous TAPs that can bind directly to MHC class I molecule present at the cell surface (e.g., peptide-pulsed cells). The TAPs may also be fused to a domain or motif that facilitates its presentation by MHC class I molecules, for example to an endoplasmic reticulum (ER) retrieval signal, a C-terminal Lys-Asp-Glu-Leu sequence (see Wang et al., Eur J Immunol. 2004 December; 34(12):3582-94).

In another aspect, the present disclosure provides a composition or peptide combination/pool comprising any one of, or any combination of, the TAPs defined herein (or a nucleic acid encoding said peptide(s)). In an embodiment, the composition comprises any combination of the TAPs defined herein (any combination of 2, 3, 4, 5, 6, 7, 8, 9, 10 or more TAPs), or a combination of nucleic acids encoding said TAPs). Compositions comprising any combination/sub-combination of the TAPs defined herein are encompassed by the present disclosure. In another embodiment, the combination or pool may comprise one or more known tumor antigens.

Thus, in another aspect, the present disclosure provides a composition comprising any one of, or any combination of, the TAPs defined herein (e.g., SEQ ID NOs: 1-39 and 47-62) and a cell expressing a MHC class I molecule (e.g., a MHC class I molecule of one of the alleles disclosed above). APC for use in the present disclosure are not limited to a particular type of cell and include professional APCs such as dendritic cells (DCs), Langerhans cells, macrophages and B cells, which are known to present proteinaceous antigens on their cell surface so as to be recognized by CD8⁺ T lymphocytes. For example, an APC can be obtained by inducing DCs from peripheral blood monocytes and then contacting (stimulating) the TAPs, either in vitro, ex vivo or in vivo.

APC can also be activated to present a TAP in vivo where one or more of the TAPs of the disclosure are administered to a subject and APCs that present a TAP are induced in the body of the subject. The phrase “inducing an APC” or “stimulating an APC” includes contacting or loading a cell with one or more TAPs, or nucleic acids encoding the TAPs such that the TAPs are presented at its surface by MHC class I molecules. As noted herein, according to the present disclosure, the TAPs may be loaded indirectly for example using longer peptides/polypeptides comprising the sequence of the TAPs (including the native protein), which is then processed (e.g., by proteases) inside the APCs to generate the TAP/MHC class I complexes at the surface of the cells. After loading APCs with TAPs and allowing the APCs to present the TAPs, the APCs can be administered to a subject as a vaccine. For example, the ex vivo administration can include the steps of: (a) collecting APCs from a first subject, (b) contacting/loading the APCs of step (a) with a TAP to form MHC class I/TAP complexes at the surface of the APCs; and (c) administering the peptide-loaded APCs to a second subject in need for treatment.

The first subject and the second subject may be the same subject (e.g., autologous vaccine), or may be different subjects (e.g., allogeneic vaccine). Alternatively, according to the present disclosure, use of a TAP described herein (or a combination thereof) for manufacturing a composition (e.g., a pharmaceutical composition) for inducing antigen-presenting cells is provided. In addition, the present disclosure provides a method or process for manufacturing a pharmaceutical composition for inducing antigen-presenting cells, wherein the method or the process includes the step of admixing or formulating the TAP, or a combination thereof, with a pharmaceutically acceptable carrier. Cells such as APCs expressing a MHC class I molecule (e.g., any of the above-noted HLA molecules) loaded with any one of, or any combination of, the TAPs defined herein, may be used for stimulating/amplifying CD8⁺ T lymphocytes, for example autologous CD8⁺ T lymphocytes. Accordingly, in another aspect, the present disclosure provides a composition comprising any one of, or any combination of, the TAPs defined herein (or a nucleic acid or vector encoding same); a cell expressing an MHC class I molecule and a T lymphocyte, more specifically a CD8⁺ T lymphocyte (e.g., a population of cells comprising CD8⁺ T lymphocytes).

In an embodiment, the composition further comprises a buffer, an excipient, a carrier, a diluent and/or a medium (e.g., a culture medium). In a further embodiment, the buffer, excipient, carrier, diluent and/or medium is/are pharmaceutically acceptable buffer(s), excipient(s), carrier(s), diluent(s) and/or medium (media). As used herein “pharmaceutically acceptable buffer, excipient, carrier, diluent and/or medium” includes any and all solvents, buffers, binders, lubricants, fillers, thickening agents, disintegrants, plasticizers, coatings, barrier layer formulations, lubricants, stabilizing agent, release-delaying agents, dispersion media, coatings, antibacterial and antifungal agents, isotonic agents, and the like that are physiologically compatible, do not interfere with effectiveness of the biological activity of the active ingredient(s) and that are not toxic to the subject. The use of such media and agents for pharmaceutically active substances is well known in the art (Rowe et al., Handbook of pharmaceutical excipients, 2003, 4^{th} edition, Pharmaceutical Press, London UK). Except insofar as any conventional media or agent is incompatible with the active compound (peptides, cells), use thereof in the compositions of the disclosure is contemplated. In an embodiment, the buffer, excipient, carrier and/or medium is a non-naturally occurring buffer, excipient, carrier and/or medium. In an embodiment, one or more of the TAPs defined herein, or the nucleic acids (e.g., mRNAs) encoding said one or more TAPs, are comprised within or complexed to a lipid vesicle or liposome, e.g., a cationic liposome (see, e.g., Vitor M T et al., Recent Pat Drug Deliv Formul. 2013 August; 7(2):99-110) or suitable other carriers.

In another aspect, the present disclosure provides a composition comprising one of more of the any one of, or any combination of, the TAPs defined herein (e.g., SEQ ID NOs: 1-39 and 47-62) (or a nucleic acid encoding said peptide(s)), and a buffer, an excipient, a carrier, a diluent and/or a medium. For compositions comprising cells (e.g., APCs, T lymphocytes), the composition comprises a suitable medium that allows the maintenance of viable cells. Representative examples of such media include saline solution, Earl's Balanced Salt Solution (Life Technologies®) or PlasmaLyte® (Baxter International®). In an embodiment, the composition (e.g., pharmaceutical composition) is an “immunogenic composition”, “vaccine composition” or “vaccine”. The term “Immunogenic composition”, “vaccine composition” or “vaccine” as used herein refers to a composition or formulation comprising one or more TAPs or vaccine vector and which is capable of inducing an immune response against the one or more TAPs present therein when administered to a subject. Vaccination methods for inducing an immune response in a mammal comprise use of a vaccine or vaccine vector to be administered by any conventional route known in the vaccine field, e.g., via a mucosal (e.g., ocular, intranasal, pulmonary, oral, gastric, intestinal, rectal, vaginal, or urinary tract) surface, via a parenteral (e.g., subcutaneous, intradermal, intramuscular, intravenous, or intraperitoneal) route, or topical administration (e.g., via a transdermal delivery system such as a patch). In an embodiment, the TAP (or a combination thereof) is conjugated to a carrier protein (conjugate vaccine) to increase the immunogenicity of the TAP(s). The present disclosure thus provides a composition (conjugate) comprising a TAP (or a combination thereof), or a nucleic acid encoding the TAP or combination thereof, and a carrier protein. For example, the TAP(s) or nucleic acid(s) may be conjugated or complexed to a Toll-like receptor (TLR) ligand (see, e.g., Zom et al., Adv Immunol. 2012, 114: 177-201) or polymers/dendrimers (see, e.g., Liu et al., Biomacromolecules. 2013 Aug. 12; 14(8):2798-806). In an embodiment, the immunogenic composition or vaccine further comprises an adjuvant. “Adjuvant” refers to a substance which, when added to an immunogenic agent such as an antigen (TAPs, nucleic acids and/or cells according to the present disclosure), nonspecifically enhances or potentiates an immune response to the agent in the host upon exposure to the mixture. Examples of adjuvants currently used in the field of vaccines include (1) mineral salts (aluminum salts such as aluminum phosphate and aluminum hydroxide, calcium phosphate gels), squalene, (2) oil-based adjuvants such as oil emulsions and surfactant based formulations, e.g., MF59 (microfluidised detergent stabilised oil-in-water emulsion), QS21 (purified saponin), AS02 [SBAS2] (oil-in-water emulsion+MPL+QS-21), (3) particulate adjuvants, e.g., virosomes (unilamellar liposomal vehicles incorporating influenza haemagglutinin), AS04 ([SBAS4]aluminum salt with MPL), ISCOMS (structured complex of saponins and lipids), polylactide co-glycolide (PLG), (4) microbial derivatives (natural and synthetic), e.g., monophosphoryl lipid A (MPL), Detox (MPL+M. phlei cell wall skeleton), AGP [RC-529] (synthetic acylated monosaccharide), DC_Chol (lipoidal immunostimulators able to self-organize into liposomes), OM-174 (lipid A derivative), CpG motifs (synthetic oligonucleotides containing immunostimulatory CpG motifs), modified LT and CT (genetically modified bacterial toxins to provide non-toxic adjuvant effects), (5) endogenous human immunomodulators, e.g., hGM-CSF or hIL-12 (cytokines that can be administered either as protein or plasmid encoded), Immudaptin (C3d tandem array) and/or (6) inert vehicles, such as gold particles, and the like.

In an embodiment, the TAP(s) (e.g., SEQ ID NOs: 1-39 and 47-62) or composition comprising same is/are in lyophilized form. In another embodiment, the TAP(s) or composition comprising same is/are in a liquid composition. In a further embodiment, the TAP(s) is/are at a concentration of about 0.01 μg/mL to about 100 μg/mL in the composition. In further embodiments, the TAP(s) is/are at a concentration of about 0.2 μg/mL to about 50 μg/mL, about 0.5 μg/mL to about 10, 20, 30, 40 or 50 μg/mL, about 1 μg/mL to about 10 μg/mL, or about 2 μg/mL, in the composition.

As noted herein, cells such as APCs that express an MHC class I molecule loaded with or bound to any one of, or any combination of, the TAPs defined herein, may be used for stimulating/amplifying CD8⁺ T lymphocytes in vivo or ex vivo. Accordingly, in another aspect, the present disclosure provides T cell receptor (TCR) molecules capable of interacting with or binding the herein-mentioned MHC class I molecule/TAP complex, and nucleic acid molecules encoding such TCR molecules, and vectors comprising such nucleic acid molecules. A TCR according to the present disclosure is capable of specifically interacting with or binding a TAP loaded on, or presented by, an MHC class I molecule, preferably at the surface of a living cell in vitro or in vivo.

The term TCR as used herein refers to an immunoglobulin superfamily member having a variable binding domain, a constant domain, a transmembrane region, and a short cytoplasmic tail; see, e.g., Janeway et al, Immunobiology: The Immune System in Health and Disease, 3rd Ed., Current Biology Publications, p. 4:33, 1997) capable of specifically binding to an antigen peptide bound to a MHC receptor. A TCR can be found on the surface of a cell and generally is comprised of a heterodimer having α and β chains (also known as TCRα and TCRβ, respectively). Like immunoglobulins, the extracellular portion of TCR chains (e.g., α-chain, β-chain) contain two immunoglobulin regions, a variable region (e.g., TCR variable α region or Vα and TCR variable β region or Vβ; typically amino acids 1 to 116 based on Rabat numbering at the N-terminus), and one constant region (e.g., TCR constant domain α or Cα and typically amino acids 117 to 259 based on Rabat, TCR constant domain β or Cβ, typically amino acids 117 to 295 based on Rabat) adjacent to the cell membrane. Also, like immunoglobulins, the variable domains contain complementary determining regions (CDRs. 3 in each chain) separated by framework regions (FRs). In certain embodiments, a TCR is found on the surface of T cells (or T lymphocytes) and associates with the CD3 complex.

A TCR and in particular nucleic acids encoding a TCR of the disclosure may for instance be applied to genetically transform/modify T lymphocytes (e.g., CD8⁺ T lymphocytes) or other types of lymphocytes generating new T lymphocyte clones that specifically recognize an MHC class I/TAP complex. In a particular embodiment, T lymphocytes (e.g., CD8⁺ T lymphocytes) obtained from a patient are transformed to express one or more TCRs that recognize a TAP and the transformed cells are administered to the patient (autologous cell transfusion). In a particular embodiment, T lymphocytes (e.g., CD8⁺ T lymphocytes) obtained from a donor are transformed to express one or more TCRs that recognize a TAP and the transformed cells are administered to a recipient (allogenic cell transfusion). In another embodiment, the disclosure provides a T lymphocyte e.g., a CD8⁺ T lymphocyte transformed/transfected by a vector or plasmid encoding a TAP-specific TCR. In a further embodiment the disclosure provides a method of treating a patient with autologous or allogenic cells transformed with a TAP-specific TCR. In certain embodiments, TCRs are expressed in primary T cells (e.g., cytotoxic T cells) by replacing an endogenous locus, e.g., an endogenous TRAC and/or TRBC locus, using, e.g., CRISPR, TALEN, zinc finger, or other targeted disruption systems.

In an embodiment, the anti-CSC TCR according to the present disclosure comprises a TCRbeta (β) chain comprising a complementary determining region 3 (CDR3) comprising one of the amino acid sequences set forth in Table 3B (SEQ ID NO:73-84).

In another embodiment, the present disclosure provides a nucleic acid encoding the above-noted TCR. In a further embodiment, the nucleic acid is present in a vector, such as the vectors described above.

In yet a further embodiment the use of a CSC tumor antigen-specific TCR in the manufacture of autologous or allogenic cells for the treating of cancer (e.g., a cancer associated with the presence of CSCs such as a poorly differentiated cancer) is provided.

In some embodiments, patients treated with the compositions (e.g., pharmaceutical compositions) of the disclosure are treated prior to or following treatment with an anti-tumor agent and/or immunotherapy (e.g., CAR therapy). Compositions of the disclosure include: allogenic T lymphocytes (e.g., CD8⁺ T lymphocyte) activated ex vivo against a TAP; allogenic or autologous APC vaccines loaded with a TAP; TAP vaccines and allogenic or autologous T lymphocytes (e.g., CD8⁺ T lymphocyte) or lymphocytes transformed with a tumor antigen-specific TCR. The method to provide T lymphocyte clones capable of recognizing a TAP according to the disclosure may be generated for and can be specifically targeted to tumor cells expressing the TAP in a subject (e.g., graft recipient), for example an ASCT and/or donor lymphocyte infusion (DLI) recipient. Hence the disclosure provides a CD8⁺ T lymphocyte encoding and expressing a T cell receptor capable of specifically recognizing or binding a TAP/MHC class I molecule complex. Said T lymphocyte (e.g., CD8⁺ T lymphocyte) may be a recombinant (engineered) or a naturally selected T lymphocyte. This specification thus provides at least two methods for producing CD8⁺ T lymphocytes of the disclosure, comprising the step of bringing undifferentiated lymphocytes into contact with a TAP/MHC class I molecule complex (typically expressed at the surface of cells, such as APCs) under conditions conducive of triggering T cell activation and expansion, which may be done in vitro or in vivo (i.e., in a patient administered with a APC vaccine wherein the APC is loaded with a TAP or in a patient treated with a TAP vaccine). Using a combination or pool of TAPs bound to MHC class I molecules, it is possible to generate a population CD8⁺ T lymphocytes capable of recognizing a plurality of TAPs. Alternatively, tumor antigen-specific or targeted T lymphocytes may be produced/generated in vitro or ex vivo by cloning one or more nucleic acids (genes) encoding a TCR (more specifically the alpha and beta chains) that specifically binds to a MHC class I molecule/TAP complex (i.e. engineered or recombinant CD8⁺ T lymphocytes). Nucleic acids encoding a TAP-specific TCR of the disclosure, may be obtained using methods known in the art from a T lymphocyte activated against a TAP ex vivo (e.g., with an APC loaded with a TAP); or from an individual exhibiting an immune response against peptide/MHC molecule complex. TAP-specific TCRs of the disclosure may be recombinantly expressed in a host cell and/or a host lymphocyte obtained from a graft recipient or graft donor, and optionally differentiated in vitro to provide cytotoxic T lymphocytes (CTLs). The nucleic acid(s) (transgene(s)) encoding the TCR alpha and beta chains may be introduced into a T cells (e.g., from a subject to be treated or another individual) using any suitable methods such as transfection (e.g., electroporation) or transduction (e.g., using viral vector). The engineered CD8⁺ T lymphocytes expressing a TCR specific for a TAP may be expanded in vitro using well known culturing methods.

The present disclosure provides methods for making the immune effector cells which express the TCRs as described herein. In one embodiment, the method comprises transfecting or transducing immune effector cells, e.g., immune effector cells isolated from a subject, such as a subject having a colorectal cancer (e.g., colon cancer, rectal cancer), such that the immune effector cells express one or more TCR as described herein. In certain embodiments, the immune effector cells are isolated from an individual and genetically modified without further manipulation in vitro. Such cells can then be directly re-administered into the individual. In further embodiments, the immune effector cells are first activated and stimulated to proliferate in vitro prior to being genetically modified to express a TCR. In this regard, the immune effector cells may be cultured before or after being genetically modified (i.e., transduced or transfected to express a TCR as described herein).

Prior to in vitro manipulation or genetic modification of the immune effector cells described herein, the source of cells may be obtained from a subject. In particular, the immune effector cells for use with the TCRs as described herein comprise T cells. T cells can be obtained from a number of sources, including peripheral blood mononuclear cells (PBMCs), bone marrow, lymph nodes tissue, cord blood, thymus issue, tissue from a site of infection, ascites, pleural effusion, spleen tissue, and tumors. In certain embodiments, T cell can be obtained from a unit of blood collected from the subject using any number of techniques known to the skilled person, such as FICOLL™ separation. In one embodiment, cells from the circulating blood of an individual are obtained by apheresis. The apheresis product typically contains lymphocytes, including T cells, monocytes, granulocyte, B cells, other nucleated white blood cells, red blood cells, and platelets. In one embodiment, the cells collected by apheresis may be washed to remove the plasma fraction and to place the cells in an appropriate buffer or media for subsequent processing. In one embodiment of the invention, the cells are washed with PBS. In an alternative embodiment, the washed solution lacks calcium and may lack magnesium or may lack many if not all divalent cations. As would be appreciated by those of ordinary skill in the art, a washing step may be accomplished by methods known to those in the art, such as by using a semi-automated flow-through centrifuge. After washing, the cells may be resuspended in a variety of biocompatible buffers or other saline solution with or without buffer. In certain embodiments, the undesirable components of the apheresis sample may be removed in the cell directly resuspended culture media. In certain embodiments, T cells are isolated from peripheral blood mononuclear cells (PBMCs) by lysing the red blood cells and depleting the monocytes, for example, by centrifugation through a PERCOLL™ gradient. A specific subpopulation of T cells, such as CD28+, CD4+, CD8+, CD45RA+, and CD45RO+ T cells, can be further isolated by positive or negative selection techniques. For example, enrichment of a T cell population by negative selection can be accomplished with a combination of antibodies directed to surface markers unique to the negatively selected cells. One method for use herein is cell sorting and/or selection via negative magnetic immunoadherence or flow cytometry that uses a cocktail of monoclonal antibodies directed to cell surface markers present on the cells negatively selected. For example, to enrich for CD8+ cells by negative selection, a monoclonal antibody cocktail typically includes antibodies to CD14, CD20, CD11b, CD16, HLA-DR, and CD4. Flow cytometry and cell sorting may also be used to isolate cell populations of interest for use in the present disclosure. PBMC may be used directly for genetic modification with the TCRs using methods as described herein. In certain embodiments, after isolation of PBMC, T lymphocytes are further isolated and in certain embodiments, both cytotoxic and helper T lymphocytes can be sorted into naive, memory, and effector T cell subpopulations either before or after genetic modification and/or expansion.

The present disclosure provides isolated immune cells such as CD8⁺ T lymphocytes that are specifically induced, activated and/or amplified (expanded) by a TAP (i.e., a TAP bound to MHC class I molecules expressed at the surface of cell), or a combination of TAPs. The present disclosure also provides a composition comprising CD8⁺ T lymphocytes capable of recognizing a TAP, or a combination thereof, according to the disclosure (i.e., one or more TAPs bound to MHC class I molecules) and said TAP(s). In another aspect, the present disclosure provides a cell population or cell culture (e.g., a CD8⁺ T lymphocyte population) enriched in CD8⁺ T lymphocytes that specifically recognize one or more MHC class I molecule/TAP complex(es) as described herein. Such enriched population may be obtained by performing an ex vivo expansion of specific T lymphocytes using cells such as APCs that express MHC class I molecules loaded with (e.g., presenting) one or more of the TAPs disclosed herein. “Enriched” as used herein means that the proportion of tumor antigen-specific CD8⁺ T lymphocytes in the population is significantly higher relative to a native population of cells, i.e., which has not been subjected to a step of ex vivo-expansion of specific T lymphocytes. In a further embodiment, the proportion of TAP-specific CD8⁺ T lymphocytes in the cell population is at least about 0.5%, for example at least about 1%, 1.5%, 2% or 3%. In some embodiments, the proportion of TAP-specific CD8⁺ T lymphocytes in the cell population is about 0.5 to about 10%, about 0.5 to about 8%, about 0.5 to about 5%, about 0.5 to about 4%, about 0.5 to about 3%, about 1% to about 5%, about 1% to about 4%, about 1% to about 3%, about 2% to about 5%, about 2% to about 4%, about 2% to about 3%, about 3% to about 5% or about 3% to about 4%. Such cell population or culture (e.g., a CD8⁺ T lymphocyte population) enriched in CD8⁺ T lymphocytes that specifically recognizes one or more MHC class I molecule/peptide (TAP) complex(es) of interest may be used in tumor antigen-based cancer immunotherapy, as detailed below. In some embodiments, the population of TAP-specific CD8⁺ T lymphocytes is further enriched, for example using affinity-based systems such as multimers of MHC class I molecule loaded (covalently or not) with the TAP(s) defined herein. Thus, the present disclosure provides a purified or isolated population of TAP-specific CD8⁺ T lymphocytes, e.g., in which the proportion of TAP-specific CD8⁺ T lymphocytes is at least about 50%, 60%, 70%, 80%, 85%, 90%, 95%, 96%, 97%, 98%, 99% or 100%.

In another aspect, the present disclosure provides an antibody or an antigen-binding fragment thereof that specifically binds to a complex comprising a TAP as described herein bound to an HLA molecule, such as the HLA molecules defined herein. Such antibodies are commonly referred to as TCR-like antibodies. The term “antibody or antigen-binding fragment thereof” as used herein refers to any type of antibody/antibody fragment including monoclonal antibodies (including full-length monoclonal antibodies), polyclonal antibodies, multispecific antibodies, humanized antibodies, CDR-grafted antibodies, chimeric antibodies and antibody fragments so long as they exhibit the desired antigenic specificity/binding activity. Antibody fragments comprise a portion of a full-length antibody, generally an antigen binding or variable region thereof. Examples of antibody fragments include Fab, Fab′, F(ab′)₂, and Fv fragments, diabodies, linear antibodies, single-chain antibody molecules (e.g., single-chain Fv, scFv), single domain antibodies (e.g., from camelids), shark NAR single domain antibodies, and multispecific antibodies formed from antibody fragments, single-chain diabodies (scDbs), bispecific T cell engagers (BiTEs), dual affinity retargeting molecules (DARTs), bivalent scFv-Fcs, and trivalent scFv-Fcs. Antibody fragments can also refer to binding moieties comprising CDRs or antigen binding domains including, but not limited to, V_{H} regions (V_{H}, V_{H}-V_{H}), anticalins, PepBodies, antibody-T-cell epitope fusions (Troybodies) or Peptibodies. In an embodiment, the antibody or antigen-binding fragment thereof is a single-chain antibody, preferably a single-chain Fv (scFv). In an embodiment, the antibody or antigen-binding fragment thereof comprises at least one constant domain, e.g., a constant domain of a light and/or heavy chain, or a fragment thereof. In a further embodiment, the antibody or antigen-binding fragment thereof comprises a Fragment crystallizable (Fc) fragment of the constant heavy chain of an antibody. In an embodiment, the antibody or antigen-binding fragment is a scFv comprising a Fc fragment (scFV-Fc). In an embodiment, the scFv component is connected to the Fc fragment by a linker, for example a hinge. The presence of an Fc region is useful to induce a Complement-dependent cytotoxicity (CDC) or antibody-dependent cellular cytotoxicity (ADCC) response against a tumor cell.

In an embodiment, the antibody or antigen-binding fragment thereof is a multispecific antibody or an antigen-binding fragment thereof, such as a bispecific antibody or an antigen-binding fragment thereof, wherein at least one of the antigen-binding domains of the multispecific antibody or antibody fragment recognize(s) a complex comprising a TAP as described herein bound to an HLA molecule. In an embodiment, at least one of the antigen-binding domains of the multispecific antibody or antibody fragment recognize(s) an immune cell effector molecule. The term “immune cell effector molecule” refers to a molecule (e.g., protein) expressed by an immune cell and whose engagement by the multispecific antibody or antibody fragment leads to activation of the immune cells. Examples of immune cell effector molecules include the CD3 signaling complex in T cells such as CD8 T cells and the various activating receptors on NK cells (NKG2D, KIR2DS, NKp44, etc.). In a further embodiment, at least one of the antigen-binding domains of the multispecific antibody or antibody fragment recognize(s) and engage(s) the CD3 signaling complex in T cells (e.g., anti-CD3). In a further embodiment, the multispecific antibody or antibody fragment is a single-chain diabody (scDb). In a further embodiment, the scDb comprises a first antibody fragment (e.g., scFv) that binds to a complex comprising a TAP as described herein bound to an HLA molecule and a second antibody fragment (e.g., scFv) that binds to and engages an immune cell effector molecule, such as the CD3 signaling complex in T cells (e.g., anti-CD3 scFv). Such constructs may be used for example to induce the cytotoxic T cell-mediated killing of tumor cells expressing the tumor antigen/MHC complex recognized by the multispecific antibody or antibody fragment. Antibodies or antigen-binding fragments thereof may also be used as a chimeric antigen receptor (CAR) to produce CAR T cells, CAR NK cells, etc. CAR combines a ligand-binding domain (e.g. antibody or antibody fragment) that provides specificity for a desired antigen (e.g., MHC/TAP complex) with an activating intracellular domain (or signal transducing domain) portion, such as a T cell or NK cell activating domain, providing a primary activation signal. Antigen-binding fragments of antibodies, and more particularly scFv, capable of binding to molecules expressed by tumor cells are commonly used as ligand-binding domains in CAR. Thus, in another aspect, the present disclosure provides a host cell, preferably an immune cell such as a T cell or NK cell, expressing the antibody or antibody fragment (e.g., scFv) described herein.

The present disclosure further relates to a pharmaceutical composition or vaccine comprising the above-noted immune cell (CD8⁺ T lymphocytes, CAR T cell) or population of TAP-specific CD8⁺ T lymphocytes. Such pharmaceutical composition or vaccine may comprise one or more pharmaceutically acceptable excipients and/or adjuvants, as described above.

The present disclosure further relates to the use of any TAP (e.g., SEQ ID NOs: 1-39 and 47-62, preferably SEQ ID NOs: 1-39), nucleic acid, expression vector, T cell receptor, antibody/antibody fragment, cell (e.g., T lymphocyte, APC, CAR T cell), and/or composition according to the present disclosure, or any combination thereof, as a medicament or in the manufacture of a medicament. In an embodiment, the medicament is for the treatment of cancer, e.g., cancer vaccine. The present disclosure relates to any TAP, nucleic acid, expression vector, T cell receptor, antibody/antibody fragment, cell (e.g., T lymphocyte, APC), and/or composition (e.g., vaccine composition) according to the present disclosure, or any combination thereof, for use in the treatment of cancer e.g., as a cancer vaccine. The TAP sequences identified herein may be used for the production of synthetic peptides to be used i) for in vitro priming and expansion of tumor antigen-specific T cells to be injected into tumor patients and/or ii) as vaccines to induce or boost the anti-tumor T cell response in cancer patients, such as patients suffering from cancers associated with the presence of cancer stem cells, e.g., poorly differentiated cancers.

The term “cancer stem cells” (CSCs) as used herein refers to a subpopulation of cancer cells, found within solid tumors or hematological cancers, that drive tumor initiation and possess characteristics associated with normal stem cells, specifically the ability of self-renewal and differentiation into multiple tumor cell types. CSCs have been shown to exhibit resistance to chemotherapy (multidrug resistance) and radiotherapy, and are associated with cancer relapse and metastasis. Cancer stem cells encompass cells expressing certain markers. Examples of markers of CSCs in various types of cancers are depicted in the table below (see, e.g., Walcher et al., “Cancer Stem Cells—Origins and Biomarkers: Perspectives for Targeted Personalized Therapies”, Front Immunol. 2020; 11: 1280; Suster et al., “Presence and role of stem cells in ovarian cancer”, World J Stem Cells. 2019 Jul. 26; 11(7): 383-397).

Example of CSC markers in different types of cancers

| Cancer type | CSC markers |
|---|---|
| Lung cancer | Cell surface: CD44 (and variants), CD87, CD90, CD133, CD166, EpCAM |
| | Intracellular: ALDH, Nanog, Oct-3/4 |
| CML | Cell surface: CD25, CD26, CD33, CD36, CD117, CD123, IL1RAP |
| | Intracellular: JAK/STAT, Wnt/β-catenin, FOXO, Hedgehog/Smo/Gli2 |
| Breast | Cell surface: CD24, CD29, CD44 (and variants), CD49f, CD61, CD70, |
| | CD90, CD133, CXCR4, EpCAM, LGR5, ProC-R |
| | Intracellular: ALDH, BMI-1, Nanog, Notch, Oct-3/4, Sox2, Wnt/β-catenin |
| Gastric | Cell surface: CD24, CD44 (and variants), CD90, CD133, CXCR4, |
| | EpCAM, LGR5, LINGO2 |
| | Intracellular: ALDH, Letm1, Musashi2, Nanog, Oct-3/4, Sox2 |
| Liver | Cell surface: CD24, CD44, CD90, CD133, EpCAM |
| | Intracellular: AFP, Nanog, Notch, Oct-3/4, Sox2, Wnt/β-catenin |
| Colorectal | Cell surface: CD24, CD44, CD133, CD166, EpCAM, LGR5 |
| | Intracellular: ALDH, Letm1, Nanog, Oct-3/4, Sall4, Sox2 |
| AML | Cell surface: CD33, CD123, CLL-1, TIM3 |
| | Intracellular: ALDH, Nanog, Oct-3/4, Sox2 |
| Ovarian | Cell surface: CD24, CD44, CD117, CD133, EpCAM, ROR1 |
| | Intracellular: ALDH, Nanog, Ssea-4, Oct-4, Sox2, MYC |
| Melanoma | Cell surface: Nestin, CD133 |
| Glioma | Cell surface: Nestin, CD133 |

CSCs are also known to express or overexpress multidrug resistance (MDR) proteins (MRPs). MRPs are members of the C family of a group of proteins named ATP-binding cassette (ABC) transporters that efflux a wide spectrum of anticancer drugs against the concentration gradient using ATP-driven energy. The most common MRPs are ABC subfamily C member 1 (ABCC1/MRP1), ABC subfamily C member 2 (ABCC2/MRP2), ABC subfamily C member 3 (ABCC3/MRP3), ABC subfamily C member 4 (ABCC4/MRP4), ABC subfamily C member 5 (ABCC5/MRP5), ABC subfamily C member 6 (ABCC6/MRP6), ABC subfamily C member 10 (ABCC10/MRP7), ABC subfamily C member 11 (ABCC11/MRP8), ABC subfamily C member 12 (ABCC12/MRP9), ABC subfamily B member 1 (ABCB1, also known as P-glycoprotein (P-gp)), ABC subfamily B member 5 (ABCB5) and ABC subfamily G member 2 (ABCG2).

Thus, in an embodiment, the methods and uses defined herein aimed at killing CSCs expressing one or more of the markers listed above.

The cancer may be a tumor affecting any tissue or organ that comprises CSCs, such as heart sarcoma, lung cancer, small cell lung cancer (SCLC), non-small cell lung cancer (NSCLC), bronchogenic carcinoma (squamous cell, undifferentiated small cell, undifferentiated large cell, adenocarcinoma), alveolar (bronchiolar) carcinoma, bronchial adenoma, sarcoma (e.g., Ewing's sarcoma, Karposi's sarcoma), lymphoma, chondromatous hamartoma, mesothelioma; cancer of the gastrointestinal system, for example, esophagus (squamous cell carcinoma, adenocarcinoma, leiomyosarcoma, lymphoma), stomach (carcinoma, lymphoma, leiomyosarcoma), gastric, pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); cancer of the genitourinary tract, for example, kidney cancer (adenocarcinoma, Wilm's tumor [nephroblastoma], lymphoma, leukemia), bladder and/or urethra cancer (squamous cell carcinoma, transitional cell carcinoma, adenocarcinoma), prostate cancer (adenocarcinoma, sarcoma), testis cancer (seminoma, teratoma, embryonal carcinoma, teratocarcinoma, choriocarcinoma, sarcoma, interstitial cell carcinoma, fibroma, fibroadenoma, adenomatoid tumors, lipoma); liver cancer, for example, hepatoma (hepatocellular carcinoma, HCC), cholangiocarcinoma, hepatoblastoma, angiosarcoma, hepatocellular adenoma, hemangioma, pancreatic endocrine tumors (such as pheochromocytoma, insulinoma, vasoactive intestinal peptide tumor, islet cell tumor and glucagonoma); bone cancer, for example, osteogenic sarcoma (osteosarcoma), fibrosarcoma, malignant fibrous histiocytoma, chondrosarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; cancer of the nervous system, for example, neoplasms of the central nervous system (CNS), primary CNS lymphoma, skull cancer (osteoma, hemangioma, granuloma, xanthoma, osteitis deformans), meninges (meningioma, meningiosarcoma, gliomatosis), brain cancer (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); cancer of the reproductive system, for example, gynecological cancer, uterine cancer (endometrial carcinoma), cervical cancer (cervical carcinoma, pre-tumor cervical dysplasia), ovarian cancer (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulvar cancer (squamous cell carcinoma, intraepithelial carcinoma, adenocarcinoma, fibrosarcoma, melanoma), vaginal cancer (clear cell carcinoma, squamous cell carcinoma, botryoid sarcoma (embryonal rhabdomyosarcoma), fallopian tube cancer (carcinoma); placenta cancer, penile cancer, prostate cancer, testicular cancer; cancer of the hematologic system, for example, blood cancer (acute myeloid leukemia (AML), chronic myeloid leukemia (CML), acute lymphoblastic leukemia (ALL), chronic lymphocytic leukemia (CLL), myeloproliferative diseases, multiple myeloma, myelodysplastic syndrome), Hodgkin's disease, non-Hodgkin's lymphoma [malignant lymphoma]; cancer of the oral cavity, for example, lip cancer, tongue cancer, gum cancer, palate cancer, oropharynx cancer, nasopharynx cancer, sinus cancer; skin cancer, for example, malignant melanoma, cutaneous melanoma, basal cell carcinoma, squamous cell carcinoma, Karposi's sarcoma, moles dysplastic nevi, lipoma, angioma, dermatofibroma, and keloids; adrenal gland cancer: neuroblastoma; and cancers of other tissues including connective and soft tissue, retroperitoneum and peritoneum, eye cancer, intraocular melanoma, and adnexa, breast cancer (e.g., ductal breast cancer), head or/and neck cancer (head and neck squamous cell carcinoma), anal cancer, thyroid cancer, parathyroid cancer; secondary and unspecified malignant neoplasm of lymph nodes, secondary malignant neoplasm of respiratory and digestive systems and secondary malignant neoplasm of other sites. In an embodiment, the cancer is leukemia (e.g., AML), brain cancer (e.g., glioblastoma), breast cancer, colon cancer, liver cancer (e.g., hepatocellular carcinoma), ovarian cancer, pancreatic cancer, prostate cancer, skin cancer (e.g., melanoma), or myeloma (e.g., multiple myeloma).

In an embodiment, the methods and uses defined herein aimed at treating poor prognosis cancers. The term “poor prognosis cancer” as used herein refers to a subtype of a given cancer that is associated with lower survival rate (e.g., 5-year or 10-year survival rate) relative to other subtype(s) of the same cancer. Poor prognosis cancer is generally associated with specific characteristics of the cancer subtype, for example the presence of certain mutations, chromosomal abnormalities, etc., that renders them more resistant to treatment. Poor prognosis is also associated with cancers diagnosed at a later stage (e.g., with distant metastasis). Also, as noted above, high CSC frequency has been shown to correlate with poor response to treatment and lower survival in several cancers. For example, for breast cancer, triple-negative breast cancer (TNBC) is considered a poor prognosis breast cancer as it is associated with a lower 5-year relative survival rate relative to other breast cancer subtypes. Also, high levels of circulating cancer stem-like cells (cCSCs) have been associated with an inferior tumor response rate to chemotherapy and lower overall and progression-free survival in breast cancer patients (Lee, C H et al., BMC Cancer 19, 1167 (2019)). For ovarian cancer, invasive epithelial ovarian cancer and fallopian tube cancer are generally associated with a lower 5-year relative survival rate relative to ovarian stromal tumors and germ cell tumors. The 5-year overall survival rate of pancreatic cancer is very low (about 3%), which is partly because more than half of the patients are diagnosed at an advanced stage. Diagnosis of pancreatic cancer at stage III/IV (with distant metastasis) is associated with very poor prognosis. Similarly, for prostate cancer, diagnosis at stage IV (with distant metastasis) is associated with poor prognosis (5-year relative survival rate of less than 30% compared to at least 80-85% for diagnosis at stages I-III). For lung cancer, small cell lung cancer is associated with particularly poor prognosis, especially when diagnosed at a later stage (e.g., with regional or distant metastasis). Non-small cell lung cancer diagnosed at a later stage (e.g., with distant metastasis) is also associated with poor prognosis. In colorectal cancer, mucinous adenocarcinomas (characterized by the presence of abundant extracellular mucin) have been associated with reduced response to chemotherapy and poor prognosis. Peritoneal involvement and BRAF mutations also constitute poor prognosis markers for colorectal cancer. For kidney cancer, clear cell RCC is associated with worse outcomes (e.g., lower 5-year relative survival rate) than papillary RCC. In skin cancer, thicker tumors, nodal involvement and diagnosis at a later stage (e.g., with regional or distant metastasis) are associated with lower survival in melanoma. Expression of Nestin and CD133 has been associated with poor outcome in melanoma and glioma.

In an embodiment, the poor prognosis cancer is a stage III/IV cancer. In another embodiment, the poor prognosis cancer is a cancer with a high number or frequency of CSCs, i.e. a number or frequency of CSCs that is higher than the average number or frequency of CSCs in the same type of cancer (e.g., ovarian cancer, breast cancer). In an embodiment, the number or frequency of CSCs is at least 5%, 10%, 20%, 30%, 40%, 50%, 60%, 70%, 80%, 90%, 100% (2-fold), 200% (3-fold), 300% (4-fold) or 400% (5-fold) than the average number or frequency of CSCs in the same type of cancer.

In an embodiment, the poor prognosis cancer is a cancer having a 5-year relative survival rate of less than 60%. In an embodiment, the poor prognosis cancer is a cancer having a 5-year relative survival rate of less than 50%. In an embodiment, the poor prognosis cancer is a cancer having a 5-year relative survival rate of less than 40%. In an embodiment, the poor prognosis cancer is a cancer having a 5-year relative survival rate of less than 30%. In an embodiment, the poor prognosis cancer is a cancer having a 5-year relative survival rate of less than 20%. In an embodiment, the poor prognosis cancer is a cancer having a 5-year relative survival rate of less than 10%. In an embodiment, the poor prognosis cancer is a cancer having a 5-year relative survival rate of less than 5%.

In another aspect, the present disclosure provides the use of a TAP described herein (e.g., SEQ ID NOs: 1-39 and 47-62, preferably SEQ ID NOs: 1-39), or a combination thereof (e.g., a peptide pool), as a vaccine for treating cancer, such as cancers associated with the presence of CSCs, in a subject. The present disclosure also provides the TAP described herein, or a combination thereof (e.g., a peptide pool), for use as a vaccine for treating cancer, such as a lymphoblastic leukemia, in a subject. In an embodiment, the subject is a recipient of TAP-specific CD8⁺ T lymphocytes. Accordingly, in another aspect, the present disclosure provides a method of treating cancer (e.g., of reducing the number of tumor cells, killing tumor cells), said method comprising administering (infusing) to a subject in need thereof an effective amount of CD8⁺ T lymphocytes recognizing (i.e., expressing a TCR that binds) one or more MHC class I molecule/TAP complexes (expressed at the surface of a cell such as an APC). In an embodiment, the method further comprises administering an effective amount of the TAP, or a combination thereof, and/or a cell (e.g., an APC such as a dendritic cell) expressing MHC class I molecule(s) loaded with the TAP(s), to said subject after administration/infusion of said CD8⁺ T lymphocytes. In yet a further embodiment, the method comprises administering to a subject in need thereof a therapeutically effective amount of a dendritic cell loaded with one or more TAPs. In yet a further embodiment the method comprises administering to a patient in need thereof a therapeutically effective amount of an allogenic or autologous cell that expresses a recombinant TCR that binds to a TAP presented by an MHC class I molecule.

In another aspect, the present disclosure provides the use of CD8⁺ T lymphocytes that recognize one or more MHC class I molecules loaded with (presenting) a TAP, or a combination thereof, for treating cancer (e.g., of reducing the number of tumor cells, killing tumor cells) in a subject. In another aspect, the present disclosure provides the use of CD8⁺ T lymphocytes that recognize one or more MHC class I molecules loaded with (presenting) a TAP, or a combination thereof, for the preparation/manufacture of a medicament for treating cancer (e.g., for reducing the number of tumor cells, killing tumor cells), such as a lymphoblastic leukemia, in a subject. In another aspect, the present disclosure provides CD8⁺ T lymphocytes (cytotoxic T lymphocytes) that recognize one or more MHC class I molecule(s) loaded with (presenting) a TAP, or a combination thereof, for use in the treatment of cancer (e.g., for reducing the number of tumor cells, killing tumor cells), such as a lymphoblastic leukemia, in a subject. In a further embodiment, the use further comprises the use of an effective amount of a TAP (or a combination thereof), and/or of a cell (e.g., an APC) that expresses one or more MHC class I molecule(s) loaded with (presenting) a TAP, after the use of said TAP-specific CD8⁺ T lymphocytes.

The present disclosure also provides a method of generating an immune response against tumor cells expressing human class I MHC molecules loaded with any of the TAP disclosed herein (e.g., SEQ ID NOs: 1-39 and 47-62, preferably SEQ ID NOs: 1-39) or combination thereof in a subject, the method comprising administering cytotoxic T lymphocytes that specifically recognizes the class I MHC molecules loaded with the TAP or combination of TAPs. The present disclosure also provides the use of cytotoxic T lymphocytes that specifically recognizes class I MHC molecules loaded with any of the TAP or combination of TAPs disclosed herein for generating an immune response against tumor cells expressing the human class I MHC molecules loaded with the TAP or combination thereof.

In an embodiment, the methods or uses described herein further comprise determining the HLA class I alleles expressed by the patient prior to the treatment/use, and administering or using TAPs that bind to one or more of the HLA class I alleles expressed by the patient. For example, if it is determined that the patient expresses HLA-A2*01 and HLA-B15*03, any combinations of (i) the TAPs of SEQ ID NO: SEQ ID NO:3, 6, 26, 30, 31, 39, 53, 55 and/or 58 (that bind to HLA-A2*01) and (ii) the TAPs of SEQ ID NO:2, 7, 11, 12, 15, 22, 29, 36, 38, 47, 48, and/or 59 (that bind to HLA-B15*03) may be administered or used in the patient.

In an embodiment, the TAP, nucleic acid, expression vector, T cell receptor, antibody/antibody fragment, cell (e.g., T lymphocyte, CAR T or NK cell, APC), and/or composition according to the present disclosure, or any combination thereof, may be used in combination with one or more additional active agents or therapies to treat cancer, such as chemotherapy (e.g., vinca alkaloids, agents that disrupt microtubule formation (such as colchicines and its derivatives), anti-angiogenic agents, therapeutic antibodies, EGFR targeting agents, tyrosine kinase targeting agent (such as tyrosine kinase inhibitors), transitional metal complexes, proteasome inhibitors, antimetabolites (such as nucleoside analogs), alkylating agents, platinum-based agents, anthracycline antibiotics, topoisomerase inhibitors, macrolides, retinoids (such as all-trans retinoic acids or a derivatives thereof), geldanamycin or a derivative thereof (such as 17-AAG), inhibitors of CDK4/6, TGF-β, WNT-β-catenin, MYC or PI3K, surgery, immune checkpoint inhibitors or immunotherapeutic agents (e.g., PD-1/PD-L1 inhibitors such as anti-PD-1/PD-L1 antibodies, CTLA-4 inhibitors such as anti-CTLA-4 antibodies, B7-1/B7-2 inhibitors such as anti-B7-1/B7-2 antibodies, TIM3 inhibitors such as anti-TIM3 antibodies, BTLA inhibitors such as anti-BTLA antibodies, CD47 inhibitors such as anti-CD47 antibodies, GITR inhibitors such as anti-GITR antibodies), antibodies against tumor antigens (e.g., anti-CD19, anti-CD22 antibodies), cell-based therapies (e.g., CAR T cells, CAR NK cells), and cytokines such as IL-2, IL-7, IL-21, and IL-15. In an embodiment, the TAP, nucleic acid, expression vector, T cell receptor, cell (e.g., T lymphocyte, APC), and/or composition according to the present disclosure is administered/used in combination with an immune checkpoint inhibitor. In an embodiment, the TAP, nucleic acid, expression vector, T cell receptor, cell (e.g., T lymphocyte, APC), and/or composition according to the present disclosure is administered/used in combination with inhibitors of CDK4/6, TGF-β and/or WNT-β-catenin. Several CDK4/6 inhibitors are in clinical trials including Palbociclib (PD-0332991, Ibrance), Ribociclib (LEE-011, Kisqali), Abemaciclib (LY2835219, Verzenios), SHR6390 and Trilaciclib (G1T28). Inhibitors of TGF-β include antisense inhibitors such as AP12009 (Trabedersen) and ISTH0036, antibodies and ligand traps such as GC1008 (Fresolimumab), LY2382770, and P144, vaccines targeting the TGF-β pathway such as Belagenpumatucel-L (Lucanix™), and FANG™ or vigil (Gemogenovatucel-T), as well as small molecule inhibitors such as LY2157299 (Galunisertib) and TEW-7197. Inhibitors of the WNT-β-catenin pathway include amino acid starvators (asparaginase), GSK3 inhibitors, C2 (

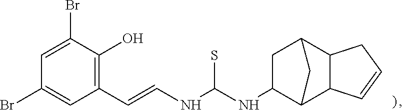 [SMILES: Oc1c(Br)cc(Br)cc1/C=C/NC(S)NC1CC2CC1C1C=CCC21]

WNT974, ETC-1922159, RXC004, CGX1321, OTSA101-DTPA-90Y, Vantictumab (OMP-18R5), Ipafricept (OMP-54F28), PRI-724, SM08502, secreted frizzled-related proteins/peptides and Tankyrase inhibitors (XAV939, JW-55, RK-287107, and G007-LK).

The additional therapy may be administered prior to, concurrent with, or after the administration of the TAP, nucleic acid, expression vector, T cell receptor, antibody/antibody fragment, cell (e.g., T lymphocyte, CAR T or NK cell, APC), and/or composition according to the present disclosure.

### EXAMPLES

The present disclosure is illustrated in further details by the following non-limiting examples.

### Example 1: Materials and Methods

Human iPSC Culture

hiPSC22 cells derived from male adult human skin fibroblasts using defective polycistronic retroviruses expressing OCT4, SOX2, KLF4, and c-MYC were obtained from Takara Bio (Cellartis human iPS cell line 22). hiPSC22 cells were cultured in the Cellartis® DEF-CS™ 500 Basal Medium with Additives (Takara Bio) on coated (Cellartis DEF-CS 500 COAT-1, Takara Bio) cell culture vessels according to the manufacturer's instructions. Fibro-iPSC.1 and Fibro-iPSC.2 cells are biological replicates of the same iPS cell line reprogrammed from female adult human dermal fibroblasts using lentiviruses expressing OCT4, SOX2, NANOG, and LIN28, as per (Hong et al., 2011), and were provided by Dr. Mick Bathia (McMaster University, Ontario, Canada). Fibro-iPSC.1 and Fibro-iPSC.2 were cultured on Matrigel® (Corning, diluted in DMEM/F-12 from Gibco)-coated cell culture vessels in mTeSR1 medium (STEMCELL), according to the manufacturer's instructions. All iPSCs were passaged using the Gentle Cell Dissociation Reagent (STEMCELL) or were dissociated to single cells using TrypLE Express (Gibco) and washed with DPBS (Gibco) for downstream analyses. After removing 3-5×10⁶ iPSCs for RNA-seq and 5×10⁶ cells for flow cytometry, iPSCs were pelleted and stored at −80 degrees C. until MS analysis. For IFN-γ-treated samples, iPSCs were treated with a final concentration of 40 ng/mL recombinant human IFN-γ (Gibco) for 72 hours before collection. MS analyses were performed on two fractions per iPS cell line as following, for each fraction: 250×10⁶ cells for untreated Fibro-iPSC.1 and Fibro-iPSC.2, 375×10⁶ cells for untreated hiPSC22, and 100-125×10⁶ cells for all IFN-γ-treated iPSC samples.

### Flow Cytometry Analysis

Single-cell suspensions were stained with PerCP-Cy5.5 Mouse anti-Oct3/4, PE Mouse anti-SSEA-1, Alexa Fluor 647 Mouse anti-SSEA-4 antibodies or the respective isotypes (Human and Mouse Pluripotent Stem Cell Analysis Kit, BD Biosciences), APC/Cyanine7 anti-human/mouse SSEA-3 (BioLegend) or the APC-Cy™7 Rat IgM, K Isotype Control (BD Biosciences) according to the manufacturers' instructions. Surface HLA-A,B,C molecules were quantified using a QIFIKIT (FITC conjugate, Agilent Dako) as per the manufacturer's instructions. Flow cytometry experiments were performed on a ZE5 (Bio-Rad), and data were analyzed using the FlowJo software.

### RNA Extraction and Sequencing

Total RNA extraction was done using TRIzol™ (Invitrogen) and further purification with the RNeasy Micro Kit (QIAGEN) from 3×10⁶ Fibro-iPSC.2 and Fibro-iPSC.2_IFN cells, and from 5×10⁶ cells for all other samples. The RNA quantification was performed using a QuBIT (Life Technologies), and the RNA quality was assessed using a Bioanalyzer Nano (Agilent), and all samples had an RNA integrity number of 10. cDNA library preparation was done using 1000 ng RNA for hiPSC22_IFN and 4000 ng RNA for all other samples, using the KAPA Hyperprep RNAseq stranded kit (KAPA) with polyA capture. 9 and 7 PCR cycles for hiPSC22_IFN and all other iPSC samples, respectively, were used for library amplification. Libraries were quantified by QuBit, and average library length was evaluated with the BioAnalyzer DNA1000. All libraries were diluted to 10 nM and normalized by qPCR using the KAPA library quantification kit (KAPA). Libraries were pooled to equimolar concentration. Sequencing was performed with the Illumina Nextseq500 using the Nextseq High Output 150 cycles (2×80 bp for hiPSC22 and hiPSC22_IFN, and 2×75 bp for all other iPSCs) using 2 pM of the pooled libraries. Around 180×10⁶ paired-end reads were generated per hiPSC22 sample (in three technical replicates pooled for MS database generation), 360×10⁶ paired-end reads for hiPSC22_IFN, and 230×10⁶ paired-end reads for all other iPSC samples. Library preparation and sequencing were done at the Institute for Research in Immunology and Cancer (IRIC) Genomics Platform.

### Database Generation for Shotgun Mass Spectrometry Analyses.

Generation of personalized canonical proteomes. This was conducted as previously described (Laumont et al., 2018). Briefly, RNA-seq reads were trimmed using Trimmomatic v0.35 and aligned to GRCh38.88 using STAR v2.5.1b (Dobin et al., 2013) running with default parameters except for --alignSJoverhangMin, --alignMatesGapMax, --alignIntronMax, and --alignSJstitchMismatchNmax parameters for which default values were replaced by 10, 200,000, 200,000 and “5-1 5 5”, respectively, to generate bam files. Single-base mutations with a minimum alternate count setting of 5 were identified using freeBayes v1.0.2-16-gd466dde (Garrison and Marth, 2012). Transcript expression was quantified in transcripts per million (tpm) with kallisto v0.43.0 with default parameters. Finally, we used pyGeno (Daouda et al., 2016) to insert high-quality sample-specific single-base mutations (freeBayes quality>20) in the reference exome and export sample-specific sequences of known proteins generated by expressed transcripts (tpm>0) to generate fasta files of personalized canonical proteomes.

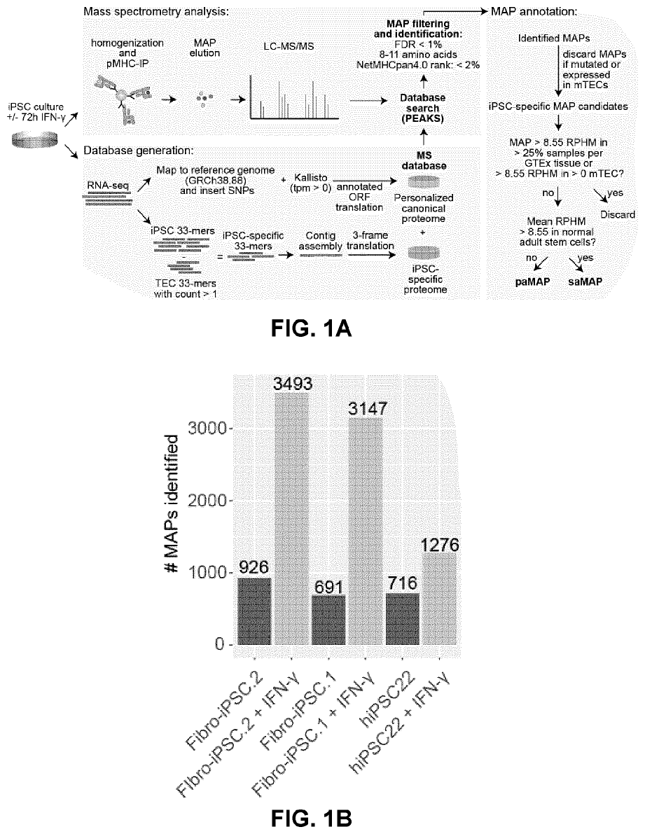

Generation of iPSC and mTEC k-mer databases. This was conducted as previously described (Laumont et al., 2018), with the following exceptions: 8 mTEC samples (GEO accessions GSE127825, GSE127826) were used instead of 6 mTECs, and the k-mer occurrence allowed in mTECs was 1 instead of 0 (see hereafter, FIG. 1A for schematic). Briefly, R1 and R2 fastq files of each sample were trimmed as reported above, and the reverse mapping reads (R1 for hiPSC22, and R2 for Fibro-iPSC.1 and Fibro-iPSC.2, with or without IFN-γ) were reverse complemented using the fastx_reverse_complement function of the FASTX-Toolkit v0.0.14. K-mer databases (24 or 33-long) were generated using Jellyfish v2.2.3 (Margais and Kingsford, 2011). A single k-mer database was generated for each iPSC sample, while the eight mTEC samples were combined in a unique database by concatenating their fastq files. Because the duration of k-mer assembly increases exponentially above 30 million k-mers, each iPSC 33-nucleotide-long k-mer database was filtered based on a sample-specific threshold on occurrence (the number of times that a given k-mer is present in the database) in order to reach a maximum of 30 million k-mers for the assembly step. After this filtering, k-mers present more than once in the mTECs k-mer database were removed from each sample database, and remaining k-mers were assembled into contigs with NEKTAR, an in-house developed software. Briefly, one of the submitted 33-nucleotide-long k-mer is randomly selected as a seed that is extended from both ends with consecutive k-mers overlapping by 32 nucleotides on the same strand (-r option disabled, as stranded sets of k-mers were used). The assembly process stops when either no k-mers can be assembled or when more than one k-mer fits (-a 1 option for linear assembly). Then a new seed is selected, and the assembly process resumes until all k-mers from the submitted list have been used once. Finally, the contigs were 3-frame translated using an in-house python script, amino acid sequences were split at internal stop codons and the resulting subsequences were concatenated with the respective personalized canonical proteome for each sample.

### Isolation of MHC-Associated Peptides

The W6/32 antibodies (BioXcell) were incubated in PBS for 60 minutes at room temperature with PureProteome protein A magnetic beads (Millipore) at a ratio of 1 mg of antibody per mL of slurry. Antibodies were covalently cross-linked to magnetic beads using dimethylpimelidate as described (Lamoliatte et al., 2017). The beads were stored at 4° C. in PBS pH 7.2. Frozen hiPSC22 pellets were thawed and resuspended in PBS pH 7.2 up to 1 mL and solubilized by adding 1 mL of detergent buffer containing PBS pH 7.2, 1% (w/v) CHAPS (Sigma) supplemented with Protease inhibitor cocktail (Sigma). Frozen Fibro-iPSC.1 and Fibro-iPSC.2 pellets were thawed and resuspended in PBS pH 7.2 up to 1 ml and solubilized by adding 1 mL of detergent buffer containing 0.5% (w/v) sodium deoxycholate (Thermo Fisher)/0.4 mM iodoacetamide (Sigma)/2% (w/v) Octyl β-D-glucopyranoside (Sigma)/2 mM EDTA (Promega) supplemented with Protease inhibitor cocktail (Sigma). Solubilized cell pellets were incubated for 60 minutes with tumbling at 4° C. and then spun at 16600×g for 20 minutes at 4° C. Supernatants were transferred into new tubes containing 1 mg of W6/32 antibody covalently-cross-linked protein A magnetic beads per sample and incubated with tumbling for 180 minutes at 4° C. Samples were placed on a magnet to recover bound MHC I complexes to magnetic beads. Magnetic beads were first washed with 8×1 mL PBS, then with 1×1 mL of 0.1×PBS, and finally with 1×1 mL of water. MHC I complexes were eluted from the magnetic beads by acidic treatment using 0.2% formic acid (FA). To remove any residual magnetic beads, eluates were transferred into 2.0 mL Costar mL Spin-X centrifuge tube filters (0.45 μm, Corning) and spun for 2 minutes at 855×g. Filtrates containing peptides were separated from MHC I subunits (HLA molecules and β-2 microglobulin) using homemade stage tips packed with two 1 mm diameter octadecyl (C-18) solid-phase extraction disks (EMPORE). Stage tips were pre-washed first with methanol, then with 80% acetonitrile (ACN) in 0.2% trifluoroacetic acid (TFA), and finally with 0.2% FA. Samples were loaded onto the stage tips and washed with 0.2% FA. Peptides were eluted with 30% ACN in 0.1% TFA, dried using vacuum centrifugation, and then stored at −20° C. until MS analysis.

### Mass Spectrometry Analyses

Dried peptide extracts were resuspended in 4% formic acid and loaded on a homemade C18 analytical column (15 cm×150 μm i.d. packed with C18 Jupiter Phenomenex) with a 56-min gradient (hiPSC22, hiPSC22_IFN) or 106-minute gradient (all other samples) from 0% to 30% acetonitrile (0.2% formic acid) and a 600 nL/min flow rate on an EasynLC II system. Samples were analyzed with a Q-Exactive HF mass spectrometer (Thermo Fisher Scientific) in positive ion mode with Nanospray 2 source at 1.6 kV. Each full MS spectrum, acquired with a 60,000 resolution was followed by 20 MS/MS spectra, where the most abundant multiply charged ions were selected for MS/MS sequencing with a resolution of 30,000, an automatic gain control target of 2×10⁴, an injection time of 100 ms (hiPSC22_IFN) or 800 ms (all other samples) and collisional energy of 25%.

### Bioinformatic Analyses

All analyses were conducted on trimmed data, and all alignments were made with STAR on the GRCh38.88 genome version as described in previous sections unless otherwise mentioned. Identification of MAPs. All liquid chromatography (LC)-MS/MS (LC-MS/MS) data were searched against the relevant database using PEAKS 10.5 (Bioinformatics Solution Inc.). For peptide identification, tolerance was set at 10 ppm and 0.01 Da for precursor and fragment ions, respectively. The occurrences of oxidation (M) and deamidation (NQ) were set as variable modifications. Following peptide identification, we used the modified target-decoy approach built in PEAKS to apply a sample-specific threshold on the PEAKS scores to ensure a false discovery rate (FDR) of 1%, calculated as the ratio between the number of decoy hits and the number of target hits above the score threshold. PEAKS scores corresponding to a 1% FDR for each sample were as following: 14 (hiPSC22), 15 (hiPSC22_IFN), 15 (Fibro-iPSC.1), 14 (Fibro-iPSC.1_IFN), 16 (Fibro-iPSC.2), 14 (Fibro-iPSC.2_IFN). Peptides that passed the threshold were further filtered to match the following criteria: peptide length between 8 and 11 amino acids, binding affinity rank to the sample's HLA alleles<2% based on NetMHCpan-4.0 (Jurtz et al., 2017) (FIG. 1A). These filtering steps were done with the use of MAPDP (Courcelles et al., 2020).

Identification of paMAPs. To identify paMAP candidates, each MAP and its coding sequence were queried in the relevant iPSC and mTEC canonical proteomes or the iPSC and mTEC 24-nucleotide-long k-mer databases, respectively, as previously described (Laumont et al., 2018). MAPs were retained as paMAP candidates if MAPs were not found in the mTEC canonical proteome, or if all possible MAP-coding sequences (MCS) for a given MAP i) were expressed below 2 KPHM (minimum occurrence of the MCS's 24-nucleotide-long k-mer set per hundred million reads) in mTECs, and ii) had a KPHM fold change superior or equal to 10 in iPSCs compared to mTECs.

Since leucine and isoleucine variants are not distinguishable by standard MS approaches, paMAP candidates for which an existing variant was flagged as a non-paMAP candidate were discarded unless they had a higher RNA expression than the variant. The genomic location of paMAP candidates was assigned by mapping reads containing their coding sequences on the reference genome using IGV (Robinson et al., 2011) and BLAT (tool from the UCSC genome browser). RepeatMasker (in the UCSC genome browser) was used to verify the overlap with EREs.

The RNA expression of paMAP candidates was evaluated in the RNA-seq of GTEx, mTECs, and adult stem cell (ASC) samples (FIG. 1A; see details in section RNA expression of MAPs below) as previously described (Ehx et al., 2021). paMAP candidates containing nucleotide variants in the MCS that did not correspond to known germline polymorphisms (dbSNP149) were classified as mutated MAPs and discarded from the analysis. All MAPs for which at least one MCS was successfully aligned to the reference genome were retained. paMAP candidates that passed the RNA expression filters in GTEx samples and ASCs (see MAP annotation in FIG. 1A) were considered paMAPs. paMAP candidates that passed the RNA expression filters in GTEx and mTEC samples but not in ASCs were considered saMAPs.

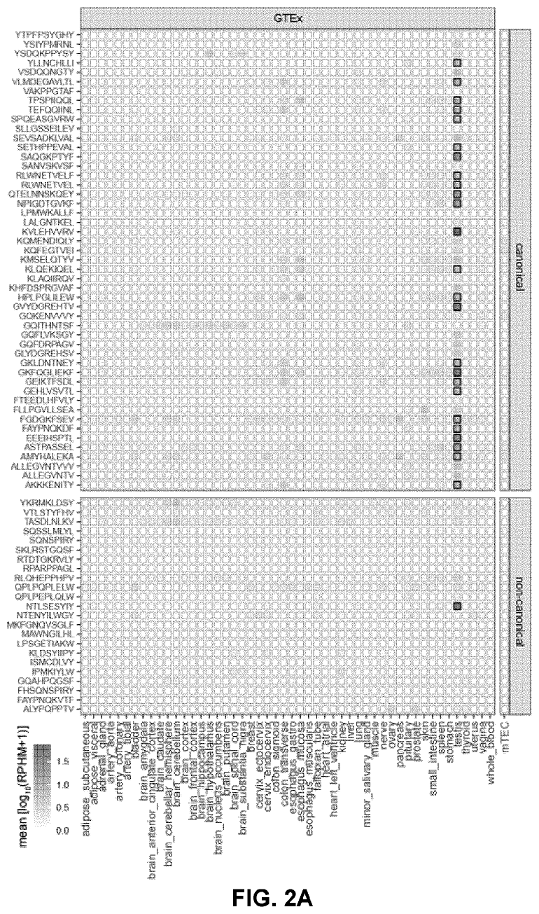

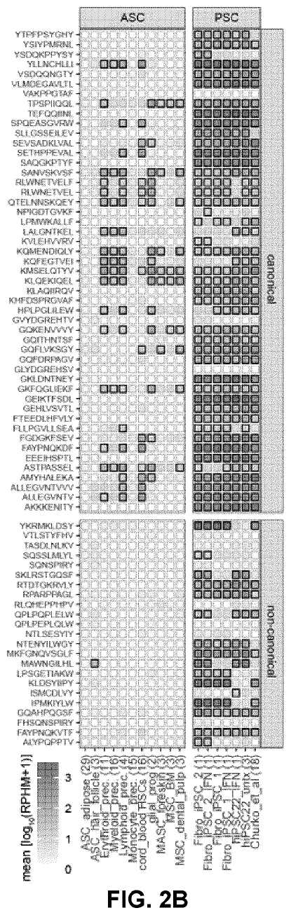

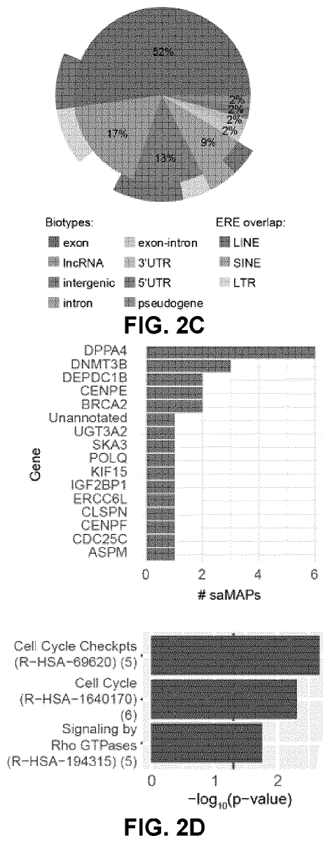

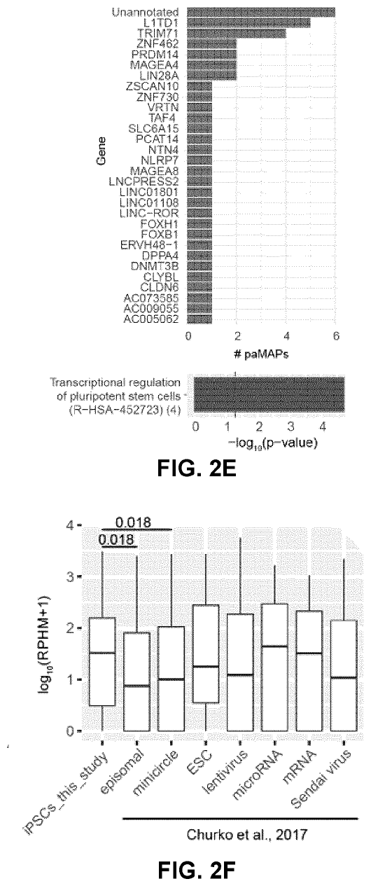

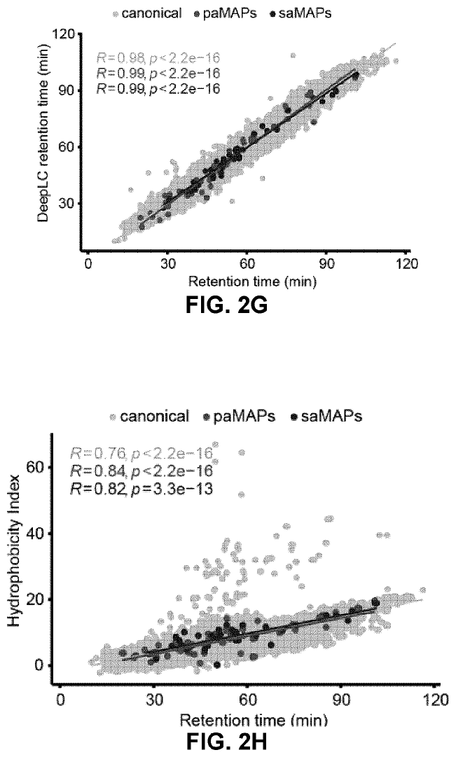

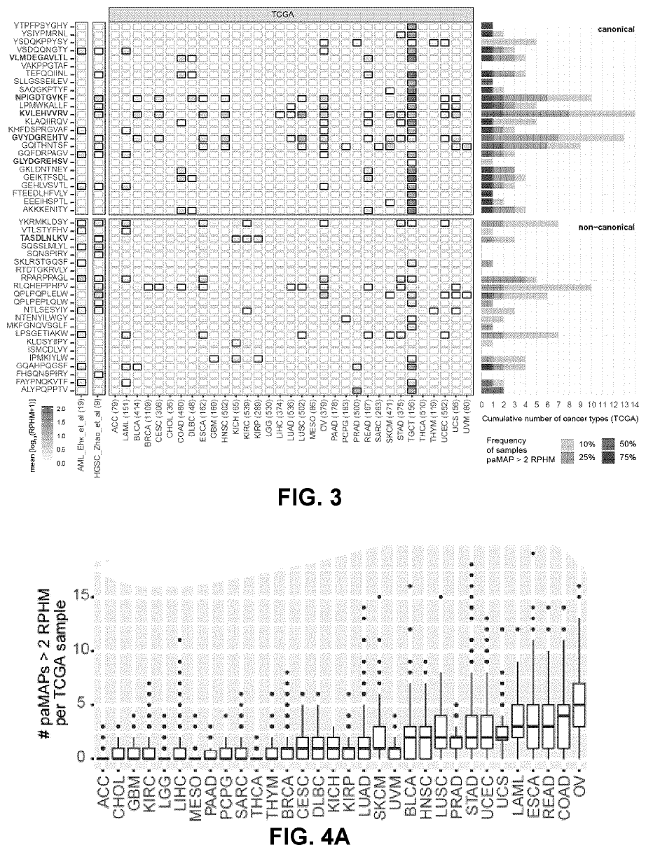

RNA expression of MAPs. The RNA expression of paMAP candidates was evaluated in RNA-seq samples (GTEx, PSCs, ASCs, TCGA; FIG. 1A, FIGS. 2A-B, FIG. 3) as previously described (Ehx et al., 2021). Briefly, all MAP amino acid sequences were reverse translated into all possible nucleotide sequences with an in-house python script (deposited to Zenodo at DOI: 3739257). Next, all these possible sequences were mapped to the genome with GSNAP (Wu and Nacu, 2010), with -n 1000000 option, to locate all genomic regions capable of coding for a given MAP. To confidently capture MAP coded by sequences overlapping splice sites, we also mapped the possible MCS's to the transcriptome (cDNA & non-coding RNA) to extract (samtools faidx with --length 80 option) large portions (80 nucleotides) of reference transcriptomic sequences that we then mapped on the reference genome (GSNAP, with --use-splicing and --novelsplicing=1 options). For all paMAP candidates, the genomic alignment of all reads containing their coding sequence was also performed. The outputs of GSNAP were filtered to only keep perfect matches between the sequences and the reference to generate a bed file containing all possible genomic regions susceptible to code for a given MAP. By using samtools view (—F256 option), grep and wc (—I option), the number of reads containing the MAP coding sequences at their respective genomic location was counted in each desired RNA-seq sample aligned to the reference genome with STAR (bam file). The BAM Slicing function from the GDC Data Portal (https://docs.gdc.cancer.gov/API/Users_Guide/BAM_Slicing/) was used to count the number of reads at each genomic location in the GRCh38 alignment files for TCGA samples. Finally, all read counts (from different regions and coding sequences) for a given MAP were summed and normalized to the total number of reads sequenced in each assessed sample to obtain a reads-per-hundred-million (RPHM) count.

Prediction of MAP retention time and hydrophobicity index. DeepLC 0.1.16 (Bouwmeester et al., 2020) was used to predict MAP retention times within MAPDP (Courcelles et al., 2020). SSRcalc (Krokhin, 2006) (http://hs2.proteome.ca/SSRCalc/SSRCalcQ.html) was used to calculate hydrophobicity indices based on peptide sequences.

Pathway enrichment analysis. paMAP- or saMAP-source genes (when annotated) were submitted to the “Statistical over-representation test” using Reactome pathways (version 65) as the annotation set in PANTHER v20200728 (Mi et al., 2021). The whole list of Homo sapiens genes was used as a reference. The statistical significance of each pathway's enrichment was assessed using Fisher's exact test, with the Bonferroni correction for multiple testing. Only pathways with a positive enrichment and an adjusted p-value<0.05 were kept.

Single-sample gene set enrichment analyses (ssGSEA). ssGSEA for paMAP- and saMAP-source genes, or for the stemness gene sets compiled by (Miranda et al., 2019), were performed using the GSVA package in R, without normalization, using TPM values quantified using kallisto (Bray et al., 2016) as described in previous sections. The resulting values were subsequently normalized by the absolute difference between the minimum and the maximum (min-max normalization) across gene sets and samples.

Sample clustering. Transcript expression quantifications performed with kallisto (Bray et al., 2016) with default parameters were converted into gene-level counts using the R package tximport. The edgeR package was then used to filter out lowly expressed genes and perform TMM normalization across the samples of interest. Normalized count per million (cpm) values were used to perform sample clustering based on the expression of the ESC-associated genes from Set 1 in (Ben-Porath et al., 2008). The heatmap.2 function was used to generate the expression heatmap and sample clustering using the default hclust function.

### TCGA Analyses

All tumor samples from TCGA were included unless otherwise specified. Testicular germ cell tumor (TGCT) samples were excluded from analyses performed across cancer types due to the presence of canonical paMAPs in the normal testis from GTEx. Mutation rate data were retrieved from Firebrowse (http://firebrowse.org/) as the number of nonsynonymous mutations per base (rate non column). Purity estimates for solid tumors were obtained from (Aran et al., 2015). Molecular subtype and tumor grade information were obtained using the TCGAbiolinks (Colaprico et al., 2016) package in R, while the curated clinical-stage data from (Liu et al., 2018).

Predicted paMAP and saMAPpresentation. The HLA alleles of each TCGA patient obtained using Polysolver (Castro et al., 2019) were kindly provided by Dr. Hannah Carter (UC San Diego). Promiscuous binders for a given MAP (all HLA alleles capable of presenting the MAP) were obtained using NetMHCpan-4.0 (Jurtz et al., 2017), and were those HLA alleles for which the given MAP had a binding affinity rank<2%. A given MAP (paMAP or saMAP) was considered as presented in a sample if it had an expression>0 RPHM and at least one of the patient's HLA allotypes was a potential binder. If the patient expressed more than one HLA allele capable of presenting a MAP, the MAP was counted as presented once.

Survival analysis. Pan-cancer curated clinical data for TCGA patients were obtained from (Liu et al., 2018). The cancer types for which the overall survival data were not recommended for use by (Liu et al., 2018) were excluded from the analysis. Only samples from primary solid tumors were kept, except for melanoma (SKCM) and AML, for which all samples with data available were used. The hazard ratio for the association between overall survival and the number of paMAPs (or saMAPs) expressed or with predicted presentation (see above) was conducted using the Cox proportional hazards model with the coxph function from the R package survival. For analyses using the number of paMAPs or saMAPs with predicted presentation (HLA-MAP), the Cox model controlled either for the number of paMAPs or saMAPs expressed per sample, since these two metrics are correlated and patients expressing more paMAPs and saMAPs are expected to have a worse prognosis.

Correlations and gene expression analyses. All analyses were performed in R. RNA-seq gene expression data for hg38 were retrieved as upper quartile-normalized fragments per kilobase of transcript per million mapped reads (FPKM-UQ) using the TCGAbiolinks package for each cancer type. The expression data were then merged across cancers. For genes with duplicate entries, we selected the one with the highest average expression across cancers. Merged FPKM-UQ values were then used to calculate ssGSEA scores for the hallmark gene sets from MSigDB (Liberzon et al., 2015) (http://www.gsea-msigdb.org/gsea/index.jsp), as described in the ssGSEA section above. Non-normalized ssGSEA scores were then used to perform Spearman's correlations with the number of paMAPs and saMAPs expressed per sample within cancer types, using the rcorr function. The Spearman correlations using the estimated purity from (Aran et al., 2015) as a covariate were performed using the pcor.test function from the ppcor package. P-values were adjusted using the Benjamini-Hochberg method.

For the correlation between the number of paMAPs and saMAPs per sample and gene expression, the RNA-seq expression data were retrieved as HTSeq-Counts using the TCGAbiolinks package for each cancer type. For genes with duplicate entries, we selected the one with the highest average expression across cancers. The edgeR package was then used to normalize counts using the TMM normalization after removing lowly expressed genes using the filterByExpr function (min. count of 10). Spearman correlations between the resulting normalized count per million (cpm) values and the number of paMAPs and saMAPs expressed per sample were performed using the rcorr function. Finally, the resulting p-values were corrected for multiple testing using the Benjamini-Hochberg method with the p.adjust function. Only samples from primary solid tumors were kept, except for melanoma (SKCM) and AML, for which all samples with data available were used.

HTSeq counts obtained as above were merged across cancer types for the differential gene expression analysis across cancers. For genes with duplicate entries, the one with the highest average expression across cancers was selected. The edgeR package was used to remove lowly expressed genes (genes with >1 cpm in >50 samples were kept) and perform TMM normalization. The limma package with the voom method was then used to assess differential gene expression between samples with high paMAP vs. high saMAP numbers, controlling for tumor purity and cancer types. Only samples with purity estimates from (Aran et al., 2015) were included. TGCT was also excluded. Genes with absolute fold change >2 and adjusted p-value<0.05 were considered differentially expressed.

Methylation and focal CNV analyses. Processed level 3 methylation data (HM27 for TCGA-OV and HM450 for all other cancer types) for TCGA samples were retrieved using the TCGAbiolinks package. Only probes within 2 kb of the transcription start site of a given paMAP- or saMAP-source gene were kept. Spearman correlations were then performed between the RPHM expression of each MAP of interest with the beta values for the respective gene within cancers. The mean beta value was used for genes associated with multiple probes. The correlation results for TCGA-OV using HM27 were merged with the HM450 results for all other cancers for plotting. The p-values were adjusted for multiple testing using the Benjamini-Hochberg method. Only HM450 beta values were used for correlations across cancer types without TGCT.

For focal CNV correlations, processed hg38 gene-level copy number scores were retrieved using the TCGAbiolinks package. DNA copy-number changes within paMAP or saMAP coding regions were used to perform Spearman correlations with the expression (RPHM) of each MAP of interest within cancers. Mean copy-number values were used for multiple segments associated with a MAP-coding region. TGCT samples were excluded from correlation analyses across cancers. p-values were adjusted for multiple testing using the Benjamini-Hochberg method.

Genomic correlations with paMAP and saMAP counts. TCGA Unified Ensemble “MC3” somatic mutation (SNP and INDEL) calls (Ellrott et al., 2018) were downloaded from the UCSC Xena Functional Genomics Explorer (Goldman et al., 2020) (https://xenabrowser.net/), where only gene-level non-silent mutation calls with filter=PASS were kept and converted to binary values (1, non-silent mutation; 0, WT). TCGA pan-cancer gene-level copy number variation (CNV) estimated using the GISTIC2 threshold method were downloaded from UCSC Xena, where estimated values were threshold converted to −2, −1, 0, 1, 2, representing homozygous deletion, single copy deletion, diploid normal copy, low-level copy number amplification, or high-level copy number amplification, respectively. Patients with more than one sample, those with TCGT, and those that did not have both somatic mutation and CNV data, were excluded from the analysis. The Chi-squared test was used to compare the number of patients expressing >0 paMAPs and saMAPs (>2 RPHM) vs. the others, among patients with WT or mutant variants of each gene. The same analysis was repeated for gene-level amplifications (1 and 2), and deletions (−2 and −1). Features were further selected for plotting based on their prevalence in paMAP- and saMAP-expressing samples, high statistical significance or association with signaling pathways of interest.

For FIG. 12D, the “MC3” somatic mutation (SNP and INDEL) calls downloaded from UCSC Xena were used. Patients with more than one sample were excluded from the analysis. We then used Fisher's exact test to compare the number of patients expressing≥median numbers of paMAPs and saMAPs (>2 RPHM) vs. the others among patients with WT or mutant variants of each gene. Comparisons with a p-value<0.05 were kept, and the top three genes with the most prevalent mutations in cancer samples expressing paMAPs and saMAPs above the median number per cancer type were plotted. Genes fulfilling these criteria in at least one cancer type were plotted in all cancer types if they had p-value<0.05 to emphasize common genomic events correlated with paMAP and saMAP expression across cancer types.

Immune infiltration analysis. xCell enrichment scores were calculated in R using the rawEnrichmentAnalysis function, which omits adjusting the raw scores (Aran et al., 2017b). Spearman correlations were performed between the raw cell type enrichment scores and the paMAP and saMAP counts per sample or the ssGSEA enrichment score for paMAP- and saMAP-source genes (using FPKM-UQ values as above), followed by p-value adjustment (Benjamini-Hochberg method) with the p.adjust function in R. Only primary solid tumor samples were used for correlations, except for SKCM and LAML.

### Immunogenicity Assays

Immunogenicity predictions. Immunogenicity predictions of paMAPs and saMAPs were performed using Repitope (Ogishi and Yotsuyanagi, 2019). Feature computation was performed with the predefined MHCI_Human_MinimumFeatureSet variable and the FeatureDF_MHCI and FragmentLibrary files provided on the Mendeley repository of the package (version Jul. 13, 2019; DOI: 10.17632/sydw5xnxpt.1).

In vitro peptide-specific T cell expansion. Peptide-specific CD8⁺ T cells from 4 healthy donors were expanded in vitro (D11, D12, D13, and D14). The expanded cells from D12 were used for FEST and tetramer staining assays, whereas the expanded cells from D11, D13, and D14 were used only for tetramer staining assays.

T cells were cultured as previously described, with minor modifications (Danilova et al., 2018). Briefly, on day 0, thawed PBMCs from each healthy donor (BioIVT) were T cell-enriched using the Human Pan T cell isolation kit (Miltenyi Biotec). T cells were resuspended at 2×10⁶/mL in AIM V media supplemented with 50 μg/mL gentamicin (ThermoFisher Scientific) and 1% HEPES. The T cell-negative fraction was irradiated at 30 Gy, washed, and resuspended at 2×10⁶/mL in AIM V media supplemented with 50 μg/mL gentamicin and 1% HEPES. 2.5 ml per well of both T cells and irradiated T cell-depleted cells were added to a 6-well plate, along with either a peptide alone, a peptide pool (up to 6 MAPs per pool, 1 μg/mL final concentration for each MAP) or without peptide. Cells were cultured for 10 days at 37° C., 5% CO₂. On day 3 and 7, half the culture media was replaced with fresh culture media containing 100 IU/mL IL-2, 50 ng/mL IL-7, and 50 ng/mL IL-15 (day 3) and 200 IU/mL IL-2, 50 ng/mL IL-7, and 50 ng/mL IL-15 (day 7). On day 10, thawed PBMCs from the same donor were used to generate a new batch of T cell-depleted cells. These cells were irradiated at 30 Gy and added to cultures at a 1:1 T cell:non-T cell ratio, along with 1 μg/mL of relevant peptide(s) or without peptide. On day 13 and 17, at least half the culture media was replaced with fresh culture media (final concentrations: 100 IU/mL IL-2, 25 ng/mL IL-7, and 25 ng/mL IL-15). On day 20, cells were harvested to perform tetramer staining and/or FEST assays.

FEST assays. For FEST assays, CD8⁺ cells were further isolated using the Human CD8⁺ T Cell Isolation Kit (Miltenyi Biotec). As a negative control, CD8⁺ T cells were also isolated from freshly thawed uncultured PBMCs of the same healthy donor. DNA was extracted from CD8⁺ T cells using a QIAGEN DNA blood mini kit (QIAGEN). TCR Vβ CDR3 sequencing was performed using the survey resolution of the immunoSEQ platform (Adaptive Biotechnologies). Raw data exported from the immunoSEQ portal were processed with the FEST web tool (www.stat-apps.onc.jhmi.edu/FEST).

Tetramer staining. Following 20 days coculture using peptide-loaded T cell-depleted cells and cytokines, 1×10⁶ cells were stained for 30 min at 4° C. with custom-made peptide-HLA tetramers (NIH) and then stained for 30 min at 4° C. with a CD8 monoclonal antibody (BD Biosciences). Cells were washed with PBS (containing 2% FBS) before acquisition with a Celesta cytometer (BD Biosciences). Data were analyzed using the FlowJo v10 Software (BD Biosciences).

Ex vivo peptide-specific T cell quantification. Frequencies of peptide-MHC-specific CD8 T cells without in vitro expansion were also determined for the four healthy donors (D11, D12, D13, and D14). 50×10⁶ to 180×10⁶ of thawed PBMCs were stained with 1 μg/mL of PE- and 5 μg/mL APC-labeled peptide-MHC tetramers (NIH Tetramer Core Facility) for 30 minutes at 4° C. After washing with ice-cold sorting buffer (PBS, 2 mM EDTA, 0.5% BSA), cells were resuspended in 450 μL ice-cold sorting buffer, and 50 μL of anti-PE and anti-APC antibody conjugated magnetic microbeads (Miltenyi Biotec), then incubated for 20 minutes at 4° C. Cells were then washed, and tetramer⁺ cells were magnetically enriched with LS columns (Miltenyi Biotec), following the manufacturer's instructions. The resulting tetramer⁺-enriched fractions were stained with APC-H7-conjugated anti-CD3, BB515-conjugated anti-CD8, BV510-conjugated anti-CD4, PerCP-Cy5.5-conjugated anti-CD14, CD16, CD19 antibodies (BD Biosciences) for 30 min at 4° C. and washed. The entire stained sample was then analyzed with 7-AAD on a FACS Celesta cytometer (BD Biosciences), and fluorescent counting beads (Thermo Fisher Scientific) were used to normalize the results. As a control, the antigen-specific CD8 T-cell repertoires targeting 3 HLA-A*02:01-restricted immunodominant epitopes: MelanA₂₇ (a melanoma-derived Ag, ELAGIGILTV, SEQ ID NO:143), NS3₁₀₇₃ (derived from hepatitis C virus, CINGVCWTV, SEQ ID NO:144), and Gag₇₇ (derived from human immunodeficiency virus, SLYNTVATL, SEQ ID NO:145) were also enriched.

### Quantification and Statistical Analysis

Unless mentioned in the figure legends, all statistical tests comparing two conditions were performed with the unpaired two-tailed Wilcoxon test in R. For multiple pairwise comparisons, the p-values were adjusted using the Benjamini-Hochberg method using the compare_means function in R. All box plots show the median and interquartile range (IQR), and whiskers extend to the largest value no further than 1.5*IQR from the box hinges. Unless mentioned, all correlations were performed using Spearman's correlation coefficient. Plots and statistical analyses were performed in R v3.6.5 or Python v3.6.7.

### Example 2: MS-Based Identification of paMAPs Using Human iPSCs

To identify paMAPs derived from all possible genomic regions, a proteogenomic strategy that was previously developed for TSA identification (Laumont et al., 2018) was used. In essence, iPS cell line-specific MS databases were constructed by combining 1) annotated proteome-derived sequences (canonical proteome) and 2) three-frame translations of non-canonical iPSC-specific contigs depleted of subsequences expressed in human medullary thymic epithelial cells (mTECs) (FIG. 1A). This method maintains an optimal database size and, due to the role of mTECs in mediating central tolerance, enables the identification of MAPs that may be immunogenic.

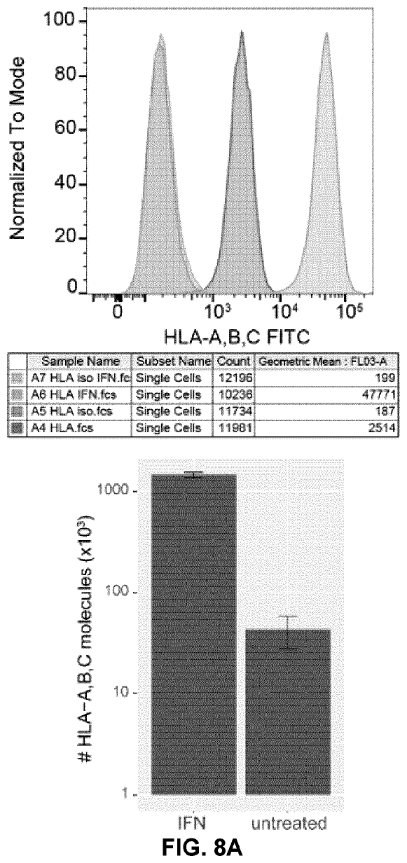

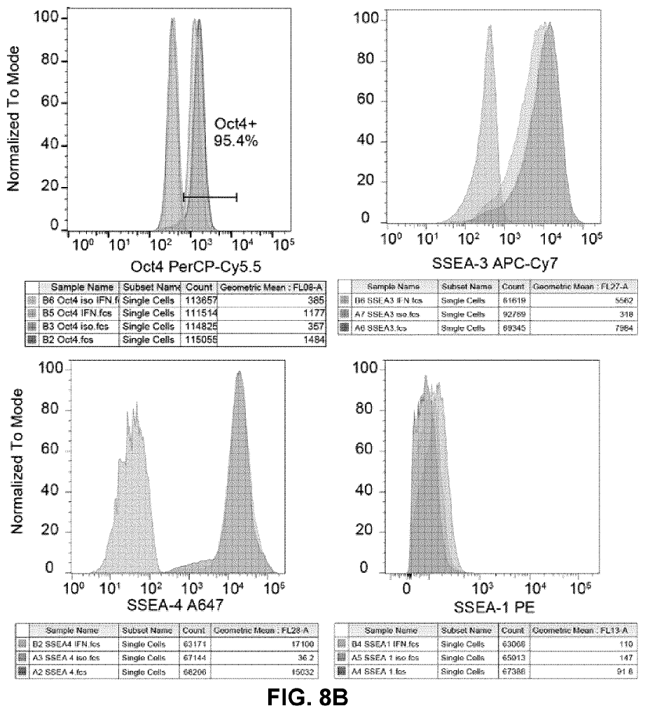

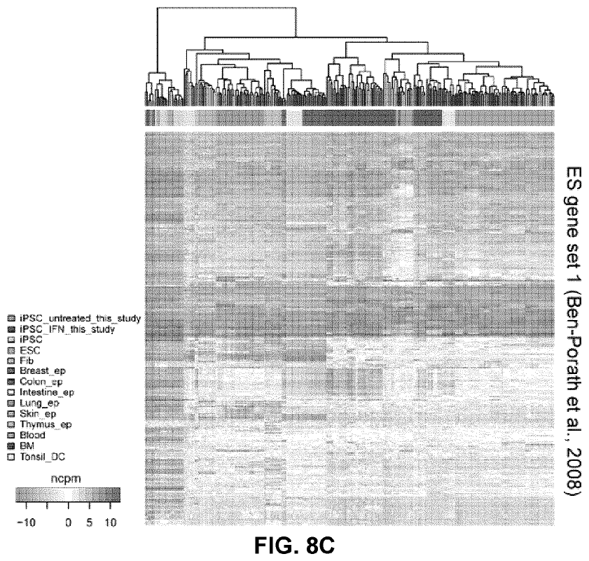

Because the abundance of MAPs is limiting in MS-based identifications and iPSCs express low levels of surface MHC I molecules (Suárez-Álvarez et al., 2010; Vogel and Marcotte, 2009), a 72 h-treatment with interferon-γ (IFN-γ) was performed prior to collection for MS analysis (FIG. 1A). IFN-γ treatment induced, on average, a 34-fold increase in surface HLA-A/B/C levels for the three fibroblast-derived iPSC samples studied (see Example 1), without affecting the expression of canonical pluripotency markers (Stewart et al., 2006) (FIG. 8A-C). As a result, this treatment allowed the detection of 1.8-4.5-fold more unique MAPs than for untreated iPSCs (FIG. 1B), thus expanding our search space for paMAPs.

### Example 3: The Immunopeptidome of iPSCs Reflects their Pluripotency State

The probability that a MAP will be presented at the cell surface depends mainly on two factors. First, on the expression of MHC-1 genes, which is high in hematopoietic cells and mTECs (MHC-1^{hi}) but low on non-inflamed extrathymic nonhematopoietic cells (MHC-1^{lo}) (Benhammadi et al., 2020). Second, on the expression of the MAP-coding sequence (MCS) (Bassani-Sternberg et al., 2015; Ehx et al., 2021; Pearson et al., 2016; Ruiz Cuevas et al., 2021). Indeed, an MCS expression inferior to 8.55 RPHM (reads per hundred million) corresponds to a probability of MAP generation lower than 5% in myeloid cells (Ehx et al., 2021). It may be assumed that the probability would even lower in extrathymic nonhematopoietic cells because they are MHC-1^{lo}. Hence, in the search for paMAPs, MAPs whose MCS were expressed at less than 8.55 RPHM (reads per hundred million) in 29 different healthy tissues from the GTEx dataset were selected (Genotype-Tissue Expression, (Lonsdale et al., 2013)). MCS expression in the testis was not an exclusion criterion because cells of the spermatocyte lineage do not express MHC-1 genes (Zhao et al., 2014) and may retain expression of some pluripotency markers (Izadyar et al., 2011; Wang et al., 2007; Zheng et al., 2009). Of the 5424 unique MAPs identified from untreated and IFN-γ-treated iPSCs, 72 (1.33%) matched the stringent expression profile (FIGS. 1A and 2A, Tables 1A-D). To distinguish MAPs associated with a stemness program as opposed to a pluripotency program, the 72 MAP-coding sequences in the RNA-seq of primary adult stem and progenitor cells (ASCs) from different origins were quantified: mesenchymal stem cells, bone marrow progenitors, hematopoietic stem cells from cord blood samples, glial progenitors. It was found that 26 MAPs were expressed in at least one ASC dataset and were termed stemness-associated MAPs (saMAPs, Tables 1C-D), whereas the remaining 46 MAPs were considered pluripotency-associated (paMAPs) (FIG. 2B, Tables 1A-B). Because single nucleotide variation (SNV) information for the somatic cells used for iPSC generation were lacking and it was not possible to discriminate between germline and reprogramming-associated mutations (Merkle et al., 2017), MAPs deriving from mutated DNA sequences were excluded from these analyses.

TABLE 1A
Characteristics of paMAPs identified herein

| Peptide sequence | | Gene | | Predicted | Immunogenicity |
|---|---|---|---|---|---|
| (SEQ ID NO:) | Source | Symbol | Ensembl GenelD | HLA allele | score |
| NTENYILWGY | lncRNA | AC005062 | ENSG00000243004 | HLA-A*01:01 | 0.26304 |
| (1) | | | | | |
| MKFGNQVSGLF | lncRNA | AC009055 | ENSG00000260834 | HLA-B*15:03 | 0.29712 |
| (2) | | | | | |
| RLQHEPPHPV | pseudogene | AC073585 | ENSG00000255624 | HLA-A*02:01 | 0.38736 |
| (3) | | | | | |
| LPMWKALLF (4) | annotated | CLDN6 | ENSG00000184697 | HLA-B*53:01 | 0.37296 |
| | exon | | | | |
| RPARPPAGL (5) | 5′UTR | DNMT3B | ENSG00000088305 | HLA-B*07:02 | 0.47264 |
| FAYPNQKVTF | exon-intron | DPPA4 | ENSG00000121570 | HLA-C*03:04, | 0.17104 |
| (6) | | | | HLA-C*02:10 | |
| GQAHPQGSF | lncRNA | ERVH48-1 | ENSG00000233056 | HLA-B*15:03 | 0.30584 |
| (7) | | | | | |
| YSDQKPPYSY | annotated | FOXB1 | ENSG00000171956 | HLA-A*01:01 | 0.13264 |
| (8) | exon | | | | |
| KLAQIIRQV (9) | annotated | FOXH1 | ENSG00000160973 | HLA-A*02:01 | 0.31952 |
| | exon | | | | |
| GEIKTFSDL (10) | annotated | L1TD1 | ENSG00000240563 | HLA-B*40:01 | 0.1748 |
| | exon | | | | |
| GKLDNTNEY | annotated | L1TD1 | ENSG00000240563 | HLA-B*15:03 | 0.19088 |
| (11) | exon | | | | |
| AKKKENITY (12) | annotated | L1TD1 | ENSG00000240563 | HLA-B*15:03 | 0.24296 |
| | exon | | | | |
| SAQGKPTYF | annotated | LIN28A | ENSG00000131914 | HLA-C*04:01 | 0.19016 |
| (13) | exon | | | | |
| EEEIHSPTL (14) | annotated | LIN28A | ENSG00000131914 | HLA-B*40:01 | 0.27 |
| | exon | | | | |
| SKLRSTGQSF | lncRNA | LINC01108 | ENSG00000226673 | HLA-B*15:03 | 0.20112 |
| (15) | | | | | |
| NTLSESYIY (16) | lncRNA | LINC01801 | ENSG00000267767 | HLA-A*01:01 | 0.23104 |
| QPLPQPLELW | lncRNA | LINC-ROR | ENSG00000258609 | HLA-B*53:01 | 0.55088 |
| (17) | | | | | |
| RTDTGKRVLY | lncRNA | LNCPRESS2 | ENSG00000249152 | HLA-A*01:01 | 0.19088 |
| (18) | | | | | |
| LPSGETIAKW | intergenic | NA | NA | HLA-B*53:01 | 0.28952 |
| (19) | | | | | |
| KLDSYIIPY (20) | intergenic | NA | NA | HLA-A*01:01 | 0.21496 |
| ISMCDLVY (21) | intergenic | NA | NA | HLA-A*01:01 | 0.362 |
| YKRMKLDSY | intergenic | NA | NA | HLA-B*15:03 | 0.18624 |
| (22) | | | | | |
| QPLPEPLQLW | intergenic | NA | NA | HLA-B*53:01 | 0.54832 |
| (23) | | | | | |
| IPMKIYLW (24) | intergenic | NA | NA | HLA-B*53:01 | 0.39112 |
| SQSSLMLYL | intronic | NTN4 | ENSG00000074527 | HLA-B*40:01 | 0.29256 |
| (25) | (antisense) | | | | |
| ALYPQPPTV | lncRNA | PCAT 14 | ENSG00000280623 | HLA-A*02:01 | 0.56536 |
| (26) | | | | | |
| YTPFPSYGHY | annotated | PRDM14 | ENSG00000147596 | HLA-A*01:01 | 0.37456 |
| (27) | exon | | | | |
| FTEEDLHFVLY | annotated | PRDM14 | ENSG00000147596 | HLA-A*01:01 | 0.1776 |
| (28) | exon | | | | |
| GQITHNTSF | annotated | SLC6A15 | ENSG00000072041 | HLA-B*15:03 | 0.2984 |
| (29) | exon | | | | |
| VTLSTYFHV | intronic | TAF4 | ENSG00000130699 | HLA-A*02:01 | 0.26056 |
| (30) | | | | | |
| GQFDRPAGV | annotated | TRIM71 | ENSG00000206557 | HLA-A*02:01 | 0.392 |
| (31) | exon | | | | |
| VSDQQNGTY | annotated | TRIM71 | ENSG00000206557 | HLA-A*01:01 | 0.21936 |
| (32) | exon | | | | |
| KHFDSPRGVAF | annotated | TRIM71 | ENSG00000206557 | HLA-C*04:01 | 0.2888 |
| (33) | exon | | | | |
| GEHLVSVTL | annotated | TRIM71 | ENSG00000206557 | HLA-B*40:01 | 0.30552 |
| (34) | exon | | | | |
| YSIYPMRNL | annotated | VRTN | ENSG00000133980 | HLA-C*03:04 | 0.26776 |
| (35) | exon | | | | |
| SQNSPIRY (36) | intronic | ZNF462 | ENSG00000148143 | HLA-B*15:03 | 0.2544 |
| FHSQNSPIRY | intronic | ZNF462 | ENSG00000148143 | HLA-A*01:01 | 0.24584 |
| (37) | | | | | |
| VAKPPGTAF | annotated | ZNF730 | ENSG00000183850 | HLA-B*15:03 | 0.3736 |
| (38) | exon | | | | |
| SLLGSSEILEV | annotated | ZSCAN10 | ENSG00000130182 | HLA-A*02:01 | 0.29272 |
| (39) | exon | | | | |
| TASDLNLKV | 3′UTR | CLYBL | ENSG00000125246 | HLA-C*03:04 | 0.266 |
| (40) | | | | | |
| VLMDEGAVLTL | annotated | L1TD1 | ENSG00000240563 | HLA-A*02:01 | 0.42496 |
| (41) | exon | | | | |
| TEFQQIINL (42) | annotated | L1TD1 | ENSG00000240563 | HLA-B*40:01 | 0.20448 |
| | exon | | | | |
| KVLEHVVRV | annotated | MAGEA4 | ENSG00000147381 | HLA-A*02:01 | 0.2992 |
| (43) | exon | | | | |
| GVYDGREHTV | annotated | MAGEA4 | ENSG00000147381 | HLA-C*04:01 | 0.86328 |
| (44) | exon | | | | |
| GLYDGREHSV | annotated | MAGEA8 | ENSG00000156009 | HLA-A*02:01 | 0.40472 |
| (45) | exon | | | | |
| NPIGDTGVKF | annotated | NLRP7 | ENSG00000167634 | HLA-B*53:01 | 0.30048 |
| (46) | exon | | | | |

TABLE 1B
Characteristics of paMAPs identified herein (continued)

| | | | | Number of | |
|---|---|---|---|---|---|
| | | | | cancer types | |
| | | | | with > = 10% | |
| Peptide sequence | | ERE | ERE | samples above | Previously |
| (SEQ ID NO:) | ERE | family | class | 2 RPHM | reported ? |
| NTENYILWGY | NA | NA | NA | 2 | No |
| (1) | | | | | |
| MKFGNQVSGLF | HERVH-int, | ERV1 | LTR | 1 | No |
| (2) | partial | | | | |
| | overlap | | | | |
| RLQHEPPHPV | NA | NA | NA | 10 | No |
| (3) | | | | | |
| LPMWKALLF (4) | NA | NA | NA | 5 | No |
| RPARPPAGL (5) | NA | NA | NA | 5 | No |
| FAYPNQKVTF | NA | NA | NA | 2 | No |
| (6) | | | | | |
| GQAHPQGSF | HERVH48- | ERV1 | LTR | 4 | No |
| (7) | int | | | | |
| YSDQKPPYSY | NA | NA | NA | 5 | No |
| (8) | | | | | |
| KLAQIIRQV (9) | NA | NA | NA | 5 | No |
| GEIKTFSDL (10) | L1MEd | L1 | LINE | 4 | No |
| GKLDNTNEY | L1TD1 | L1 | LINE | 3 | No |
| (11) | | | | | |
| AKKKENITY (12) | L1MEd | L1 | LINE | 4 | No |
| SAQGKPTYF | NA | NA | NA | 2 | No |
| (13) | | | | | |
| EEEIHSPTL (14) | NA | NA | NA | 2 | No |
| SKLRSTGQSF | NA | NA | NA | 1 | No |
| (15) | | | | | |
| NTLSESYIY (16) | L1ME3B | L1 | LINE | 3 | No |
| QPLPQPLELW | HERVH-int | ERV1 | LTR | 6 | No |
| (17) | | | | | |
| RTDTGKRVLY | NA | NA | NA | 0 | No |
| (18) | | | | | |
| LPSGETIAKW | HERV9NC- | ERV1 | LTR | 7 | No |
| (19) | int | | | | |
| KLDSYIIPY (20) | L1M3 | L1 | LINE | 1 | No |
| ISMCDLVY (21) | L1ME1 | L1 | LINE | 0 | No |
| | antisense | | | | |
| YKRMKLDSY | L1M3 | L1 | LINE | 7 | No |
| (22) | | | | | |
| QPLPEPLQLW | HERVH-int | ERV1 | LTR | 1 | No |
| (23) | | | | | |
| IPMKIYLW (24) | L1M3 | L1 | LINE | 4 | No |
| SQSSLMLYL | L1MEc | L1 | LINE | 0 | No |
| (25) | | | | | |
| ALYPQPPTV | HERVK-int | ERVK | LTR | 2 | No |
| (26) | | | | | |
| YTPFPSYGHY | NA | NA | NA | 1 | No |
| (27) | | | | | |
| FTEEDLHFVLY | NA | NA | NA | 1 | No |
| (28) | | | | | |
| GQITHNTSF | NA | NA | NA | 9 | No |
| (29) | | | | | |
| VTLSTYFHV | L1MA4A | L1 | LINE | 1 | No |
| (30) | | | | | |
| GQFDRPAGV | NA | NA | NA | 3 | No |
| (31) | | | | | |
| VSDQQNGTY | NA | NA | NA | 3 | No |
| (32) | | | | | |
| KHFDSPRGVAF | NA | NA | NA | 3 | No |
| (33) | | | | | |
| GEHLVSVTL | NA | NA | NA | 3 | No |
| (34) | | | | | |
| YSIYPMRNL | NA | NA | NA | 2 | No |
| (35) | | | | | |
| SQNSPIRY (36) | MIR, | MIR | SINE | 0 | No |
| | antisense | | | | |
| FHSQNSPIRY | MIR, | MIR | SINE | 0 | No |
| (37) | antisense | | | | |
| VAKPPGTAF | NA | NA | NA | 0 | No |
| (38) | | | | | |
| SLLGSSEILEV | NA | NA | NA | 1 | No |
| (39) | | | | | |
| TASDLNLKV | NA | NA | NA | 3 | PMID: 32047025 |
| (40) | | | | | |
| VLMDEGAVLTL | L1MEd | L1 | LINE | 4 | US2016/0279214 |
| (41) | | | | | |
| TEFQQIINL (42) | NA | NA | NA | | WO2020/058285 |
| KVLEHVVRV | NA | NA | NA | 14 | PMID: 21350607 |
| (43) | | | | | |
| GVYDGREHTV | NA | NA | NA | 13 | PMID: 11502003, |
| (44) | | | | | PMID: 10540345 |
| GLYDGREHSV | NA | NA | NA | 1 | PMID: 10352307 |
| (45) | | | | | |
| NPIGDTGVKF | NA | NA | NA | 10 | WO2018/138257 |
| (46) | | | | | |

TABLE 1C
Characteristics of saMAPs identified herein

| Peptide sequence | | Gene | | Predicted | Immunogenicity |
|---|---|---|---|---|---|
| (SEQ ID NO:) | Source | Symbol | Ensembl GeneID | HLA allele | score |
| GKFQGLIEKF | annotated | CDC25C | ENSG00000158402 | HLA-B*15:03 | 0.8116 |
| (47) | exon | | | | |
| KQMENDIQLY | annotated | CENPE | ENSG00000138778 | HLA-B*15:03 | 0.0847 |
| (48) | exon | | | | |
| ASTPASSEL | annotated | CLSPN | ENSG00000092853 | HLA-C*03:04 | 0.2182 |
| (49) | exon | | | | |
| RLWNETVELF | annotated | DEPDC1B | ENSG00000035499 | HLA-C*04:01 | 0.911 |
| (50) | exon | | | | |
| FGDGKFSEV | annotated | DNMT3B | ENSG00000088305 | HLA-C*03:04 | 1.2864 |
| (51) | exon | | | | |
| SEVSADKLVAL | annotated | DNMT3B | ENSG00000088305 | HLA-B*40:01 | 0.1455 |
| (52) | exon | | | | |
| AMYHALEKA | annotated | DNMT3B | ENSG00000088305 | HLA-A*02:01 | 0.5 |
| (53) | exon | | | | |
| FAYPNQKDF | annotated | DPPA4 | ENSG00000121570 | HLA-C*03:04, | 0.0308, 0.0919 |
| (54) | exon | | | HLA-C*02:10 | |
| ALLEGVNTVVV | annotated | DPPA4 | ENSG00000121570 | HLA-A*02:01 | 0.3886 |
| (55) | exon | | | | |
| SETHPPEVAL | annotated | DPPA4 | ENSG00000121570 | HLA-B*40:01 | 0.1729 |
| (56) | exon | | | | |
| SPQEASGVRW | annotated | DPPA4 | ENSG00000121570 | HLA-B*53:01 | 0.2415 |
| (57) | exon | | | | |
| YLLNCHLLI (58) | annotated | DPPA4 | ENSG00000121570 | HLA-A*02:01 | 0.0553 |
| | exon | | | | |
| GQFLVKSGY | annotated | IGF2BP1 | ENSG00000159217 | HLA-B*15:03 | 0.1989 |
| (59) | exon | | | | |
| QTELNNSKQEY | annotated | KIF15 | ENSG00000163808 | HLA-A*01:01 | 0.1568 |
| (60) | exon | | | | |
| MAWNGILHL | intergenic | NA | NA | HLA-C*02:10 | 0.0935 |
| (61) | | | | | |
| HPLPGLILEW | annotated | POLQ | ENSG00000051341 | HLA-B*53:01 | 0.014 |
| (62) | exon | | | | |
| TPSPIIQQL (63) | annotated | SKA3 | ENSG00000165480 | HLA-B*53:01 | 0.3482 |
| | exon | | | | |
| FLLPGVLLSEA | annotated | UGT3A2 | ENSG00000168671 | HLA-A*02:01 | 0.1943 |
| (64) | exon | | | | |
| SANVSKVSF | annotated | ASPM | ENSG00000066279 | HLA-B*15:03 | 0.9036 |
| (65) | exon | | | | |
| LALGNTKEL | annotated | BRCA2 | ENSG00000139618 | HLA-C*03:04 | 0.0397 |
| (66) | exon | | | | |
| KQFEGTVEI | annotated | BRCA2 | ENSG00000139618 | HLA-A*02:01 | 0.1429 |
| (67) | exon | | | | |
| KLQEKIQEL (68) | annotated | CENPE | ENSG00000138778 | HLA-A*02:01 | 0.1966 |
| | exon | | | | |
| KMSELQTYV | annotated | CENPF | ENSG00000117724 | HLA-A*02:01 | 0.0155 |
| (69) | exon | | | | |
| RLWNETVEL | annotated | DEPDC1B | ENSG00000035499 | HLA-A*02:01 | 0.1007 |
| (70) | exon | | | | |
| ALLEGVNTV | annotated | DPPA4 | ENSG00000121570 | HLA-A*02:01 | 0.0093 |
| (71) | exon | | | | |
| GQKENVVVY | annotated | ERCC6L | ENSG00000186871 | HLA-B*15:03 | 0.09 |
| (72) | exon | | | | |

TABLE 1D
Characteristics of saMAPs identified herein (continued)

| | | | | Number of cancer | |
|---|---|---|---|---|---|
| | | | | types with | |
| Peptide sequence | | ERE | ERE | > = 10% samples | Previously |
| (SEQ ID NO:) | ERE | family | class | above 2 RPHM | reported ? |
| GKFQGLIEKF | NA | NA | NA | 29 | No |
| (47) | | | | | |
| KQMENDIQLY | NA | NA | NA | 22 | No |
| (48) | | | | | |
| ASTPASSEL | NA | NA | NA | 25 | No |
| (49) | | | | | |
| RLWNETVELF | NA | NA | NA | 31 | No |
| (50) | | | | | |
| FGDGKFSEV | NA | NA | NA | 28 | No |
| (51) | | | | | |
| SEVSADKLVAL | NA | NA | NA | 22 | No |
| (52) | | | | | |
| AMYHALEKA | NA | NA | NA | 24 | No |
| (53) | | | | | |
| FAYPNQKDF | NA | NA | NA | 3 | No |
| (54) | | | | | |
| ALLEGVNTVVV | NA | NA | NA | 3 | No |
| (55) | | | | | |
| SETHPPEVAL | NA | NA | NA | 3 | No |
| (56) | | | | | |
| SPQEASGVRW | NA | NA | NA | 3 | No |
| (57) | | | | | |
| YLLNCHLLI (58) | NA | NA | NA | 3 | No |
| GQFLVKSGY | NA | NA | NA | 12 | No |
| (59) | | | | | |
| QTELNNSKQEY | NA | NA | NA | 23 | No |
| (60) | | | | | |
| MAWNGILHL | NA | NA | NA | 1 | No |
| (61) | | | | | |
| HPLPGLILEW | NA | NA | NA | 25 | No |
| (62) | | | | | |
| TPSPIIQQL (63) | NA | NA | NA | 30 | WO2003/050255 |
| FLLPGVLLSEA | NA | NA | NA | 5 | US10596241 |
| (64) | | | | | |
| SANVSKVSF | NA | NA | NA | 18 | WO2017/089781 |
| (65) | | | | | |
| LALGNTKEL | NA | NA | NA | 8 | Creative Biolabs |
| (66) | | | | | PEP-123419CQ |
| KQFEGTVEI | NA | NA | NA | 17 | PMID: 27841757 |
| (67) | | | | | |
| KLQEKIQEL | NA | NA | NA | 28 | PMID: 27841757 |
| (68) | | | | | |
| KMSELQTYV | NA | NA | NA | 32 | US2016/0280759 |
| (69) | | | | | |
| RLWNETVEL | NA | NA | NA | 32 | US2017/0037092 |
| (70) | | | | | |
| ALLEGVNTV | NA | NA | NA | 3 | WO2017/001491 |
| (71) | | | | | |
| GQKENVVVY | NA | NA | NA | 23 | US7250498 |
| (72) | | | | | |

It is notable that all but one saMAP were derived from annotated protein-coding exons, whereas 48% of the paMAPs were derived from allegedly non-coding genomic regions, in particular from annotated long non-coding RNAs (lncRNAs) (17%), intergenic (13%) and intronic (9%) sequences (FIGS. 2B-C, Tables 1A-D). Remarkably, nearly all paMAP-coding sequences from these three ostensibly non-coding regions had overlapping EREs comprising primarily long-interspersed nuclear element (LINE) and long terminal repeat (LTR) sequences (FIG. 2C and Table 1B). These elements, most notably LINE-1 (L1) and human endogenous retrovirus subfamily H (HERV-H), are derepressed during reprogramming and essential for the maintenance of pluripotency because they enhance the specific expression of lncRNAs and neighboring genes (Fort et al., 2014; Friedli et al., 2014; Kelley and Rinn, 2012; Klawitter et al., 2016; Lu et al., 2014). The major overlap with EREs provides a mechanistic rationale for the PSC-specific transcription and translation of these allegedly non-coding regions. By contrast, ERE overlap with canonical paMAP-coding sequences was found only in paMAPs derived from LINE1 type transposase domain containing 1 (L1TD1), a domesticated RNA-binding protein derived from an L1 retroelement and required for the self-renewal of PSCs (McLaughlin et al., 2014; Narva et al., 2012) (Table 1B).

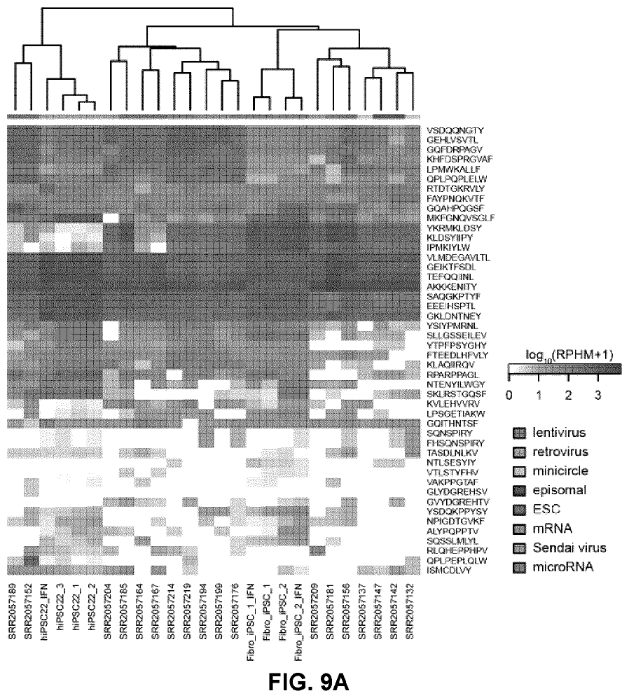

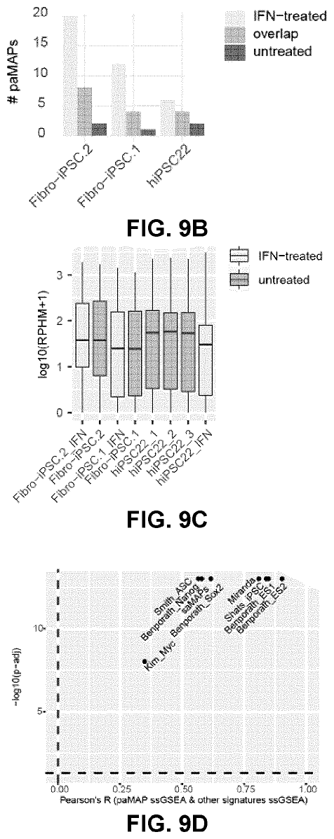

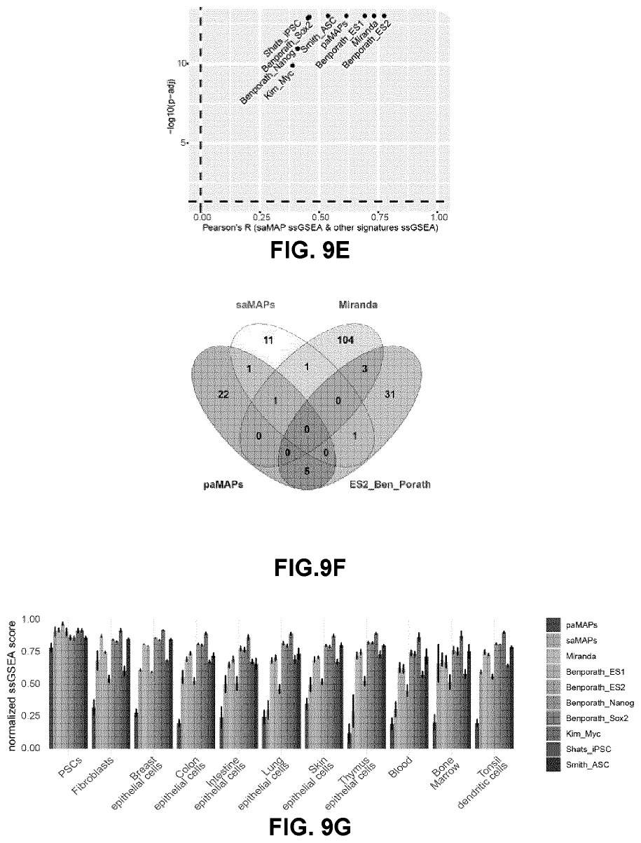

Several features of paMAPs reinforce their association to pluripotency. Firstly, the paMAP- and saMAP-source genes were non-redundant, except for two genes, DNMT3B and DPPA4. These two genes generated iPSC-specific expression of non-canonical MAPs, whereas their exonic MAPs were also highly expressed in ASCs (FIGS. 2B, D, E, and Tables 1A-D). Accordingly, the only biological pathway significantly enriched in the paMAP-source genes was transcriptional regulation of PSCs, represented by the pluripotency-regulating genes LIN28A, ZSCAN10, PRDM14, and DPPA4 (Chia et al., 2010; Hernandez et al., 2018; Wang et al., 2007; Zhang et al., 2016) (FIG. 2D). By contrast, saMAP-source genes were primarily involved in cell cycle regulation (FIG. 2E). Secondly, paMAPs were expressed similarly between ESCs and iPSCs generated from six different reprogramming methods (Churko et al., 2017) (FIG. 2F, 9A). Besides, paMAPs were expressed similarly in the untreated and IFN-γ-treated samples studied (FIGS. 9B and C). Finally, it was assessed whether annotated saMAP- and paMAP-source genes (Tables 1A-D) can infer stemness and pluripotency using single-sample gene set enrichment analysis (ssGSEA), as previously described (Barbie et al., 2009; Hanzelmann et al., 2013). To this end, the stemness signatures extracted by Miranda and colleagues from different cancer datasets was used (Miranda et al., 2019). The saMAP- and paMAP-source gene signatures (saMAP ssGSEA and paMAP ssGSEA, respectively) showed a good correlation with the sternness signatures in an array of RNA-seq data from PSCs, sorted progenitor and differentiated cells from various sources (Pearson's R>0.5 for most gene sets, FIGS. 9D and E). Although additional analyses will be required to validate this finding in other datasets, the paMAP-source gene enrichment achieved the highest specificity to PSCs in our analyses (FIGS. 9F and G). Hence, it may be concluded that the immunopeptidome of iPSCs contains paMAPs derived from pluripotency-associated transcription events absent from healthy tissues and ASCs.

To assess the robustness of paMAP and saMAP identifications, the two best-in-class metrics for validation of MAPs identified with high-throughput MS were used. The distribution of the observed MAP retention times (RT) was compared with the distribution of the RT calculated using the DeepLC algorithm (Bouwmeester et al., 2020) and with the distribution of the hydrophobicity index assessed with SSRcalc (Krokhin, 2006). Both of these metrics had a strong correlation with the observed RTs for paMAPs and saMAPs (FIGS. 2G and H), and the RT distributions were not significantly different from the distribution of canonical proteome-derived peptides (F-test), supporting their correct identification.

### Example 4: paMAPs are Shared Across Cancer Types

It was next evaluated whether cancer cells present aberrant expression of paMAPs. To this end, the MCS of paMAPs in the RNA-seq of cancer samples from the 33 cancer types included in The Cancer Genome Atlas (TCGA) and from previous proteogenomic studies of acute myeloid leukemia (AML) (Ehx et al., 2021) and ovarian high-grade serous carcinoma (HGSC) (Zhao et al., 2020) were queried. It was found that 40 of the 46 paMAPs were expressed in at least 10% of the samples in up to 14 cancer types from TCGA, and 9 of these paMAPs were shared by more than 50% of the samples in one or two cancer types (FIG. 3). AML and HGSC samples studied in previous studies also shared expression of 13 and 19 paMAPs, respectively (FIG. 3). While most paMAPs were novel, six of them were previously reported in the context of cancer immunotherapy and shared by many TCGA cancer types (FIG. 3, bold). Of the reported paMAPs, five derive from in-frame exonic translation (Duffour et al., 1999; Huang et al., 1991; Jia et al., 2010; Schuster et al., 2017), whereas one derives from a 3′UTR and has been independently identified as an aeTSA in HGSC samples (Zhao et al., 2020) (Table 1B). Hence, novel commonly expressed paMAP-coding sequences have a high potential to generate shared TSAs between patients across multiple cancer types.

### Example 5: High-Stemness Cancers Acquire paMAP Expression

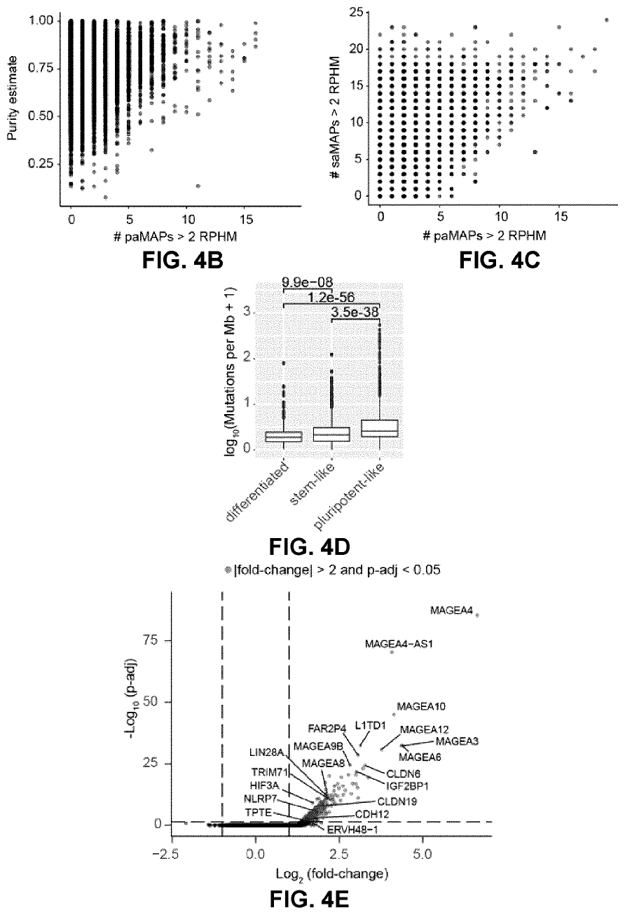

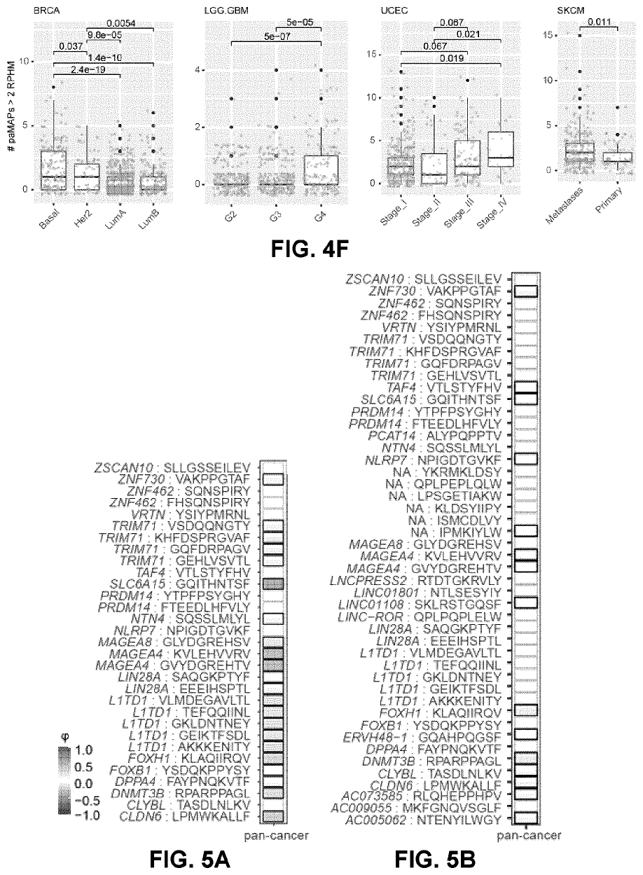

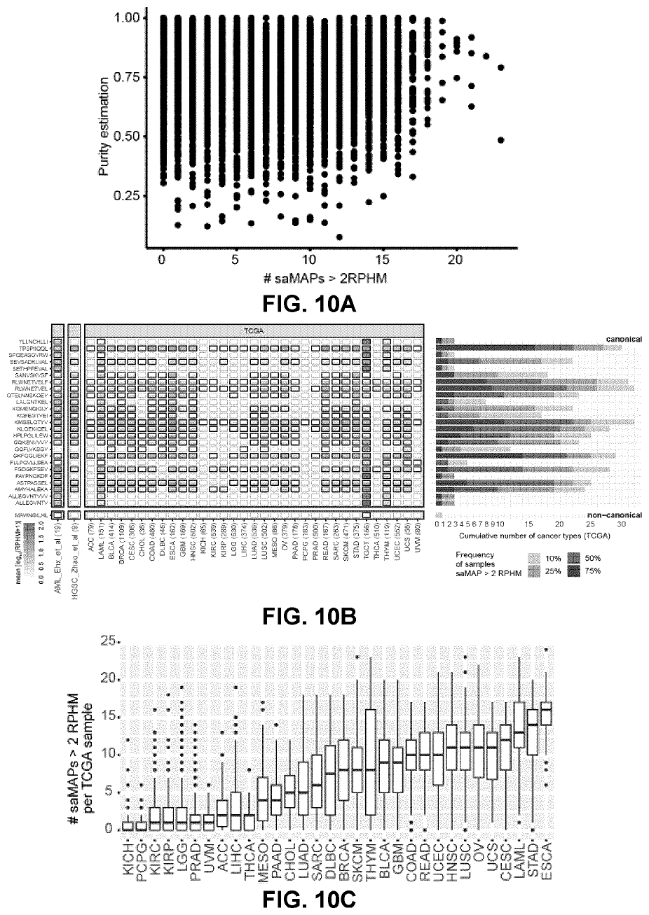

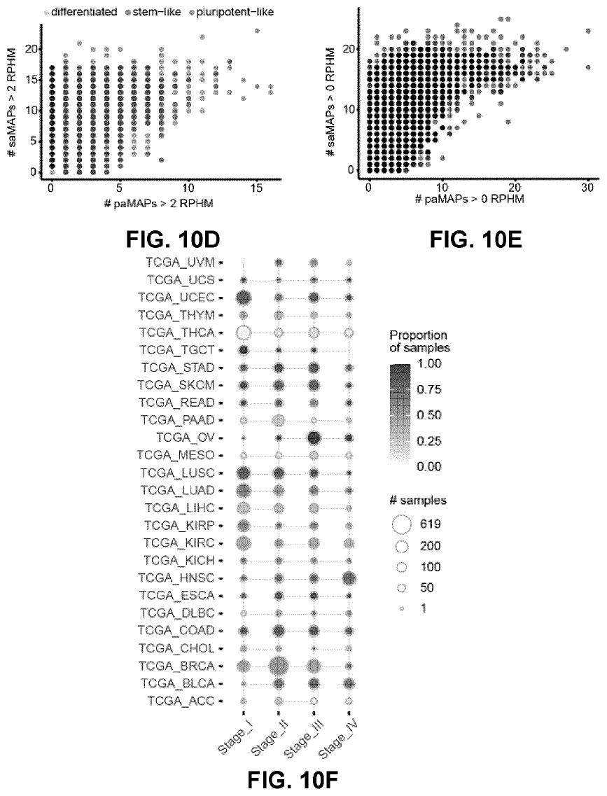

Consistent with previous reports showing that stemness varies across TCGA samples (Malta et al., 2018; Miranda et al., 2019; Smith et al., 2018), it was found that at the RNA level, the number of expressed paMAPs ranged from 0 to 19 per sample (excluding testis cancer, FIG. 4A). However, the inclusion of purity estimates for 21 solid cancers from Aran and colleagues (Aran et al., 2015) revealed that the number of expressed paMAPs increased with sample purity. From this correlation, it was inferred that paMAPs are specifically expressed in cancer cells and that a low purity may underestimate their number in some cancer samples (FIG. 4B). However, the situation was different with saMAPs, which had similar counts in high- and low-purity samples. The latter likely reflected expression of saMAPs in healthy or pre-cancerous adult stem cells or healthy proliferating cells (FIG. 10A).

Two additional observations could be made by comparing the expression of paMAPs and saMAPs: i) saMAPs were more widely expressed in cancer samples, with 86% of TCGA samples expressing at least one saMAP, and only 60% of samples expressing one paMAP or more (FIGS. 4A, 10B and 10C), and ii) paMAP expression co-occurred with saMAPs, but not all high-stemness samples were paMAP-positive, even when accounting for sample purity (FIGS. 4C, 10D and 10E). This suggests that paMAP expression appears with cancer progression and further dedifferentiation from stemness to a pluripotency-associated program. Indeed, a gradual increase in the non-synonymous mutation load in cancer samples from those with no paMAP/saMAP expression, to samples with saMAP expression only, followed by paMAP (and saMAP)-expressing samples (FIGS. 4D and 10D), was found. A differential gene expression analysis (controlling for purity and tumor type) was then performed between samples with paMAPs vs. those with many saMAPs but no paMAPs. This analysis revealed that tumors with paMAPs overexpressed genes involved in cancer cell migration and invasiveness, namely CDH12 (cadherin-12) and HIF3A (hypoxia-inducible factor 3 alpha subunit) (Ma et al., 2016; Wang et al., 2011; Zhou et al., 2018) (FIG. 4E and Table 2). High paMAP-expressing samples also showed overexpression of embryonic antigens and CGAs in addition to paMAP-source genes, including TPTE (transmembrane phosphatase with tensin homology) and MAGEA3 (melanoma-associated antigen A3). Notably, TPTE and MAGEA3 were reported to induce durable immune responses in patients with unresectable melanoma (Sahin et al., 2020).

TABLE 2
Related to FIG. 4E. Differentially expressed genes (p-adjusted <
0.05 and absolute fold change > 2) between TCGA samples with high
paMAP expression (>4 paMAPs, n = 775) and high saMAP expression
(>4 saMAPs and 0 paMAPs, n = 1270). TGCT was excluded from this analysis.

| Genes | logFC | AveExpr | t | P.Value | adj.P.Val |
|---|---|---|---|---|---|
| MAGEA4 | 6.632257708 | −3.462000504 | 21.19445572 | 1.61E−90 | 3.79E−86 |
| MAGEA3 | 4.40559481 | −3.428705246 | 12.84992453 | 1.09E−36 | 4.27E−33 |
| MAGEA6 | 4.366473622 | −3.580584508 | 12.8724832 | 8.29E−37 | 4.06E−33 |
| MAGEA10 | 4.137307765 | −4.335730041 | 15.17230062 | 1.24E−49 | 9.76E−46 |
| MAGEA4-AS1 | 4.071719197 | −4.9605578 | 19.10201332 | 3.12E−75 | 3.68E−71 |
| MAGEA12 | 3.769224988 | −3.78369304 | 12.53441864 | 4.54E−35 | 1.53E−31 |
| MAGEA11 | 3.376663645 | −3.462693839 | 9.982287754 | 3.05E−23 | 4.24E−20 |
| CLDN6 | 3.272543706 | −1.726222796 | 11.15963171 | 2.08E−28 | 4.91E−25 |
| CSAG1 | 3.210914194 | −2.986166987 | 10.86105226 | 4.74E−27 | 1.02E−23 |
| L1TD1 | 3.131383884 | −1.448928921 | 12.86938508 | 8.60E−37 | 4.06E−33 |
| CLCA1 | 3.068669547 | −4.071871582 | 7.668347052 | 1.34E−14 | 8.13E−12 |
| FAR2P4 | 3.063473209 | −3.97969639 | 12.06831087 | 9.75E−33 | 2.88E−29 |
| MAGEA1 | 3.051093058 | −4.023098803 | 8.895077678 | 6.31E−19 | 6.21E−16 |
| LINC02864 | 3.013716529 | −4.167809677 | 10.56482125 | 9.80E−26 | 1.93E−22 |
| CTCFL | 3.00057319 | −3.120008804 | 9.333453001 | 1.30E−20 | 1.46E−17 |
| IGF2BP1 | 3.000223209 | −0.576086822 | 10.27422162 | 1.78E−24 | 3.24E−21 |
| LIN28B | 2.899417656 | −4.041691181 | 9.385692399 | 8.09E−21 | 1.01E−17 |
| FAR2P1 | 2.859736191 | −1.523452552 | 8.202138437 | 2.07E−16 | 1.69E−13 |
| MAGEA9B | 2.820360115 | −5.172591831 | 11.20912989 | 1.23E−28 | 3.22E−25 |
| LINC00648 | 2.77908636 | −3.550632196 | 10.25568986 | 2.14E−24 | 3.61E−21 |
| DSCR8 | 2.684786789 | −4.312165986 | 8.604230676 | 7.56E−18 | 6.62E−15 |
| CTAG2 | 2.620806715 | −4.924562108 | 8.053869198 | 6.77E−16 | 5.00E−13 |
| CSAG3 | 2.609891556 | −4.052699875 | 9.103912412 | 1.01E−19 | 1.09E−16 |
| LINC01194 | 2.570030064 | −5.183448976 | 10.02757135 | 1.97E−23 | 2.91E−20 |
| MAGEC2 | 2.540968607 | −4.426898723 | 7.311856552 | 1.89E−13 | 9.09E−11 |
| LINC00470 | 2.487864593 | −3.906617783 | 8.831086074 | 1.10E−18 | 1.04E−15 |
| CSAG2 | 2.454911175 | −4.583016437 | 9.357353367 | 1.05E−20 | 1.24E−17 |
| MAGEB2 | 2.451063532 | −4.800494947 | 7.919267771 | 1.95E−15 | 1.25E−12 |
| GABRA3 | 2.401202674 | −1.5386807 | 6.804133986 | 6.67E−12 | 2.72E−09 |
| LINC00355 | 2.377250775 | −4.544278065 | 8.023087323 | 8.64E−16 | 6.00E−13 |
| AP005262.2 | 2.361954746 | −4.841835769 | 9.094714856 | 1.10E−19 | 1.13E−16 |
| CT45A10 | 2.338296719 | −5.166077827 | 8.546342432 | 1.23E−17 | 1.04E−14 |
| SLCO1A2 | 2.33225708 | −1.820708837 | 7.093914045 | 8.97E−13 | 4.15E−10 |
| CLEC2L | 2.318228974 | −2.559416528 | 7.465917948 | 6.11E−14 | 3.14E−11 |
| SAGE1 | 2.278987893 | −4.599785502 | 7.742377529 | 7.64E−15 | 4.75E−12 |
| TRIM71 | 2.274783038 | −2.315646179 | 7.382139637 | 1.13E−13 | 5.68E−11 |
| MAGEC1 | 2.27217942 | −4.26256943 | 6.604329461 | 2.54E−11 | 9.44E−09 |
| G2E3-AS1 | 2.253302461 | −4.544466185 | 8.033770877 | 7.94E−16 | 5.68E−13 |
| MKRN3 | 2.243967386 | −1.621335464 | 6.559343815 | 3.42E−11 | 1.22E−08 |
| POU6F2 | 2.206660524 | −2.269357531 | 5.513569641 | 1.98E−08 | 5.04E−06 |
| AC008443.3 | 2.204775859 | −4.687607745 | 10.15931004 | 5.50E−24 | 8.66E−21 |
| LINC01446 | 2.177749055 | −4.950349114 | 7.617990652 | 1.96E−14 | 1.13E−11 |
| SLC6A10P | 2.152364261 | −4.15168278 | 6.529547371 | 4.16E−11 | 1.44E−08 |
| AF279873.3 | 2.151188888 | −4.9621382 | 7.654738094 | 1.49E−14 | 8.79E−12 |
| FAM133A | 2.149524362 | −2.821149844 | 7.93907454 | 1.67E−15 | 1.13E−12 |
| CLDN19 | 2.140875981 | −3.395891462 | 6.603408639 | 2.56E−11 | 9.44E−09 |
| NAA11 | 2.137617879 | −5.145381142 | 7.587314854 | 2.47E−14 | 1.36E−11 |
| GTSF1 | 2.130758946 | −1.263445408 | 6.927779093 | 2.86E−12 | 1.21E−09 |
| LIN28A | 2.121571969 | −4.499735739 | 7.927629467 | 1.83E−15 | 1.20E−12 |
| MAGEA8 | 2.117907004 | −3.977326437 | 8.179179909 | 2.49E−16 | 1.96E−13 |
| LINC01980 | 2.117761253 | −3.829927052 | 5.326175055 | 5.57E−08 | 1.34E−05 |
| KLF2P4 | 2.100589415 | −4.750235714 | 8.686309098 | 3.78E−18 | 3.43E−15 |
| DSCR4 | 2.092755232 | −5.403808431 | 8.086299791 | 5.23E−16 | 3.99E−13 |
| ITLN1 | 2.08867667 | −2.29318729 | 5.034721846 | 2.60E−07 | 5.54E−05 |
| CT45A1 | 2.079380874 | −5.405359006 | 7.314023296 | 1.86E−13 | 9.09E−11 |
| NF1P8 | 2.075370511 | −5.692894919 | 9.473936792 | 3.61E−21 | 4.74E−18 |
| KLF2P1 | 2.073008444 | −3.6200697 | 6.272089566 | 2.17E−10 | 7.02E−08 |
| MIR548XHG | 2.070444283 | −5.331794313 | 7.517282795 | 4.17E−14 | 2.24E−11 |
| LINC02575 | 2.062530546 | −3.044269717 | 6.406424148 | 9.23E−11 | 3.12E−08 |
| AC011632.1 | 2.051172936 | −3.638753537 | 5.723052686 | 6.01E−09 | 1.63E−06 |
| PAGE2 | 2.049576354 | −4.909073568 | 5.880589409 | 2.38E−09 | 6.71E−07 |
| LGSN | 2.031054352 | −3.044695186 | 6.366870951 | 1.19E−10 | 3.90E−08 |
| AACSP1 | 2.0309077 | −3.709092597 | 6.62325996 | 2.24E−11 | 8.69E−09 |
| PHF2P2 | 2.026083677 | −5.004988879 | 7.085995504 | 9.49E−13 | 4.31E−10 |
| CCNYL2 | 2.022669698 | −3.765568365 | 6.796776103 | 7.01E−12 | 2.81E−09 |
| LINC00668 | 2.014493906 | −1.56612255 | 4.388109294 | 6.01E−06 | 0.0010069 |
| LINC01518 | 2.006505824 | −5.085290655 | 7.023286174 | 1.47E−12 | 6.44E−10 |
| ADAMTS20 | 2.005697318 | −3.637561693 | 6.502598778 | 4.96E−11 | 1.70E−08 |
| SOHLH1 | 2.001796546 | −4.330101622 | 6.620743 | 2.28E−11 | 8.69E−09 |
| PNMA5 | 1.999933453 | −3.517960398 | 6.149693248 | 4.66E−10 | 1.41E−07 |
| DEFA5 | 1.993805613 | −4.907030767 | 5.122178725 | 1.65E−07 | 3.68E−05 |
| LINC02820 | 1.991763316 | −4.143824928 | 6.073126976 | 7.47E−10 | 2.21E−07 |
| BAGE2 | 1.988152739 | −5.263687596 | 6.723853937 | 1.15E−11 | 4.51E−09 |
| LINC02241 | 1.973341777 | −5.300067715 | 7.028904879 | 1.42E−12 | 6.31E−10 |
| AL117329.1 | 1.954040172 | −3.347929769 | 5.428064907 | 3.19E−08 | 8.01E−06 |
| LINC02315 | 1.951004763 | −4.212787021 | 5.581207509 | 1.35E−08 | 3.56E−06 |
| EZHIP | 1.946635639 | −4.861354709 | 7.127286536 | 7.08E−13 | 3.35E−10 |
| ASNSP1 | 1.921871347 | −4.78892165 | 6.562735605 | 3.34E−11 | 1.22E−08 |
| GPR1-AS | 1.908543421 | −5.109281461 | 6.899594108 | 3.47E−12 | 1.44E−09 |
| TMEM132D- | 1.903229597 | −5.300638941 | 6.166644799 | 4.20E−10 | 1.29E−07 |
| AS1 | | | | | |
| AC132807.2 | 1.89850573 | −5.072021979 | 6.403870579 | 9.39E−11 | 3.12E−08 |
| AC006206.2 | 1.894667534 | −2.638052905 | 4.830565632 | 7.32E−07 | 0.000146474 |
| COX7B2 | 1.891177775 | −5.455245962 | 5.775282699 | 4.43E−09 | 1.22E−06 |
| AC106771.1 | 1.88506669 | −5.675456684 | 7.591935805 | 2.39E−14 | 1.34E−11 |
| HIF3A | 1.870703661 | 0.701127594 | 6.55459978 | 3.53E−11 | 1.24E−08 |
| GALNT8 | 1.870085433 | −0.437607776 | 6.112392964 | 5.87E−10 | 1.75E−07 |
| ZIC1 | 1.86070947 | −1.516130927 | 3.956403738 | 3.94E−05 | 0.005714133 |
| AL139023.1 | 1.854951928 | −5.012681672 | 6.242786699 | 2.61E−10 | 8.11E−08 |
| HTR2C | 1.854221034 | −4.119297399 | 5.17313745 | 1.26E−07 | 2.87E−05 |
| NLRP7 | 1.849146069 | −2.338087652 | 5.569520605 | 1.45E−08 | 3.76E−06 |
| PRSS21 | 1.840730424 | −0.204247118 | 3.492452745 | 0.000244428 | 0.031378452 |
| BRDT | 1.835299037 | −3.904248712 | 5.048091818 | 2.43E−07 | 5.22E−05 |
| CSAG4 | 1.834311032 | −5.614686507 | 7.492842104 | 5.00E−14 | 2.63E−11 |
| LINC01234 | 1.826375942 | −1.805925328 | 5.261515118 | 7.90E−08 | 1.83E−05 |
| HBE1 | 1.817602419 | −4.603140502 | 6.063344666 | 7.93E−10 | 2.31E−07 |
| PURPL | 1.814486412 | −3.679257662 | 5.553786864 | 1.58E−08 | 4.06E−06 |
| LINC00221 | 1.809652245 | −5.175454703 | 5.108685068 | 1.77E−07 | 3.92E−05 |
| LINC02616 | 1.805480685 | −5.362176958 | 6.26329133 | 2.29E−10 | 7.32E−08 |
| LCN15 | 1.799417681 | −3.762435269 | 3.990978476 | 3.41E−05 | 0.005125976 |
| AC021504.1 | 1.798373774 | −3.676444658 | 4.397046128 | 5.77E−06 | 0.000973618 |
| AC010789.1 | 1.794363716 | −4.853215323 | 5.683203597 | 7.57E−09 | 2.03E−06 |
| PASD1 | 1.791445721 | −5.484613287 | 6.249711356 | 2.50E−10 | 7.87E−08 |
| REG3A | 1.791298501 | −4.368125337 | 3.809502388 | 7.17E−05 | 0.010020783 |
| AC073365.1 | 1.783581497 | −4.131354349 | 4.658370999 | 1.70E−06 | 0.000313164 |
| MAGEB1 | 1.779675232 | −5.606739837 | 5.780225608 | 4.31E−09 | 1.20E−06 |
| SLC5A12 | 1.766601958 | −1.94255727 | 4.535815865 | 3.04E−06 | 0.000543406 |
| RSPO4 | 1.757385421 | −1.302683289 | 4.506912915 | 3.48E−06 | 0.000612784 |
| AC209154.1 | 1.748432458 | −5.516016104 | 5.935839866 | 1.71E−09 | 4.94E−07 |
| LINC01667 | 1.746014554 | −5.061254566 | 4.960683919 | 3.80E−07 | 7.95E−05 |
| AL121949.1 | 1.744994872 | −4.316419882 | 5.307422742 | 6.17E−08 | 1.44E−05 |
| AC010894.3 | 1.74025515 | −2.833927786 | 4.858999407 | 6.35E−07 | 0.000128226 |
| AP005119.2 | 1.729259502 | 4.88170109 | 5.881961385 | 2.37E−09 | 6.71E−07 |
| MKRN9P | 1.722634321 | −4.182416635 | 5.35370364 | 4.80E−08 | 1.19E−05 |
| AC010595.1 | 1.715354293 | −4.644368767 | 4.771730953 | 9.78E−07 | 0.000191 |
| AL133467.2 | 1.701148374 | −4.976525616 | 5.195371255 | 1.12E−07 | 2.58E−05 |
| LINC02476 | 1.70039512 | −5.644032437 | 6.986242863 | 1.91E−12 | 8.19E−10 |
| RETNLB | 1.699054388 | −3.885915433 | 4.77330013 | 9.71E−07 | 0.000191 |
| KCNMB2-AS1 | 1.695060685 | −0.988145978 | 3.668075209 | 0.000125341 | 0.016822024 |
| XAGE2 | 1.686682886 | −4.710971025 | 4.198307223 | 1.40E−05 | 0.002253968 |
| AC090015.1 | 1.685630751 | −4.398114868 | 4.383254941 | 6.14E−06 | 0.001022144 |
| KIF1A | 1.682775711 | 1.285541005 | 3.71448129 | 0.000104563 | 0.014276715 |
| AC007848.1 | 1.678355546 | −4.038653977 | 4.587460571 | 2.38E−06 | 0.000432515 |
| KIF25-AS1 | 1.674035709 | −3.690845996 | 3.761577794 | 8.68E−05 | 0.011992342 |
| FBN3 | 1.673332831 | −0.814673664 | 4.059401701 | 2.55E−05 | 0.003916339 |
| SELENOOLP | 1.67332344 | −5.427826167 | 5.314850091 | 5.92E−08 | 1.41E−05 |
| AL133467.4 | 1.665939518 | −5.219430378 | 5.339934719 | 5.17E−08 | 1.26E−05 |
| LINC00858 | 1.658600111 | −2.959261909 | 3.7390908 | 9.49E−05 | 0.013033171 |
| PSLNR | 1.655090937 | −4.913943477 | 4.650276408 | 1.76E−06 | 0.000323051 |
| SLC9A3P2 | 1.64973031 | −5.075829423 | 4.891592777 | 5.39E−07 | 0.000110812 |
| AC090809.1 | 1.649279516 | −5.541899357 | 5.050597803 | 2.40E−07 | 5.20E−05 |
| FAM230C | 1.644250938 | −5.449349021 | 4.826806663 | 7.46E−07 | 0.000147979 |
| POU6F2-AS2 | 1.643568827 | −4.777156529 | 4.418726223 | 5.23E−06 | 0.000888086 |
| GABRG2 | 1.636905162 | −4.549333623 | 3.813010229 | 7.07E−05 | 0.009939381 |
| AC092969.1 | 1.630922051 | −3.497782673 | 3.98185434 | 3.54E−05 | 0.005225389 |
| AP005205.3 | 1.630886594 | −4.902468701 | 5.619686812 | 1.09E−08 | 2.89E−06 |
| LINC01807 | 1.630635481 | −4.415451565 | 5.310606124 | 6.06E−08 | 1.43E−05 |
| SMPD4P1 | 1.625801064 | −4.11531327 | 5.170845648 | 1.28E−07 | 2.88E−05 |
| AP003900.1 | 1.622656777 | −5.509143568 | 5.344539782 | 5.04E−08 | 1.24E−05 |
| LINC01297 | 1.616129017 | −5.187259869 | 5.066954977 | 2.20E−07 | 4.82E−05 |
| STRA8 | 1.613442216 | −4.32985331 | 4.584071687 | 2.42E−06 | 0.00043616 |
| LINC00898 | 1.612387192 | −4.234760239 | 4.105468013 | 2.10E−05 | 0.003281081 |
| AC104041.1 | 1.611665378 | −4.278463036 | 4.261121772 | 1.06E−05 | 0.001744642 |
| GPR158 | 1.60678108 | 0.014262028 | 3.983731086 | 3.51E−05 | 0.005217201 |
| DPPA2 | 1.602918602 | −5.428860929 | 4.482048686 | 3.90E−06 | 0.000682839 |
| FSTL5 | 1.601480651 | −3.657873756 | 3.656693993 | 0.000130998 | 0.017481985 |
| LINC01419 | 1.599020669 | −5.434029513 | 4.276547005 | 9.93E−06 | 0.001640486 |
| ZNF280A | 1.597023609 | −3.745846628 | 4.693270103 | 1.43E−06 | 0.000268866 |
| LINC01257 | 1.596239471 | −4.822384088 | 4.892903134 | 5.36E−07 | 0.000110812 |
| DDX53 | 1.593380226 | −5.585676236 | 4.973146785 | 3.57E−07 | 7.53E−05 |
| LINC02109 | 1.587708091 | −5.096060193 | 4.681288192 | 1.52E−06 | 0.000282649 |
| ATOH1 | 1.584011759 | −4.323628236 | 3.955916762 | 3.94E−05 | 0.005714133 |
| GAGE2A | 1.578825519 | −5.784159759 | 4.515053007 | 3.35E−06 | 0.000594373 |
| AC061975.6 | 1.576953457 | −4.390879942 | 4.134403962 | 1.85E−05 | 0.00293577 |
| MIR663AHG | 1.571963491 | −4.295677555 | 3.892532369 | 5.12E−05 | 0.007330284 |
| ZNF716 | 1.571903781 | −5.225870114 | 4.222355442 | 1.26E−05 | 0.002042133 |
| LINC02432 | 1.571423007 | −2.466053998 | 3.518150827 | 0.000222066 | 0.028663515 |
| CACNA1B | 1.570321509 | −1.022319545 | 3.700942065 | 0.000110263 | 0.014968583 |
| CDH12 | 1.570161085 | −3.529294838 | 3.692857345 | 0.000113806 | 0.015361157 |
| AC074389.2 | 1.563466892 | −5.315547276 | 4.045917975 | 2.70E−05 | 0.004120084 |
| OR51B5 | 1.563349158 | −4.863088039 | 4.722123012 | 1.25E−06 | 0.00023945 |
| LINC01456 | 1.561696286 | −4.890259711 | 4.194193505 | 1.43E−05 | 0.002279394 |
| TEX15 | 1.559891315 | −3.253834105 | 3.942966415 | 4.16E−05 | 0.00599251 |
| RPL18P13 | 1.556036274 | −3.569081847 | 4.70484374 | 1.36E−06 | 0.000258311 |
| VCX | 1.554997667 | −4.658496433 | 4.071681717 | 2.42E−05 | 0.003741331 |
| LINC01854 | 1.549904135 | −5.575379569 | 4.764491129 | 1.01E−06 | 0.000196286 |
| MED15P9 | 1.549577519 | −5.311556643 | 4.454791012 | 4.43E−06 | 0.000763344 |
| LINC02327 | 1.547515032 | −5.650900283 | 4.700194283 | 1.39E−06 | 0.000262083 |
| PAGE1 | 1.538121868 | −5.476489549 | 3.987165156 | 3.46E−05 | 0.005175416 |
| CNTNAP4 | 1.53725007 | −3.991211492 | 3.579106389 | 0.000176434 | 0.022898668 |
| AC005537.2 | 1.529531157 | −2.609421265 | 3.762626428 | 8.65E−05 | 0.011992342 |
| AL157778.1 | 1.52500822 | −5.636437717 | 4.439131102 | 4.76E−06 | 0.000814521 |
| PRR20G | 1.513853506 | −5.165353937 | 4.124894759 | 1.93E−05 | 0.003038285 |
| LINC02335 | 1.504549909 | −5.765093178 | 4.252541932 | 1.10E−05 | 0.001799751 |
| CT55 | 1.498663122 | −5.137051168 | 4.878510915 | 5.76E−07 | 0.000117308 |
| ERVV-2 | 1.496551816 | −4.750807631 | 3.394488102 | 0.000350402 | 0.044261255 |
| MDGA2 | 1.494335611 | −3.870539531 | 3.853890914 | 5.99E−05 | 0.008477478 |
| AL031736.1 | 1.460455029 | −4.549830006 | 3.979497954 | 3.57E−05 | 0.005244266 |
| LINC02267 | 1.460238319 | −5.874832643 | 4.465983619 | 4.20E−06 | 0.000730198 |
| MAGEA9 | 1.449190159 | −5.915870849 | 4.096770666 | 2.18E−05 | 0.003383303 |
| ERVH48-1 | 1.448417931 | −2.632076296 | 3.613279908 | 0.000154854 | 0.02043473 |
| LINC02377 | 1.443868796 | −5.646432241 | 3.602484002 | 0.000161389 | 0.021061714 |
| POTEF | 1.440127985 | −2.58034864 | 4.042167393 | 2.75E−05 | 0.004159188 |
| CASC20 | 1.434497386 | −5.072988283 | 3.383790461 | 0.000364262 | 0.045767251 |
| TPTE | 1.416897708 | −5.806978532 | 3.87115525 | 5.59E−05 | 0.007950587 |
| GOLGA6L7 | 1.416436192 | −4.741969567 | 3.607620804 | 0.000158248 | 0.020766565 |
| IZUMO2 | 1.406965848 | −4.762766617 | 3.465128312 | 0.000270499 | 0.034537638 |
| LINC01901 | 1.406127948 | −4.999833531 | 3.430111356 | 0.000307705 | 0.039076867 |
| LINC02484 | 1.36981357 | −5.93251048 | 3.616771196 | 0.000152795 | 0.020276217 |

Supporting the notion that pluripotency is associated with cancer progression and invasiveness (Ben-Porath et al., 2008), paMAPs were preferentially expressed in cancer subtypes with poor prognosis or advanced stages (FIG. 4F). In breast cancer (BRCA), the basal subtype had the highest number of paMAPs, followed by the HER2 subtype and the luminal A and luminal B subtypes. Glioblastoma (GBM, G4) samples also showed a significantly higher number of paMAPs compared to low-grade gliomas (LGG, G2, and G3), while stage III and IV endometrial cancers (UCEC) expressed more paMAPs than early-stage tumors. Similarly, metastatic melanoma samples expressed more paMAPs compared to primary lesions (FIG. 4F). Nonetheless, it was observed that cancers from all subtypes and stages could re-express paMAPs (FIGS. 4F and 10F), indicating that immune targeting of paMAPs could be envisioned at any tumor stage.

### Example 6: Shared Epigenetic and Signaling Events Associate with paMAP and saMAP Expression Across Cancers

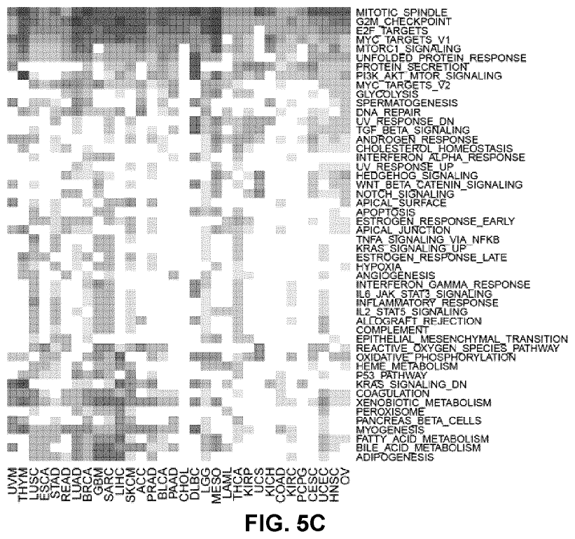

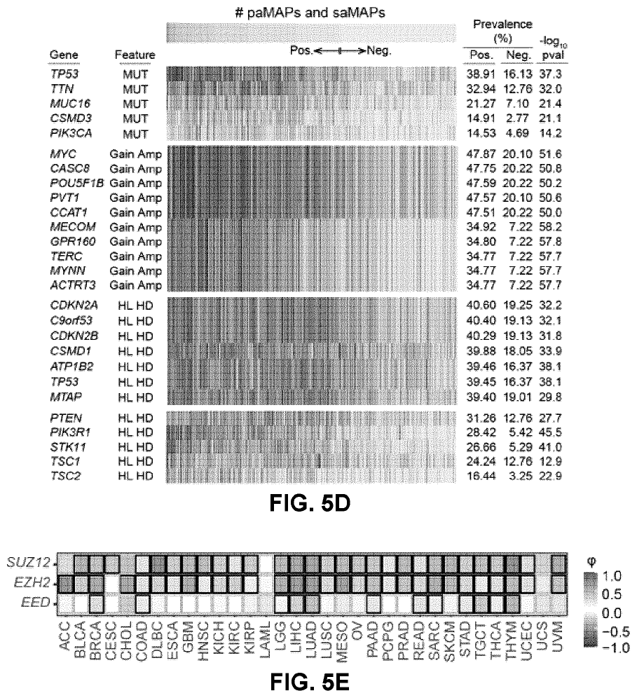

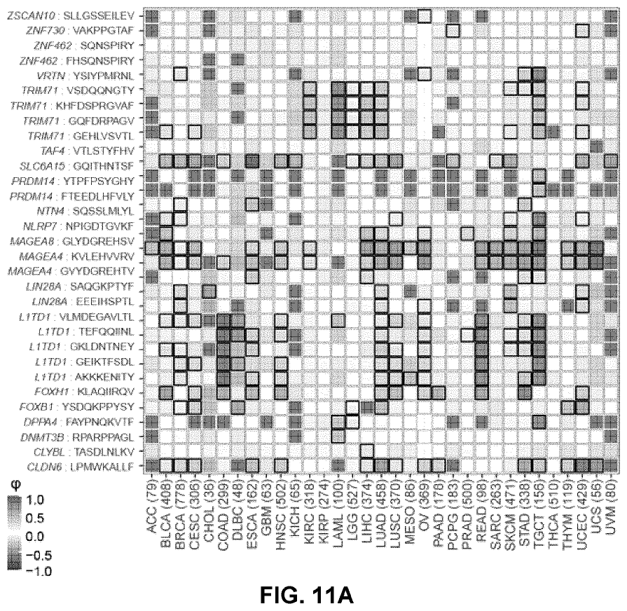

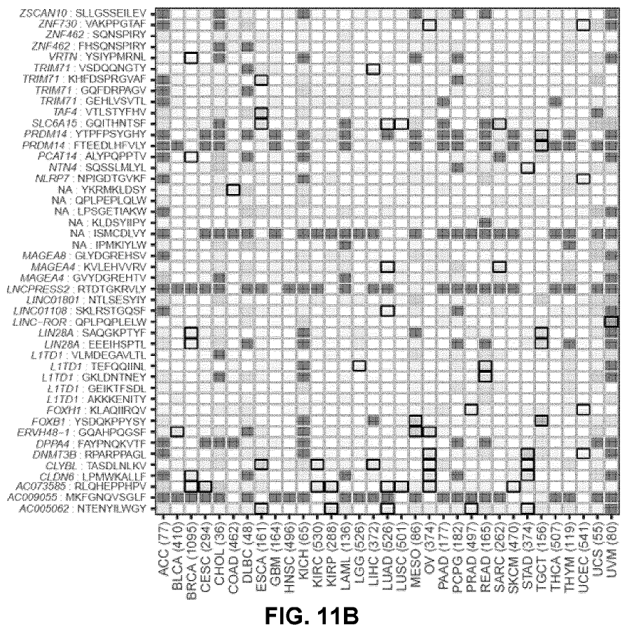

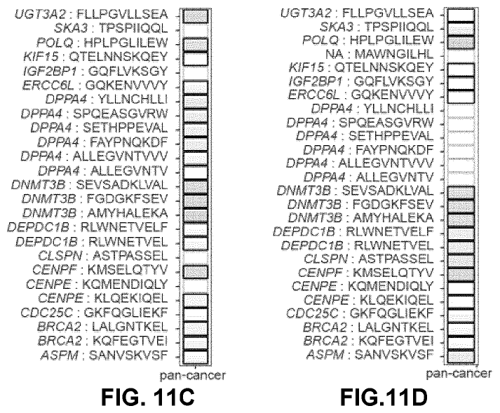

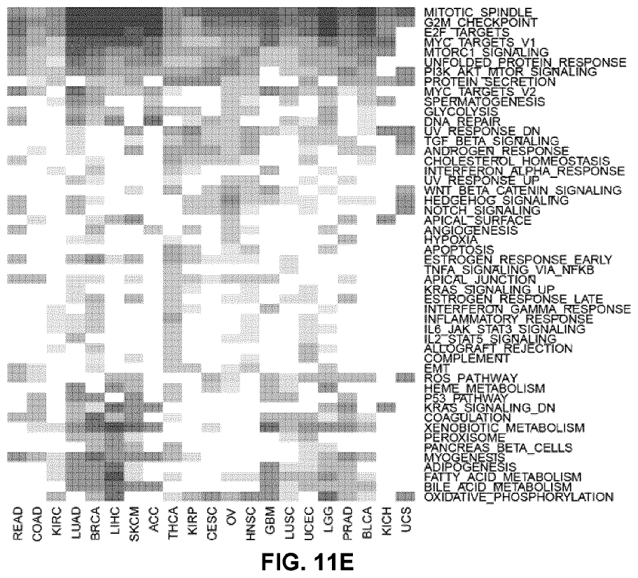

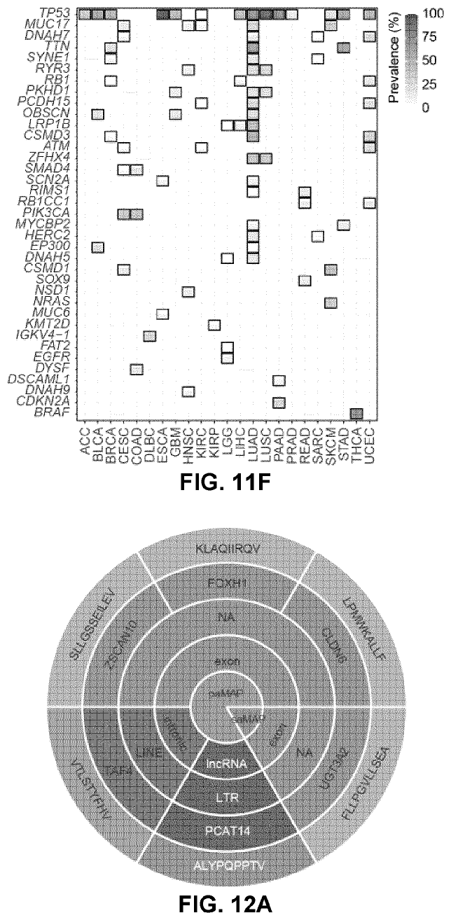

To elucidate the mechanisms regulating paMAP expression, its potential correlation with epigenetic and focal DNA copy-number aberrations was first evaluated. This analysis showed that, within and across cancer types, the DNA methylation status at source-gene promoter regions negatively correlated with the MCS expression for most paMAPs (FIGS. 5A and 11A). In contrast, only a small fraction of paMAPs was associated with an increase in DNA copy number (FIGS. 5B and 11B). A similar trend was observed for saMAPs (FIGS. 11C and D), indicating that, irrespective of the tissue of origin, epigenetics is an important mechanism in acquiring stemness and pluripotency features in cancer.

Next, the hallmark gene set collection from the Molecular Signature Database (MSigDB) (Liberzon et al., 2015) was used to explore other events that may drive paMAP and saMAP expression (interrogated together given their co-occurrence) and to understand their effect on global patterns. First, this data revealed that proliferation-related gene sets such as mitotic spindle assembly, G2/M checkpoint, E2F, and MYC signaling, were the most enriched and shared across all paMAP and saMAP-expressing samples (FIGS. 5C and 11E). In accordance with the high demands of proliferation and functionality, DNA repair, protein synthesis, and the unfolded protein response programs were also robustly upregulated. A second PSC pattern correlated with paMAP and saMAP expression within cancers: metabolic rewiring to glycolysis and downregulation of pathways active in differentiated tissues (i.e., oxidative phosphorylation, bile acid, and fatty acid metabolism) (Aran et al., 2017a; Kroemer and Pouyssegur, 2008; Zhang et al., 2012).

Notably, two signaling pathways were highly enriched in paMAP and saMAP-expressing samples: MYC signaling and the phosphoinositide 3-kinase (PI3K)/protein kinase B (AKT)/mammalian target of rapamycin (mTOR) pathway. These two pathways regulate cellular growth, protein synthesis, and metabolism to promote the survival and dissemination of cancer cells (Heiden et al., 2009; Janku et al., 2018) (FIG. 5C). Though activating alterations in these pathways are the most frequent among TCGA samples (Sanchez-Vega et al., 2018), their prevalence in paMAP and saMAP-expressing samples was significantly higher than in non-expressing samples. This was particularly true for mutations in PIK3CA, deletions in PI3K/AKT signaling antagonists PTEN, PIK3R1, and STK11 (also called LKB1), and MYC amplification (FIG. 50). The activating PIK3CA^{H1047R} mutation induces multipotency by dedifferentiation in mouse models of breast, lung, and colorectal cancer, consistent with the ability of PI3K/AKT activation to increase the expression of pluripotency genes and self-renewal in human PSCs (Madsen, 2020). Moreover, the link between metabolism and epigenetics under the regulation of MYC and PI3K/AKT/mTOR pathways has been described in both PSCs and cancer (Dai et al., 2020; Fagnocchi and Zippo, 2017; Madsen, 2020; Zhang et al., 2012). In addition to promoting cell growth and proliferation, MYC overexpression induces transcriptional repression of lineage-specifying transcription factors. This is achieved via upregulation and recruitment of chromatin modifiers like the Polycomb repressive complex 2 (PRC2), which promotes epigenetic reprogramming towards a stem-like state, tumorigenesis, and self-renewal (Dardenne et al., 2016; Das et al., 2019; Fagnocchi and Zippo, 2017; Poli et al., 2018; Stine et al., 2015; Zhang et al., 2019). Accordingly, it was found that the number of paMAPs and saMAPs strongly correlated with the expression of PRC2 components (SUZ12, EZH2, EED) within cancers (FIG. 5E).

Other core signaling pathways that cross-talk to promote oncogenic dedifferentiation, namely Hedgehog, transforming growth factor (TGF)-β, WNT/β-catenin, and NOTCH signaling (Madsen, 2020; Malta et al., 2018; Pelullo et al., 2019), were also enriched in high paMAP and saMAP-expressing samples (FIG. 5C), whereas tumor suppressors had a high prevalence of deletions (FIG. 5D). Among them, it was found that the pluripotency inhibitor TP53 (Lin and Lin, 2017; Merkle et al., 2017) had a strong negative enrichment signature and the highest prevalence of mutations within and across cancers with paMAP and saMAP expression (FIGS. 5C, 5D and 11F). Altogether, these data indicate that common genomic and signaling aberrations cooperate to induce a unifying PSC-like program across cancers.

### Example 7: Immunogenicity of paMAPs and saMAPs

Given that paMAPs are appealing targets for immunotherapy, their immunogenicity was tested using in vitro T cell assays with peripheral blood mononuclear cells (PBMCs) from healthy donors. paMAPs were prioritized based on four criteria: i) the immunogenicity score predicted by Repitope, a machine learning algorithm that uses public T-cell receptor (TCR) databases to predict a probability of T cell response (Ogishi and Yotsuyanagi, 2019), ii) the HLA allotype presenting the paMAP (HLAA*02:01 or HLA-B*53:01 shared by the iPSCs and PBMCs donors), iii) expression in minimum 10% of the samples in at least one TOGA cancer type (FIG. 3), and iv) novel MAP status. The 008 T cell response against 11 paMAPs was tested using peptide-HLA tetramer staining and/or more sensitive functional expansion of specific T cells (FEST) assays (Danilova et al., 2018). In FEST assays, TCR sequencing is performed on T cells stimulated or not with synthetic paMAPs. T cells were stimulated in vitro with autologous T cell-depleted PBMCs pulsed with individual or pooled (n=5 or 6) paMAPs (Tables 3A-B).

TABLE 3A
peptides used in immunogenicity studies

| Pool of all | | | | |
|---|---|---|---|---|
| paMAPs HLA- | Pool of 5 | Pool of saMAPs | | |
| A*02:01 | paMAPs HLA- | (3 HLA-A*02:01 | HLA-B*53:01 | MelanA ctl |
| (6 peptides) | B*53:01 | 2 HLA-B*53:01) | peptide alone | peptide |
| GQFDRPAGV | QPLPQPLELW | HPLPGLILEW | LPMWKALLF | ELAGIGILTV |
| (pa_03) | (pa_01) | (sa_01) | (pa_06) | |
| RLQHEPPHPV | QPLPEPLQLW | ALLEGVNTV | | |
| (pa_05) | (pa_02) | (sa_02) | | |
| KLAQIIRQV | IPMKIYLW | FLLPGVLLSEA | | |
| (pa_07) | (pa_04) | (sa_04) | | |
| VTLSTYFHV | LPMWKALLF | TPSPIIQQL | | |
| (pa_10) | (pa_06) | (sa_05) | | |
| ALYPQPPTV | LPSGETIAKW | RLWNETVEL | | |
| (pa_11) | (pa_08) | (sa_06) | | |
| SLLGSSEILEV | | | | |
| (pa_12) | | | | |

TABLE 3B
TCRß clonotypes amplified by the peptides or peptide pools

| TCRß clonotype (TCRß CDR3 sequence) | |
|---|---|
| amplified | Peptide or peptide pool |
| CASSQPPAAGAKNIQYF (SEQ ID NO: 73) | Pool paMAPs HLA-A*02:01 |
| CASSITGGEKLFF (SEQ ID NO: 74) | Pool paMAPs HLA-A*02:01 |
| CASSKWGSYGYTF (SEQ ID NO: 75) | pa_10 peptide HLA-A*02:01 |
| CASSLPDTTYEQYF (SEQ ID NO: 76) | pa_10 peptide HLA-A*02:01 |
| CASSYRTGSAEAFF (SEQ ID NO: 77) | pa_11 peptide HLA-A*02:01 |
| CASSLGGAYEQYF (SEQ ID NO: 78) | pa_11 peptide HLA-A*02:01 |
| CASSYPQGGEQFF (SEQ ID NO: 79) | pa_11 peptide HLA-A*02:01 |
| CATSRGTGVNTEAFF (SEQ ID NO: 80) | pa_11 peptide HLA-A*02:01 |
| CASSSRTDTYSPLHF (SEQ ID NO: 81) | pa_12 peptide HLA-A*02:01 |
| CASSLGRWNSPLHF (SEQ ID NO: 82) | pa_12 peptide HLA-A*02:01 |
| CASSGPGDQPQHF (SEQ ID NO: 83) | pa_06 HLA-B*53:01 |
| CASSEWGSRAGGGDTQYF (SEQ ID NO: 84) | pa_06 HLA-B*53:01 |
| CASSMGRGHEQYF (SEQ ID NO: 85) | MelanA ctl peptide |
| CASSHFGRRGTEAFF (SEQ ID NO: 86) | MelanA ctl peptide |
| CASSSHLPYPYEQYF (SEQ ID NO: 87) | MelanA ctl peptide |

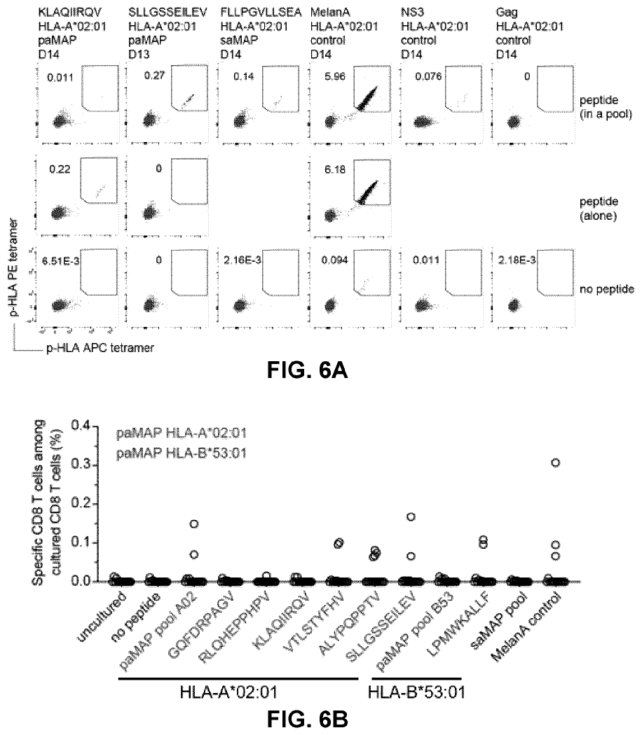

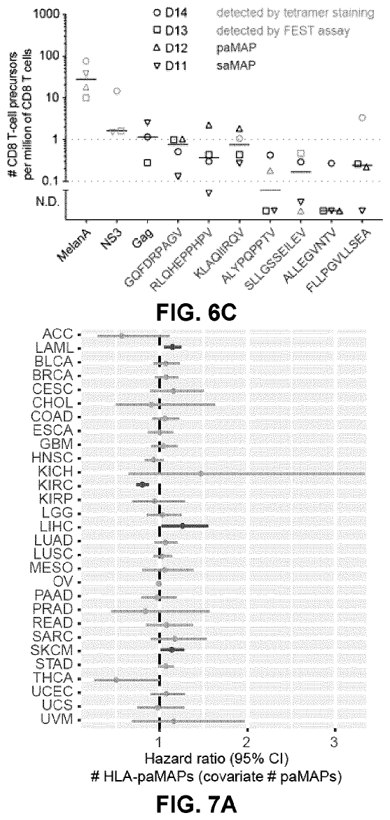

Significant T-cell responses against canonical paMAPs SLLGSSEILEV and KLAQIIRQV were detected by tetramer staining in one out of four donors (013 and 014, respectively) (FIG. 6A). The FEST assay also revealed the immunogenicity of four paMAPs in D12, for which no specific T-cell expansion was detected by tetramer staining (FIG. 6B). Following stimulation with a single peptide, a specific expansion of two to four TCRβ clonotypes against canonical paMAPs SLLGSSEILEV and LPMWKALLF, and the non-canonical paMAPs VTLSTYFHV and ALYPQPPTV (FIG. 6B, Tables 3A-B), was identified. Two additional TCRβ clonotypes were expanded following stimulation with a pool of HLA-A*02:01-binding paMAP (FIG. 6B, Tables 3A-B). Additionally, the immunogenicity of five saMAPs was assessed. It was found that, despite its expression in lymphoid precursor cells (FIG. 2B), the canonical saMAP FLLPGVLLSEA, deriving from the UDP glycosyltransferase family 3 member A2 (UGT3A2) gene, was immunogenic in one donor by tetramer staining (FIGS. 6A and B).

The stochasticity of paMAP and saMAP detection can be explained by low frequencies antigen-specific (i.e., tetramer⁺) CD8⁺ T-cells in donor PBMCs before in vitro stimulation, with a median of ≤0.75 paMAP-specific cells per 10⁶ CD8 T cells (FIG. 6C). Indeed, positive control peptides with high specific T-cell frequencies were consistently immunogenic by tetramer staining post-stimulation. In contrast, the positive control epitope Gag₇₇ (derived from the human immunodeficiency virus, HIV), which had specific T-cell frequencies similar to the paMAPs and saMAPs, was not immunogenic in any of the three PBMC donors tested (FIG. 6C). The low frequencies of Ag-specific T cells detected before in vitro priming suggest that they were in the naïve (rather than the memory) T-cell compartment.

In summary, five novel paMAPs (3/4 canonical and 2/7 non-canonical) and 1/5 canonical saMAPs were immunogenic in one or both T-cell assays (FIG. 12A). Their MCS was significantly more expressed in cancer samples than the corresponding normal tissues (FIGS. 2A, 3, 12B and 12C). These paMAPs had different origins: i) ZSCAN10, FOXH1, and TAF4, which are transcription factors (TFs) involved in pluripotency maintenance and embryonic development, and are known to promote self-renewal in cancer (Kazantseva et al., 2016; Loizou et al., 2019; Wang et al., 2019, 2007; Yu et al., 2009), ii) the oncofetal antigen CLDN6 (Reinhard et al., 2020), and iii) the prostate-cancer associated, “exonized” transposable element, PCAT14 (Babarinde et al., 2020; Prensner et al., 2011) (FIG. 12A). In addition, two of the paMAPs derived from MAGEA4 (GVYDGREHTV and KVLEHVVRV) and overexpressed in cancer samples (FIG. 12B) were immunogenic in previous studies (Duffour et al., 1999; Jia et al., 2010), altogether reinforcing the therapeutic potential of paMAPs.

### Example 8: paMAP and saMAP Expression Correlates with Immune Evasion

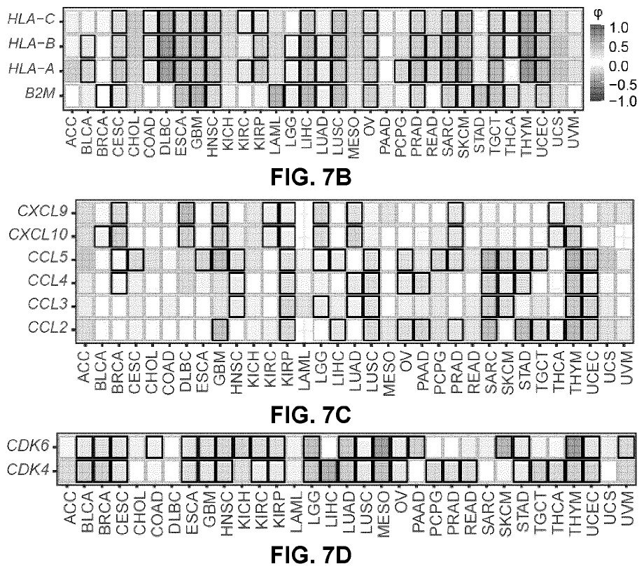

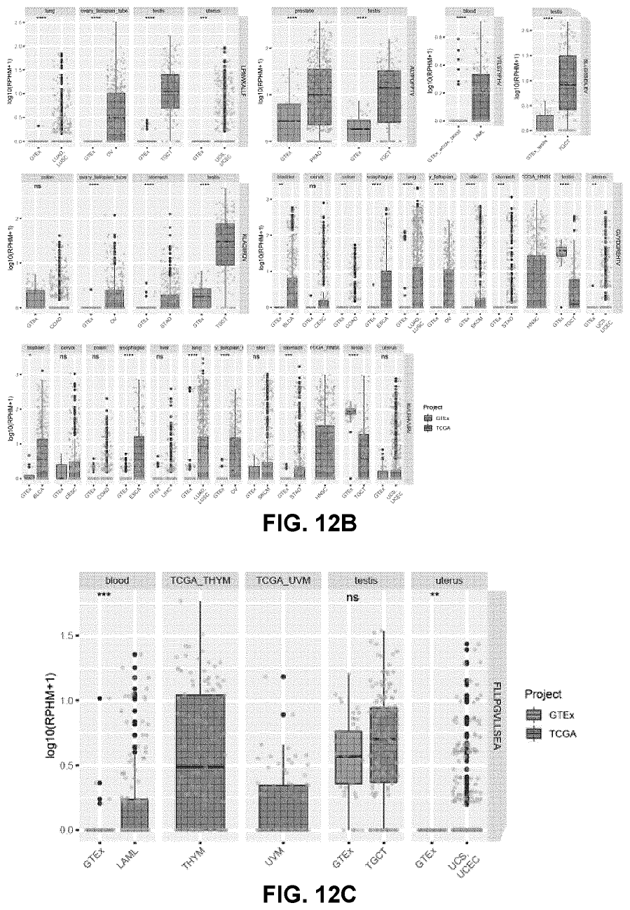

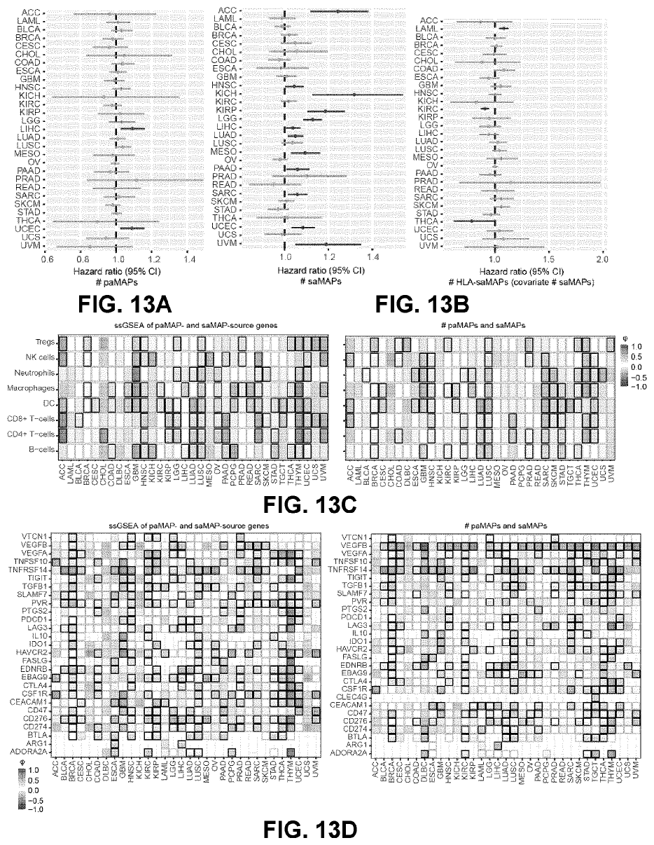

Having determined that paMAPs and saMAPs could be immunogenic, the effect of their HLA presentation on patient survival for the TCGA patient cohorts was evaluated using a Cox regression analysis. It was inferred that a paMAP or an saMAP was presented in a given sample if two conditions were met: expression of the MCS and presence of an HLA allotype that can bind and present the MAP according to the NetMHCpan-4.0 software (Jurtz et al., 2017) (Table 4). Hence, each MAP was assumed to be presented only in a fraction of the samples (bearing a relevant HLA allotype) in which its MCS was expressed. Presentation of paMAPs had an HLA-dependent positive impact on survival in renal clear cell carcinoma (KIRO), but either no or a minimally negative impact in other cancer types (FIGS. 7A and 13A). The same analysis performed using saMAPs showed similar results. The mere expression of saMAPs correlated in many cancer types with a shortened survival (FIG. 13A). However, presence of a relevant HLA allotype had a positive effect in KIRO and thyroid carcinoma (THCA), a negative effect in AML (LAML), and no effect in all other cancer types (FIG. 131B). Therefore, considering that inter-group differences were minimal, it may be concluded that the presentation of paMAPs and saMAPs did not confer a clear survival advantage in patients from the TOGA cohorts, which prompted us to investigate possible immune evasion mechanisms associated with their expression.

TABLE 4
HLA alleles from TCGA patient capable of binding paMAPs and saMAPs (promiscuous
binders), as calculated using NetMHCpan-4.0 (binding affinity rank < 2%). All TCGA patient
alleles were tested. HLA alleles from the iPSC samples studied were added to this list.

| MAP | HLA alleles |
|---|---|
| AKKKENITY | HLA-B15:03 HLA-B15:18 HLA-B15:46 |
| ALLEGVNTV | HLA-A02:01 HLA-C02:10 HLA-C04:01 HLA-A02:02 HLA-A02:06 |
| | HLA-A32:01 HLA-A02:05 HLA-A02:03 HLA-A02:07 HLA-A02:14 |
| | HLA-A02:17 HLA-A02:04 HLA-A69:01 HLA-A02:10 HLA-B13:02 |
| | HLA-B48:01 HLA-B13:01 HLA-B48:03 HLA-B15:30 HLA-B39:02 |
| | HLA-B15:58 HLA-C12:03 HLA-C01:02 HLA-C05:01 HLA-C02:02 |
| | HLA-C16:01 HLA-C07:04 HLA-C08:02 HLA-C04:07 HLA-C17:01 |
| | HLA-C16:02 HLA-C04:06 HLA-C08:01 HLA-C12:02 HLA-C08:04 |
| | HLA-C16:04 HLA-C04:03 HLA-C01:03 HLA-C04:04 HLA-C08:03 |
| ALLEGVNTVVV | HLA-A02:01 HLA-A02:02 HLA-A02:06 HLA-A02:03 HLA-A02:07 |
| | HLA-A02:14 HLA-A02:17 HLA-A02:04 HLA-A02:10 |
| ALYPQPPTV | HLA-A02:01 HLA-C03:04 HLA-C02:10 HLA-C04:01 HLA-A02:02 |
| | HLA-A02:06 HLA-A32:01 HLA-A02:05 HLA-A02:03 HLA-A02:07 |
| | HLA-A02:14 HLA-A02:17 HLA-A02:04 HLA-A02:10 HLA-B13:02 |
| | HLA-B52:01 HLA-B48:01 HLA-B46:01 HLA-B13:01 HLA-B48:03 |
| | HLA-B51:07 HLA-B15:30 HLA-B15:58 HLA-C06:02 HLA-C07:06 |
| | HLA-C07:02 HLA-C12:03 HLA-C07:01 HLA-C14:02 HLA-C01:02 |
| | HLA-C05:01 HLA-C03:03 HLA-C02:02 HLA-C16:01 HLA-C07:04 |
| | HLA-C08:02 HLA-C15:02 HLA-C07:18 HLA-C15:05 HLA-C04:07 |
| | HLA-C17:01 HLA-C03:02 HLA-C16:02 HLA-C04:06 HLA-C08:01 |
| | HLA-C12:02 HLA-C08:04 HLA-C16:04 HLA-C14:03 HLA-C04:03 |
| | HLA-C01:03 HLA-C04:04 HLA-C15:04 HLA-C08:03 HLA-C07:17 |
| | HLA-C07:05 |
| AMYHALEKA | HLA-A02:01 HLA-A02:02 HLA-A02:06 HLA-A02:05 HLA-A02:03 |
| | HLA-A02:07 HLA-A02:14 HLA-A02:17 HLA-A02:04 HLA-A02:10 |
| ASTPASSEL | HLA-C03:04 HLA-C02:10 HLA-B46:01 HLA-B57:03 HLA-B15:17 |
| | HLA-B15:16 HLA-B57:02 HLA-B15:30 HLA-B15:58 HLA-B57:04 |
| | HLA-C12:03 HLA-C01:02 HLA-C05:01 HLA-C03:03 HLA-C02:02 |
| | HLA-C16:01 HLA-C08:02 HLA-C15:02 HLA-C15:05 HLA-C17:01 |
| | HLA-C03:02 HLA-C16:02 HLA-C04:06 HLA-C08:01 HLA-C12:02 |
| | HLA-C08:04 HLA-C16:04 HLA-C04:03 |
| | HLA-C08:03 |
| EEEIHSPTL | HLA-B40:01 HLA-B18:01 HLA-B44:02 HLA-B40:02 HLA-B44:03 |
| | HLA-B37:01 HLA-B41:01 HLA-B45:01 HLA-B41:02 HLA-B44:05 |
| | HLA-B48:03 HLA-B14:03 HLA-B38:02 HLA-B44:15 HLA-B15:37 |
| | HLA-B18:03 HLA-B15:09 HLA-B44:10 HLA-B41:03 HLA-B39:09 |
| FAYPNQKDF | HLA-C03:04 HLA-C02:10 HLA-B15:03 HLA-B53:01 HLA-C04:01 |
| | HLA-A25:01 HLA-B52:01 HLA-B35:03 HLA-B35:01 HLA-B51:01 |
| | HLA-B15:18 HLA-B35:02 HLA-B58:01 HLA-B35:05 HLA-B46:01 |
| | HLA-B57:03 HLA-B56:04 HLA-B15:02 HLA-B58:02 HLA-B15:17 |
| | HLA-B51:02 HLA-B51:08 HLA-B15:10 HLA-B15:05 HLA-B51:07 |
| | HLA-B35:08 HLA-B15:25 HLA-B15:37 HLA-B59:01 HLA-B15:08 |
| | HLA-B15:16 HLA-B15:11 HLA-B78:01 HLA-B15:09 HLA-B15:13 |
| | HLA-B57:02 HLA-B15:20 HLA-B15:06 HLA-B15:31 HLA-B15:46 |
| | HLA-B57:04 HLA-C06:02 HLA-C07:06 HLA-C07:02 HLA-C12:03 |
| | HLA-C07:01 HLA-C14:02 HLA-C01:02 HLA-C05:01 HLA-C03:03 |
| | HLA-C02:02 HLA-C16:01 HLA-C07:04 HLA-C08:02 HLA-C15:02 |
| | HLA-C07:18 HLA-C15:05 HLA-C04:07 HLA-C17:01 HLA-C03:02 |
| | HLA-C16:02 HLA-C04:06 HLA-C08:01 HLA-C12:02 HLA-C08:04 |
| | HLA-C16:04 HLA-C14:03 HLA-C04:03 HLA-C01:03 HLA-C04:04 |
| | HLA-C15:04 HLA-C08:03 HLA-C07:17 HLA-C07:05 |
| FAYPNQKVTF | HLA-C03:04 HLA-C02:10 HLA-B15:03 HLA-B53:01 HLA-C04:01 |
| | HLA-A25:01 HLA-A24:03 HLA-A24:02 HLA-A23:01 HLA-A24:07 |
| | HLA-A24:10 HLA-A24:20 HLA-B52:01 HLA-B35:03 HLA-B35:01 |
| | HLA-B15:01 HLA-B51:01 HLA-B15:18 HLA-B35:02 HLA-B58:01 |
| | HLA-B35:05 HLA-B46:01 HLA-B39:10 HLA-B67:01 HLA-B57:01 |
| | HLA-B57:03 HLA-B56:04 HLA-B15:02 HLA-B58:02 HLA-B15:17 |
| | HLA-B51:02 HLA-B81:01 HLA-B51:08 HLA-B15:10 HLA-B15:05 |
| | HLA-B51:07 HLA-B35:08 HLA-B15:25 HLA-B15:37 HLA-B59:01 |
| | HLA-B15:08 HLA-B15:16 HLA-B15:07 HLA-B15:11 HLA-B78:01 |
| | HLA-B15:09 HLA-B55:04 HLA-B56:01 HLA-B15:12 HLA-B15:13 |
| | HLA-B57:02 HLA-B15:20 HLA-B15:30 HLA-B15:06 HLA-B15:27 |
| | HLA-B15:31 HLA-B15:46 HLA-B15:58 HLA-B57:04 HLA-C06:02 |
| | HLA-C07:06 HLA-C07:02 HLA-C12:03 HLA-C07:01 HLA-C14:02 |
| | HLA-C01:02 HLA-C05:01 HLA-C03:03 HLA-C02:02 HLA-C16:01 |
| | HLA-C07:04 HLA-C08:02 HLA-C15:02 HLA-C07:18 HLA-C15:05 |
| | HLA-C04:07 HLA-C17:01 HLA-C03:02 HLA-C16:02 HLA-C04:06 |
| | HLA-C08:01 HLA-C12:02 HLA-C08:04 HLA-C16:04 HLA-C14:03 |
| | HLA-C04:03 HLA-C01:03 HLA-C04:04 HLA-C15:04 HLA-C08:03 |
| | HLA-C07:17 HLA-C07:05 |
| FGDGKFSEV | HLA-C03:04 HLA-C02:10 HLA-C04:01 HLA-A02:06 HLA-A02:05 |
| | HLA-A02:07 HLA-A02:14 HLA-A02:10 HLA-B39:09 HLA-C06:02 |
| | HLA-C07:06 HLA-C12:03 HLA-C07:01 HLA-C14:02 HLA-C01:02 |
| | HLA-C05:01 HLA-C03:03 HLA-C02:02 HLA-C16:01 HLA-C07:04 |
| | HLA-C08:02 HLA-C15:02 HLA-C07:18 HLA-C15:05 HLA-C04:07 |
| | HLA-C17:01 HLA-C16:02 HLA-C04:06 HLA-C08:01 HLA-C12:02 |
| | HLA-C08:04 HLA-C16:04 HLA-C14:03 HLA-C04:03 HLA-C01:03 |
| | HLA-C04:04 HLA-C15:04 HLA-C08:03 HLA-C07:17 HLA-C07:05 |
| FHSQNSPIRY | HLA-C02:10 HLA-B15:03 HLA-A01:01 HLA-A30:02 HLA-A29:02 |
| | HLA-A01:03 HLA-A30:04 HLA-A29:01 HLA-A36:01 HLA-A01:02 |
| | HLA-A80:01 HLA-B15:18 HLA-B38:01 HLA-B15:10 HLA-B38:02 |
| | HLA-B15:37 HLA-B15:09 HLA-C07:06 HLA-C07:02 HLA-C07:01 |
| | HLA-C02:02 HLA-C07:18 HLA-C15:04 HLA-C07:17 |
| FLLPGVLLSEA | HLA-A02:01 HLA-A02:02 HLA-A02:06 HLA-A02:05 HLA-A02:03 |
| | HLA-A02:07 HLA-A02:14 HLA-A02:17 HLA-A02:04 HLA-A02:10 |
| | HLA-B55:02 HLA-B54:01 HLA-B56:01 |
| FTEEDLHFVLY | HLA-A01:01 HLA-A26:01 HLA-A29:02 HLA-A01:03 HLA-A29:01 |
| | HLA-A36:01 HLA-A26:08 HLA-A01:02 HLA-A80:01 HLA-A26:02 |
| | HLA-A43:01 HLA-B18:01 HLA-B18:03 HLA-C05:01 |
| GEHLVSVTL | HLA-B40:01 HLA-B13:02 HLA-B48:01 HLA-B18:01 HLA-B44:02 |
| | HLA-B40:02 HLA-B38:01 HLA-B44:03 HLA-B37:01 HLA-B41:01 |
| | HLA-B13:01 HLA-B49:01 HLA-B45:01 HLA-B41:02 HLA-B50:01 |
| | HLA-B44:05 HLA-B40:06 HLA-B48:03 HLA-B38:02 HLA-B44:15 |
| | HLA-B18:03 HLA-B47:01 HLA-B44:10 HLA-B39:02 HLA-B41:03 |
| GEIKTFSDL | HLA-B40:01 HLA-B48:01 HLA-B18:01 HLA-B44:02 HLA-B40:02 |
| | HLA-B44:03 HLA-B37:01 HLA-B41:01 HLA-B13:01 HLA-B49:01 |
| | HLA-B45:01 HLA-B41:02 HLA-B50:01 HLA-B44:05 HLA-B40:06 |
| | HLA-B48:03 HLA-B44:15 HLA-B18:03 HLA-B47:01 HLA-B44:10 |
| | HLA-B39:02 HLA-B15:46 HLA-B41:03 |
| GKFQGLIEKF | HLA-B15:03 HLA-A24:03 HLA-A24:02 HLA-A23:01 HLA-A24:07 |
| | HLA-A24:10 HLA-A24:20 HLA-B27:02 HLA-B47:01 HLA-B15:46 |
| GKLDNTNEY | HLA-B15:03 HLA-B15:18 HLA-B15:46 |
| GLYDGREHSV | HLA-A02:01 HLA-C04:01 HLA-A02:02 HLA-A02:06 HLA-A02:05 |
| | HLA-A02:03 HLA-A02:07 HLA-A02:14 HLA-A02:17 HLA-A02:04 |
| | HLA-A02:10 HLA-C04:07 |
| GQAHPQGSF | HLA-B15:03 HLA-B48:01 HLA-B15:01 HLA-B15:18 HLA-B13:01 |
| | HLA-B15:02 HLA-B48:03 HLA-B15:05 HLA-B15:25 HLA-B47:01 |
| | HLA-B15:07 HLA-B15:12 HLA-B15:13 HLA-B15:20 HLA-B15:30 |
| | HLA-B15:06 HLA-B15:27 HLA-B15:31 HLA-B15:46 HLA-B15:58 |
| GQFDRPAGV | HLA-A02:01 HLA-C02:10 HLA-A02:02 HLA-A02:06 HLA-A02:05 |
| | HLA-A02:03 HLA-A02:07 HLA-A02:14 HLA-A02:17 HLA-A02:04 |
| | HLA-A02:10 HLA-B13:02 HLA-B52:01 HLA-B48:01 HLA-B37:01 |
| | HLA-B13:01 HLA-B49:01 HLA-B48:03 HLA-B27:06 HLA-B47:01 |
| | HLA-B15:30 HLA-B39:02 HLA-B15:58 HLA-C06:02 HLA-C07:06 |
| | HLA-C07:01 HLA-C02:02 HLA-C07:04 HLA-C07:18 HLA-C17:01 |
| | HLA-C04:04 HLA-C07:05 |
| GQFLVKSGY | HLA-B15:03 HLA-A30:02 HLA-A29:02 HLA-A30:04 HLA-A29:01 |
| | HLA-A80:01 HLA-B18:01 HLA-B15:01 HLA-B15:18 HLA-B27:02 |
| | HLA-B15:02 HLA-B15:05 HLA-B15:25 HLA-B18:03 HLA-B47:01 |
| | HLA-B15:07 HLA-B15:12 HLA-B15:20 HLA-B15:06 HLA-B15:27 |
| | HLA-B15:31 HLA-B15:46 |
| GQITHNTSF | HLA-B40:01 HLA-B15:03 HLA-B48:01 HLA-B18:01 HLA-B15:01 |
| | HLA-B40:02 HLA-B15:18 HLA-B38:01 HLA-B37:01 HLA-B27:02 |
| | HLA-B46:01 HLA-B13:01 HLA-B50:01 HLA-B15:02 HLA-B48:03 |
| | HLA-B27:06 HLA-B15:10 HLA-B15:05 HLA-B38:02 HLA-B15:25 |
| | HLA-B15:37 HLA-B15:08 HLA-B27:04 HLA-B47:01 HLA-B15:07 |
| | HLA-B15:11 HLA-B15:09 HLA-B15:12 HLA-B15:13 HLA-B15:20 |
| | HLA-B15:30 HLA-B15:06 HLA-B15:27 HLA-B39:02 HLA-B15:31 |
| | HLA-B15:46 HLA-B15:58 HLA-B41:03 HLA-C03:02 HLA-C12:02 |
| GQKENVVVY | HLA-B15:03 HLA-A30:02 HLA-A30:04 HLA-B18:01 HLA-B15:01 |
| | HLA-B15:18 HLA-B46:01 HLA-B15:02 HLA-B15:05 HLA-B15:25 |
| | HLA-B15:08 HLA-B18:03 HLA-B47:01 HLA-B15:07 HLA-B15:11 |
| | HLA-B15:12 HLA-B15:20 HLA-B15:30 HLA-B15:06 HLA-B15:27 |
| | HLA-B15:31 HLA-B15:46 |
| GVYDGREHTV | HLA-C04:01 HLA-A02:06 HLA-A02:05 HLA-A02:03 HLA-C04:07 |
| HPLPGLILEW | HLA-B53:01 HLA-B35:03 HLA-B35:01 HLA-B51:01 HLA-B35:02 |
| | HLA-B58:01 HLA-B35:05 HLA-B57:01 HLA-B57:03 HLA-B56:04 |
| | HLA-B58:02 HLA-B51:02 HLA-B51:08 HLA-B35:08 HLA-B59:01 |
| | HLA-B82:01 HLA-B15:13 HLA-B57:02 HLA-B82:02 HLA-B57:04 |
| IPMKIYLW | HLA-B53:01 HLA-B51:01 HLA-B51:02 HLA-B51:08 HLA-B42:02 |
| | HLA-B51:07 HLA-B59:01 |
| ISMCDLVY | HLA-A01:01 HLA-A30:02 HLA-A29:02 HLA-A01:03 HLA-A29:01 |
| | HLA-A36:01 HLA-A01:02 HLA-A80:01 HLA-B35:01 HLA-B35:05 |
| | HLA-B15:02 HLA-B15:17 HLA-B15:05 HLA-B35:08 HLA-B15:25 |
| | HLA-B15:16 HLA-B15:20 HLA-B15:06 HLA-B57:04 |
| KHFDSPRGVAF | HLA-B15:03 HLA-C04:01 HLA-B15:18 HLA-B38:01 HLA-B15:10 |
| | HLA-B38:02 HLA-B15:37 HLA-B15:09 HLA-C07:02 HLA-C14:02 |
| | HLA-C04:07 HLA-C14:03 HLA-C07:17 |
| KLAQIIRQV | HLA-A02:01 HLA-C02:10 HLA-A02:02 HLA-A02:06 HLA-A32:01 |
| | HLA-A02:05 HLA-A02:03 HLA-A02:07 HLA-A02:14 HLA-A02:17 |
| | HLA-A02:04 HLA-A02:10 HLA-C02:02 HLA-C15:02 HLA-C17:01 |
| | HLA-C16:02 |
| KLDSYIIPY | HLA-B15:03 HLA-A01:01 HLA-C04:01 HLA-A03:01 HLA-A11:01 |
| | HLA-A30:02 HLA-A29:02 HLA-A01:03 HLA-A03:02 HLA-A30:04 |
| | HLA-A32:01 HLA-A29:01 HLA-A36:01 HLA-A74:01 HLA-A11:02 |
| | HLA-A01:02 HLA-A74:03 HLA-A11:12 HLA-A80:01 HLA-A11:03 |
| | HLA-B35:01 HLA-B15:01 HLA-B15:18 HLA-B15:02 HLA-B15:05 |
| | HLA-B35:08 HLA-B15:25 HLA-B15:12 HLA-B15:20 HLA-B15:06 |
| | HLA-B15:27 HLA-B15:31 HLA-C05:01 HLA-C08:02 HLA-C04:07 |
| | HLA-C16:02 HLA-C04:06 HLA-C04:03 HLA-C15:04 |
| KLQEKIQEL | HLA-A02:01 HLA-C02:10 HLA-C04:01 HLA-A02:02 HLA-A02:06 |
| | HLA-A32:01 HLA-A02:05 HLA-A02:03 HLA-A02:07 HLA-A02:14 |
| | HLA-A02:17 HLA-A02:04 HLA-A02:10 HLA-B48:01 HLA-B08:01 |
| | HLA-B42:01 HLA-B48:03 HLA-B27:06 HLA-B42:02 HLA-B14:03 |
| | HLA-B15:30 HLA-B39:02 HLA-B15:58 HLA-C06:02 HLA-C07:06 |
| | HLA-C07:02 HLA-C07:01 HLA-C14:02 HLA-C01:02 HLA-C05:01 |
| | HLA-C02:02 HLA-C16:01 HLA-C07:04 HLA-C08:02 HLA-C07:18 |
| | HLA-C04:07 HLA-C17:01 HLA-C16:02 HLA-C04:06 HLA-C08:04 |
| | HLA-C14:03 HLA-C04:03 HLA-C01:03 HLA-C04:04 HLA-C07:17 |
| | HLA-C07:05 |
| KMSELQTYV | HLA-A02:01 HLA-C02:10 HLA-C04:01 HLA-A02:02 HLA-A30:01 |
| | HLA-A30:04 HLA-A02:06 HLA-A32:01 HLA-A02:05 HLA-A74:01 |
| | HLA-A02:03 HLA-A02:07 HLA-A02:14 HLA-A02:17 HLA-A02:04 |
| | HLA-A74:03 HLA-A02:10 HLA-B13:02 HLA-B48:01 HLA-B46:01 |
| | HLA-B13:01 HLA-B48:03 HLA-B15:30 HLA-B39:02 HLA-B15:58 |
| | HLA-C06:02 HLA-C07:06 HLA-C07:02 HLA-C12:03 HLA-C07:01 |
| | HLA-C01:02 HLA-C05:01 HLA-C02:02 HLA-C16:01 HLA-C07:04 |
| | HLA-C08:02 HLA-C15:02 HLA-C07:18 HLA-C15:05 HLA-C04:07 |
| | HLA-C17:01 HLA-C16:02 HLA-C04:06 HLA-C08:01 HLA-C12:02 |
| | HLA-C08:04 HLA-C16:04 HLA-C04:03 HLA-C01:03 HLA-C04:04 |
| | HLA-C15:04 HLA-C08:03 HLA-C07:05 |
| KQFEGTVEI | HLA-A02:01 HLA-B40:01 HLA-C03:04 HLA-C02:10 HLA-B15:03 |
| | HLA-C04:01 HLA-A02:02 HLA-A30:04 HLA-A02:06 HLA-A32:01 |
| | HLA-A02:05 HLA-A24:10 HLA-A02:03 HLA-A02:07 HLA-A02:14 |
| | HLA-A02:17 HLA-A02:04 HLA-A02:10 HLA-B13:02 HLA-B52:01 |
| | HLA-B48:01 HLA-B15:01 HLA-B40:02 HLA-B15:18 HLA-B38:01 |
| | HLA-B39:01 HLA-B37:01 HLA-B27:02 HLA-B41:01 HLA-B46:01 |
| | HLA-B13:01 HLA-B14:02 HLA-B49:01 HLA-B39:06 HLA-B41:02 |
| | HLA-B50:01 HLA-B40:06 HLA-B48:03 HLA-B27:06 HLA-B15:10 |
| | HLA-B15:05 HLA-B27:03 HLA-B38:02 HLA-B51:07 HLA-B15:25 |
| | HLA-B15:37 HLA-B27:04 HLA-B47:01 HLA-B15:07 HLA-B15:09 |
| | HLA-B15:12 HLA-B15:13 HLA-B73:01 HLA-B15:20 HLA-B15:30 |
| | HLA-B15:06 HLA-B15:27 HLA-B44:10 HLA-B39:02 HLA-B15:46 |
| | HLA-B15:58 HLA-B41:03 HLA-B39:09 HLA-C06:02 HLA-C07:06 |
| | HLA-C07:02 HLA-C12:03 HLA-C07:01 HLA-C14:02 HLA-C01:02 |
| | HLA-C05:01 HLA-C03:03 HLA-C02:02 HLA-C16:01 HLA-C07:04 |
| | HLA-C08:02 HLA-C15:02 HLA-C07:18 HLA-C15:05 HLA-C04:07 |
| | HLA-C17:01 HLA-C03:02 HLA-C16:02 HLA-C04:06 HLA-C08:01 |
| | HLA-C12:02 HLA-C08:04 HLA-C16:04 HLA-C14:03 HLA-C04:03 |
| | HLA-C01:03 HLA-C04:04 HLA-C15:04 HLA-C08:03 HLA-C07:17 |
| | HLA-C07:05 |
| KQMENDIQLY | HLA-B15:03 HLA-A01:01 HLA-A30:02 HLA-A29:02 HLA-A01:03 |
| | HLA-A30:04 HLA-A32:01 HLA-A29:01 HLA-A36:01 HLA-A01:02 |
| | HLA-A80:01 HLA-B48:01 HLA-B15:01 HLA-B44:02 HLA-B15:18 |
| | HLA-B44:03 HLA-B27:02 HLA-B13:01 HLA-B15:02 HLA-B48:03 |
| | HLA-B15:05 HLA-B15:25 HLA-B27:04 HLA-B47:01 HLA-B15:07 |
| | HLA-B15:12 HLA-B15:13 HLA-B15:20 HLA-B15:30 HLA-B15:06 |
| | HLA-B15:27 HLA-B39:02 HLA-B15:31 HLA-B15:46 HLA-B15:58 |
| KVLEHVVRV | HLA-A02:01 HLA-C02:10 HLA-A02:02 HLA-A30:01 HLA-A02:06 |
| | HLA-A32:01 HLA-A02:05 HLA-A02:03 HLA-A02:07 HLA-A02:14 |
| | HLA-A02:17 HLA-A02:04 HLA-A69:01 HLA-A02:10 HLA-B13:02 |
| | HLA-B52:01 HLA-C06:02 HLA-C07:06 HLA-C07:02 HLA-C12:03 |
| | HLA-C07:01 HLA-C01:02 HLA-C05:01 HLA-C02:02 HLA-C16:01 |
| | HLA-C07:04 HLA-C15:02 HLA-C07:18 HLA-C15:05 HLA-C17:01 |
| | HLA-C16:02 HLA-C04:06 HLA-C08:01 HLA-C12:02 HLA-C08:04 |
| | HLA-C16:04 HLA-C04:03 HLA-C01:03 HLA-C04:04 HLA-C15:04 |
| | HLA-C08:03 HLA-C07:17 HLA-C07:05 |
| LALGNTKEL | HLA-C03:04 HLA-C02:10 HLA-B53:01 HLA-B35:03 HLA-B35:01 |
| | HLA-B51:01 HLA-B15:18 HLA-B35:02 HLA-B35:05 HLA-B46:01 |
| | HLA-B39:10 HLA-B67:01 HLA-B56:04 HLA-B51:02 HLA-B81:01 |
| | HLA-B51:08 HLA-B15:10 HLA-B35:08 HLA-B15:37 HLA-B15:08 |
| | HLA-B78:01 HLA-B15:09 HLA-B55:04 HLA-C12:03 HLA-C14:02 |
| | HLA-C01:02 HLA-C03:03 HLA-C02:02 HLA-C16:01 HLA-C08:02 |
| | HLA-C15:02 HLA-C15:05 HLA-C17:01 HLA-C03:02 HLA-C16:02 |
| | HLA-C04:06 HLA-C08:01 HLA-C12:02 HLA-C08:04 HLA-C16:04 |
| | HLA-C14:03 HLA-C01:03 HLA-C15:04 HLA-C08:03 HLA-C07:17 |
| LPMWKALLF | HLA-B07:02 HLA-C03:04 HLA-B53:01 HLA-C04:01 HLA-A25:01 |
| | HLA-A29:02 HLA-A29:01 HLA-A24:07 HLA-B52:01 HLA-B35:03 |
| | HLA-B08:01 HLA-B35:01 HLA-B18:01 HLA-B55:01 HLA-B51:01 |
| | HLA-B15:18 HLA-B35:02 HLA-B38:01 HLA-B39:01 HLA-B07:05 |
| | HLA-B55:02 HLA-B35:05 HLA-B54:01 HLA-B42:01 HLA-B39:10 |
| | HLA-B14:02 HLA-B67:01 HLA-B56:04 HLA-B15:02 HLA-B51:02 |
| | HLA-B81:01 HLA-B51:08 HLA-B15:10 HLA-B42:02 HLA-B14:03 |
| | HLA-B38:02 HLA-B51:07 HLA-B35:08 HLA-B15:37 HLA-B59:01 |
| | HLA-B15:08 HLA-B18:03 HLA-B15:11 HLA-B78:01 HLA-B15:09 |
| | HLA-B82:01 HLA-B55:04 HLA-B56:01 HLA-B15:13 HLA-B07:04 |
| | HLA-B82:02 HLA-B15:31 HLA-B39:09 HLA-C12:03 HLA-C14:02 |
| | HLA-C01:02 HLA-C03:03 HLA-C16:01 HLA-C08:02 HLA-C04:07 |
| | HLA-C03:02 HLA-C16:02 HLA-C04:06 HLA-C08:01 HLA-C08:04 |
| | HLA-C16:04 HLA-C14:03 HLA-C01:03 HLA-C04:04 HLA-C08:03 |
| | HLA-C07:17 |
| LPSGETIAKW | HLA-B53:01 HLA-B35:03 HLA-B35:01 HLA-B58:01 HLA-B35:05 |
| | HLA-B57:01 HLA-B57:03 HLA-B58:02 HLA-B15:17 HLA-B51:08 |
| | HLA-B35:08 HLA-B59:01 HLA-B15:16 HLA-B15:13 HLA-B57:02 |
| | HLA-B57:04 |
| MAWNGILHL | HLA-A02:01 HLA-C03:04 HLA-C02:10 HLA-B15:03 HLA-B53:01 |
| | HLA-C04:01 HLA-A68:02 HLA-A29:02 HLA-A02:06 HLA-A32:01 |
| | HLA-A29:01 HLA-A02:05 HLA-A02:07 HLA-A02:14 HLA-A02:17 |
| | HLA-A02:04 HLA-A69:01 HLA-A02:10 HLA-B13:02 HLA-B52:01 |
| | HLA-B35:03 HLA-B48:01 HLA-B35:01 HLA-B15:01 HLA-B55:01 |
| | HLA-B51:01 HLA-B15:18 HLA-B35:02 HLA-B38:01 HLA-B39:01 |
| | HLA-B58:01 HLA-B55:02 HLA-B37:01 HLA-B35:05 HLA-B54:01 |
| | HLA-B42:01 HLA-B46:01 HLA-B39:10 HLA-B13:01 HLA-B14:02 |
| | HLA-B67:01 HLA-B49:01 HLA-B57:01 HLA-B57:03 HLA-B39:06 |
| | HLA-B56:04 HLA-B15:02 HLA-B58:02 HLA-B15:17 HLA-B51:02 |
| | HLA-B81:01 HLA-B51:08 HLA-B48:03 HLA-B27:06 HLA-B15:10 |
| | HLA-B15:05 HLA-B42:02 HLA-B14:03 HLA-B38:02 HLA-B51:07 |
| | HLA-B35:08 HLA-B15:25 HLA-B15:37 HLA-B59:01 HLA-B15:08 |
| | HLA-B15:16 HLA-B18:03 HLA-B47:01 HLA-B15:07 HLA-B15:11 |
| | HLA-B78:01 HLA-B15:09 HLA-B82:01 HLA-B55:04 HLA-B56:01 |
| | HLA-B15:12 HLA-B15:13 HLA-B73:01 HLA-B57:02 HLA-B07:04 |
| | HLA-B15:20 HLA-B15:30 HLA-B15:06 HLA-B82:02 HLA-B39:02 |
| | HLA-B15:31 HLA-B15:46 HLA-B15:58 HLA-B39:09 HLA-B57:04 |
| | HLA-C06:02 HLA-C07:06 HLA-C07:02 HLA-C12:03 HLA-C07:01 |
| | HLA-C14:02 HLA-C01:02 HLA-C05:01 HLA-C03:03 HLA-C02:02 |
| | HLA-C16:01 HLA-C07:04 HLA-C08:02 HLA-C15:02 HLA-C07:18 |
| | HLA-C15:05 HLA-C04:07 HLA-C17:01 HLA-C03:02 HLA-C16:02 |
| | HLA-C04:06 HLA-C08:01 HLA-C12:02 HLA-C08:04 HLA-C16:04 |
| | HLA-C14:03 HLA-C04:03 HLA-C01:03 HLA-C04:04 HLA-C15:04 |
| | HLA-C08:03 HLA-C07:17 HLA-C07:05 |
| MKFGNQVSGLF | HLA-B15:03 HLA-B15:18 HLA-B38:01 HLA-B27:02 HLA-B15:10 |
| | HLA-B38:02 HLA-B47:01 HLA-B15:46 |
| NPIGDTGVKF | HLA-B07:02 HLA-B53:01 HLA-B35:03 HLA-B35:01 HLA-B35:02 |
| | HLA-B07:05 HLA-B35:05 HLA-B42:01 HLA-B39:10 HLA-B67:01 |
| | HLA-B56:04 HLA-B81:01 HLA-B35:08 HLA-B15:08 HLA-B15:11 |
| | HLA-B82:01 HLA-B55:04 HLA-B82:02 |
| NTENYILWGY | HLA-A01:01 HLA-A68:01 HLA-A25:01 HLA-A26:01 HLA-A30:02 |
| | HLA-A29:02 HLA-A01:03 HLA-A30:04 HLA-A29:01 HLA-A11:10 |
| | HLA-A36:01 HLA-A66:02 HLA-A66:01 HLA-A34:01 HLA-A26:08 |
| | HLA-A01:02 HLA-A80:01 HLA-A66:03 HLA-A26:03 HLA-A26:02 |
| | HLA-A43:01 HLA-B18:01 HLA-B44:02 HLA-B44:03 HLA-B44:05 |
| | HLA-B18:03 HLA-B47:01 HLA-B44:10 |
| NTLSESYIY | HLA-A01:01 HLA-B53:01 HLA-A25:01 HLA-A26:01 HLA-A30:02 |
| | HLA-A29:02 HLA-A01:03 HLA-A30:04 HLA-A29:01 HLA-A11:10 |
| | HLA-A36:01 HLA-A26:08 HLA-A01:02 HLA-A34:02 HLA-A80:01 |
| | HLA-A26:03 HLA-A26:02 HLA-A43:01 HLA-B35:01 HLA-B35:05 |
| | HLA-B15:02 HLA-B15:05 HLA-B35:08 HLA-B15:08 HLA-B15:11 |
| | HLA-B15:13 HLA-B15:20 HLA-B15:31 HLA-B57:04 |
| QPLPEPLQLW | HLA-B53:01 HLA-B58:02 HLA-B51:08 HLA-B15:13 HLA-B57:02 |
| | HLA-B57:04 |
| QPLPQPLELW | HLA-B53:01 HLA-B58:02 HLA-B51:08 HLA-B15:13 HLA-B57:02 |
| | HLA-B57:04 |
| QTELNNSKQEY | HLA-A01:01 HLA-A01:03 HLA-A36:01 HLA-A01:02 |
| RLQHEPPHPV | HLA-A02:01 HLA-A02:02 HLA-A02:03 HLA-B13:02 HLA-B48:01 |
| | HLA-B48:03 HLA-B27:06 HLA-B15:30 HLA-B39:02 HLA-B15:58 |
| RLWNETVEL | HLA-A02:01 HLA-C03:04 HLA-C02:10 HLA-B15:03 HLA-C04:01 |
| | HLA-A02:02 HLA-A02:06 HLA-A32:01 HLA-A02:05 HLA-A74:01 |
| | HLA-A02:03 HLA-A02:07 HLA-A02:14 HLA-A02:17 HLA-A02:04 |
| | HLA-A74:03 HLA-A80:01 HLA-A02:10 HLA-B13:02 HLA-B48:01 |
| | HLA-B39:01 HLA-B13:01 HLA-B81:01 HLA-B48:03 HLA-B27:06 |
| | HLA-B15:05 HLA-B15:25 HLA-B27:04 HLA-B47:01 HLA-B15:20 |
| | HLA-B15:30 HLA-B15:06 HLA-B39:02 HLA-B15:58 HLA-B39:09 |
| | HLA-C07:02 HLA-C14:02 HLA-C01:02 HLA-C05:01 HLA-C03:03 |
| | HLA-C02:02 HLA-C16:01 HLA-C08:02 HLA-C15:02 HLA-C15:05 |
| | HLA-C04:07 HLA-C17:01 HLA-C03:02 HLA-C16:02 HLA-C04:06 |
| | HLA-C08:01 HLA-C08:04 HLA-C16:04 HLA-C14:03 HLA-C04:03 |
| | HLA-C01:03 HLA-C04:04 HLA-C15:04 HLA-C08:03 HLA-C07:17 |
| | HLA-C07:05 |
| RLWNETVELF | HLA-A02:01 HLA-B15:03 HLA-C04:01 HLA-A24:03 HLA-A30:02 |
| | HLA-A24:02 HLA-A23:01 HLA-A30:04 HLA-A32:01 HLA-A24:07 |
| | HLA-A24:10 HLA-A02:07 HLA-A02:17 HLA-A02:04 HLA-A24:20 |
| | HLA-A80:01 HLA-B13:02 HLA-B48:01 HLA-B15:01 HLA-B27:02 |
| | HLA-B13:01 HLA-B58:02 HLA-B15:05 HLA-B15:25 HLA-B47:01 |
| | HLA-B15:07 HLA-B15:12 HLA-B15:13 HLA-B15:20 HLA-B15:30 |
| | HLA-B15:06 HLA-B15:27 HLA-B15:46 HLA-C04:07 HLA-C04:04 |
| RPARPPAGL | HLA-B07:02 HLA-C03:04 HLA-B35:03 HLA-B55:01 HLA-B35:02 |
| | HLA-B07:05 HLA-B42:01 HLA-B39:10 HLA-B67:01 HLA-B56:04 |
| | HLA-B81:01 HLA-B15:10 HLA-B42:02 HLA-B14:03 HLA-B15:09 |
| | HLA-B82:01 HLA-B55:04 HLA-B07:04 HLA-B82:02 HLA-C01:02 |
| | HLA-C03:03 HLA-C08:02 HLA-C08:04 HLA-C01:03 |
| RTDTGKRVLY | HLA-A01:01 HLA-A30:02 HLA-A29:02 HLA-A01:03 HLA-A30:04 |
| | HLA-A29:01 HLA-A36:01 HLA-A01:02 HLA-A80:01 HLA-A11:03 |
| | HLA-B57:01 HLA-B15:17 HLA-B57:04 HLA-C05:01 HLA-C16:02 |
| | HLA-C04:03 HLA-C15:04 |
| SANVSKVSF | HLA-C03:04 HLA-C02:10 HLA-B15:03 HLA-B53:01 HLA-B35:03 |
| | HLA-B35:01 HLA-B15:01 HLA-B15:18 HLA-B35:02 HLA-B58:01 |
| | HLA-B35:05 HLA-B42:01 HLA-B46:01 HLA-B67:01 HLA-B57:01 |
| | HLA-B57:03 HLA-B56:04 HLA-B15:02 HLA-B58:02 HLA-B15:17 |
| | HLA-B81:01 HLA-B15:05 HLA-B42:02 HLA-B35:08 HLA-B15:25 |
| | HLA-B15:08 HLA-B15:07 HLA-B15:11 HLA-B82:01 HLA-B15:12 |
| | HLA-B15:13 HLA-B57:02 HLA-B15:20 HLA-B15:06 HLA-B15:27 |
| | HLA-B82:02 HLA-B15:31 HLA-B15:46 HLA-B57:04 HLA-C12:03 |
| | HLA-C14:02 HLA-C01:02 HLA-C03:03 HLA-C02:02 HLA-C16:01 |
| | HLA-C08:02 HLA-C03:02 HLA-C16:02 HLA-C08:01 HLA-C12:02 |
| | HLA-C08:04 HLA-C16:04 HLA-C14:03 HLA-C01:03 HLA-C15:04 |
| | HLA-C08:03 HLA-C07:17 |
| SAQGKPTYF | HLA-C03:04 HLA-C02:10 HLA-C04:01 HLA-B46:01 HLA-B15:02 |
| | HLA-B15:08 HLA-B15:11 HLA-B15:13 HLA-B57:02 HLA-C06:02 |
| | HLA-C07:06 HLA-C07:02 HLA-C12:03 HLA-C07:01 HLA-C14:02 |
| | HLA-C01:02 HLA-C03:03 HLA-C02:02 HLA-C16:01 HLA-C07:04 |
| | HLA-C08:02 HLA-C07:18 HLA-C04:07 HLA-C03:02 HLA-C16:02 |
| | HLA-C04:06 HLA-C08:01 HLA-C12:02 HLA-C08:04 HLA-C16:04 |
| | HLA-C14:03 HLA-C04:03 HLA-C01:03 HLA-C04:04 HLA-C15:04 |
| | HLA-C08:03 HLA-C07:17 HLA-C07:05 |
| SETHPPEVAL | HLA-B40:01 HLA-B48:01 HLA-B44:02 HLA-B40:02 HLA-B44:03 |
| | HLA-B37:01 HLA-B41:01 HLA-B41:02 HLA-B44:05 HLA-B40:06 |
| | HLA-B48:03 HLA-B44:15 HLA-B44:10 HLA-B39:02 HLA-B41:03 |
| | HLA-C15:05 |
| SEVSADKLVAL | HLA-B40:01 HLA-B48:01 HLA-B44:02 HLA-B40:02 HLA-B44:03 |
| | HLA-B37:01 HLA-B41:01 HLA-B13:01 HLA-B41:02 HLA-B50:01 |
| | HLA-B44:05 HLA-B40:06 HLA-B48:03 HLA-B44:15 HLA-B47:01 |
| | HLA-B44:10 HLA-B39:02 HLA-B41:03 |
| SKLRSTGQSF | HLA-B15:03 HLA-B15:01 HLA-B15:18 HLA-B46:01 HLA-B15:02 |
| | HLA-B15:05 HLA-B42:02 HLA-B15:25 HLA-B15:08 HLA-B15:07 |
| | HLA-B15:11 HLA-B15:12 HLA-B15:13 HLA-B07:04 HLA-B15:20 |
| | HLA-B15:30 HLA-B15:06 HLA-B15:27 HLA-B15:46 HLA-B15:58 |
| SLLGSSEILEV | HLA-A02:01 HLA-A02:02 HLA-A02:06 HLA-A02:03 HLA-A02:07 |
| | HLA-A02:14 HLA-A02:17 HLA-A02:04 HLA-A02:10 |
| SPQEASGVRW | HLA-B53:01 HLA-B44:02 HLA-B44:03 HLA-B35:08 HLA-B82:01 |
| | HLA-B15:13 HLA-B82:02 |
| SQNSPIRY | HLA-B15:03 HLA-A30:02 HLA-B15:01 HLA-B15:46 |
| SQSSLMLYL | HLA-A02:01 HLA-B40:01 HLA-C03:04 HLA-C02:10 HLA-B15:03 |
| | HLA-C04:01 HLA-A02:02 HLA-A30:04 HLA-A02:06 HLA-A32:01 |
| | HLA-A02:05 HLA-A02:03 HLA-A02:07 HLA-A02:14 HLA-A02:17 |
| | HLA-A02:04 HLA-A02:10 HLA-B13:02 HLA-B52:01 HLA-B48:01 |
| | HLA-B15:01 HLA-B40:02 HLA-B15:18 HLA-B38:01 HLA-B39:01 |
| | HLA-B37:01 HLA-B27:02 HLA-B46:01 HLA-B13:01 HLA-B14:02 |
| | HLA-B49:01 HLA-B39:06 HLA-B41:02 HLA-B50:01 HLA-B44:05 |
| | HLA-B48:03 HLA-B27:06 HLA-B15:10 HLA-B15:05 HLA-B14:03 |
| | HLA-B38:02 HLA-B44:15 HLA-B15:25 HLA-B15:37 HLA-B47:01 |
| | HLA-B15:07 HLA-B15:09 HLA-B15:12 HLA-B15:13 HLA-B15:20 |
| | HLA-B15:30 HLA-B15:06 HLA-B15:27 HLA-B44:10 HLA-B39:02 |
| | HLA-B15:31 HLA-B15:46 HLA-B15:58 HLA-B41:03 HLA-B39:09 |
| | HLA-C06:02 HLA-C07:06 HLA-C07:02 HLA-C12:03 HLA-C07:01 |
| | HLA-C14:02 HLA-C01:02 HLA-C05:01 HLA-C03:03 HLA-C02:02 |
| | HLA-C16:01 HLA-C07:04 HLA-C08:02 HLA-C15:02 HLA-C07:18 |
| | HLA-C15:05 HLA-C04:07 HLA-C17:01 HLA-C03:02 HLA-C16:02 |
| | HLA-C04:06 HLA-C08:01 HLA-C12:02 HLA-C08:04 HLA-C16:04 |
| | HLA-C14:03 HLA-C04:03 HLA-C01:03 HLA-C04:04 HLA-C15:04 |
| | HLA-C08:03 HLA-C07:17 HLA-C07:05 |
| TASDLNLKV | HLA-C03:04 HLA-C02:10 HLA-B53:01 HLA-A68:02 HLA-A69:01 |
| | HLA-B51:01 HLA-B51:02 HLA-B51:08 HLA-B51:07 HLA-B59:01 |
| | HLA-C12:03 HLA-C05:01 HLA-C03:03 HLA-C02:02 HLA-C08:02 |
| | HLA-C15:02 HLA-C15:05 HLA-C17:01 HLA-C16:02 HLA-C04:06 |
| | HLA-C08:01 HLA-C12:02 HLA-C08:04 HLA-C16:04 HLA-C04:03 |
| | HLA-C15:04 HLA-C08:03 |
| TEFQQIINL | HLA-B40:01 HLA-B13:02 HLA-B52:01 HLA-B48:01 HLA-B18:01 |
| | HLA-B44:02 HLA-B40:02 HLA-B38:01 HLA-B44:03 HLA-B39:01 |
| | HLA-B37:01 HLA-B41:01 HLA-B13:01 HLA-B49:01 HLA-B45:01 |
| | HLA-B41:02 HLA-B50:01 HLA-B44:05 HLA-B40:06 HLA-B48:03 |
| | HLA-B15:10 HLA-B38:02 HLA-B51:07 HLA-B44:15 HLA-B15:37 |
| | HLA-B18:03 HLA-B47:01 HLA-B15:09 HLA-B73:01 HLA-B44:10 |
| | HLA-B39:02 HLA-B41:03 HLA-B39:09 |
| TPSPIIQQL | HLA-B07:02 HLA-C03:04 HLA-B53:01 HLA-B35:03 HLA-B35:01 |
| | HLA-B55:01 HLA-B51:01 HLA-B35:02 HLA-B39:01 HLA-B07:05 |
| | HLA-B55:02 HLA-B35:05 HLA-B42:01 HLA-B39:10 HLA-B67:01 |
| | HLA-B56:04 HLA-B51:02 HLA-B81:01 HLA-B51:08 HLA-B15:10 |
| | HLA-B42:02 HLA-B14:03 HLA-B35:08 HLA-B15:37 HLA-B59:01 |
| | HLA-B78:01 HLA-B15:09 HLA-B82:01 HLA-B55:04 HLA-B56:01 |
| | HLA-B07:04 HLA-B82:02 HLA-C01:02 HLA-C03:03 HLA-C08:01 |
| | HLA-C01:03 HLA-C08:03 |
| VAKPPGTAF | HLA-B07:02 HLA-C03:04 HLA-C02:10 HLA-B15:03 HLA-C04:01 |
| | HLA-B35:03 HLA-B35:01 HLA-B15:01 HLA-B55:01 HLA-B15:18 |
| | HLA-B35:02 HLA-B07:05 HLA-B35:05 HLA-B42:01 HLA-B46:01 |
| | HLA-B39:10 HLA-B67:01 HLA-B57:03 HLA-B56:04 HLA-B15:02 |
| | HLA-B58:02 HLA-B15:17 HLA-B81:01 HLA-B15:10 HLA-B15:05 |
| | HLA-B42:02 HLA-B14:03 HLA-B35:08 HLA-B15:25 HLA-B15:08 |
| | HLA-B15:16 HLA-B15:07 HLA-B15:11 HLA-B78:01 HLA-B82:01 |
| | HLA-B55:04 HLA-B15:12 HLA-B15:13 HLA-B57:02 HLA-B07:04 |
| | HLA-B15:20 HLA-B15:30 HLA-B15:06 HLA-B15:27 HLA-B82:02 |
| | HLA-B15:31 HLA-B15:46 HLA-B15:58 HLA-B57:04 HLA-C06:02 |
| | HLA-C07:06 HLA-C07:02 HLA-C12:03 HLA-C07:01 HLA-C14:02 |
| | HLA-C01:02 HLA-C05:01 HLA-C03:03 HLA-C02:02 HLA-C16:01 |
| | HLA-C08:02 HLA-C07:18 HLA-C15:05 HLA-C04:07 HLA-C03:02 |
| | HLA-C16:02 HLA-C04:06 HLA-C08:01 HLA-C12:02 HLA-C08:04 |
| | HLA-C16:04 HLA-C14:03 HLA-C04:03 HLA-C01:03 HLA-C04:04 |
| | HLA-C15:04 HLA-C08:03 HLA-C07:17 HLA-C07:05 |
| VLMDEGAVLTL | HLA-A02:01 HLA-A02:02 HLA-A02:06 HLA-A02:03 HLA-A02:07 |
| | HLA-A02:14 HLA-A02:17 HLA-A02:04 HLA-A02:10 HLA-B15:30 |
| | HLA-B15:58 HLA-C05:01 HLA-C04:03 |
| VSDQQNGTY | HLA-C02:10 HLA-A01:01 HLA-A30:02 HLA-A01:03 HLA-A36:01 |
| | HLA-A26:08 HLA-A01:02 HLA-A80:01 HLA-B35:01 HLA-B15:18 |
| | HLA-B35:05 HLA-B15:17 HLA-B35:08 HLA-C05:01 HLA-C02:02 |
| | HLA-C08:02 HLA-C16:02 HLA-C04:06 HLA-C04:03 HLA-C01:03 |
| | HLA-C15:04 |
| VTLSTYFHV | HLA-A02:01 HLA-A68:02 HLA-A24:02 HLA-A23:01 HLA-A30:01 |
| | HLA-A30:04 HLA-A02:06 HLA-A32:01 HLA-A02:05 HLA-A24:07 |
| | HLA-A02:07 HLA-A02:14 HLA-A02:17 HLA-A02:04 HLA-A24:20 |
| | HLA-A69:01 HLA-A02:10 HLA-B13:02 HLA-B52:01 HLA-B51:01 |
| | HLA-B58:01 HLA-B13:01 HLA-B57:01 HLA-B57:03 HLA-B58:02 |
| | HLA-B15:17 HLA-B51:02 HLA-B51:08 HLA-B51:07 HLA-B59:01 |
| | HLA-B15:16 HLA-B57:02 HLA-C15:02 HLA-C15:05 HLA-C17:01 |
| | HLA-C16:02 HLA-C04:06 HLA-C08:01 HLA-C15:04 HLA-C08:03 |
| YKRMKLDSY | HLA-B15:03 HLA-B15:18 HLA-B15:46 HLA-C06:02 HLA-C07:06 |
| | HLA-C07:02 HLA-C07:01 HLA-C07:18 HLA-C07:17 HLA-C07:05 |
| YLLNCHLLI | HLA-A02:01 HLA-C03:04 HLA-C02:10 HLA-C04:01 HLA-A29:02 |
| | HLA-A24:02 HLA-A23:01 HLA-A02:02 HLA-A02:06 HLA-A32:01 |
| | HLA-A29:01 HLA-A02:05 HLA-A24:07 HLA-A02:03 HLA-A02:07 |
| | HLA-A02:14 HLA-A02:17 HLA-A02:04 HLA-A24:20 HLA-A80:01 |
| | HLA-A69:01 HLA-A02:10 HLA-B13:02 HLA-B52:01 HLA-B48:01 |
| | HLA-B13:01 HLA-B51:08 HLA-B48:03 HLA-B51:07 HLA-B59:01 |
| | HLA-B39:02 HLA-C07:06 HLA-C07:02 HLA-C07:01 HLA-C14:02 |
| | HLA-C01:02 HLA-C05:01 HLA-C03:03 HLA-C02:02 HLA-C16:01 |
| | HLA-C07:04 HLA-C08:02 HLA-C15:02 HLA-C07:18 HLA-C15:05 |
| | HLA-C04:07 HLA-C17:01 HLA-C03:02 HLA-C16:02 HLA-C04:06 |
| | HLA-C08:01 HLA-C12:02 HLA-C08:04 HLA-C16:04 HLA-C14:03 |
| | HLA-C04:03 HLA-C01:03 HLA-C04:04 HLA-C15:04 HLA-C08:03 |
| | HLA-C07:17 HLA-C07:05 |
| YSDQKPPYSY | HLA-C02:10 HLA-A01:01 HLA-C04:01 HLA-A30:02 HLA-A29:02 |
| | HLA-A01:03 HLA-A30:04 HLA-A29:01 HLA-A36:01 HLA-A26:08 |
| | HLA-A01:02 HLA-A80:01 HLA-B35:01 HLA-B46:01 HLA-B15:17 |
| | HLA-B15:05 HLA-B35:08 HLA-B15:31 HLA-B57:04 HLA-C05:01 |
| | HLA-C02:02 HLA-C08:02 HLA-C04:07 HLA-C16:02 HLA-C04:03 |
| | HLA-C15:04 |
| YSIYPMRNL | HLA-C03:04 HLA-C02:10 HLA-A68:02 HLA-A25:01 HLA-A26:01 |
| | HLA-A32:01 HLA-A66:02 HLA-A66:01 HLA-A34:01 HLA-A26:08 |
| | HLA-A66:03 HLA-A69:01 HLA-A26:03 HLA-A26:02 HLA-A43:01 |
| | HLA-B58:01 HLA-B46:01 HLA-B14:02 HLA-B57:01 HLA-B57:03 |
| | HLA-B58:02 HLA-B15:17 HLA-B15:16 HLA-B15:13 HLA-B57:02 |
| | HLA-B15:30 HLA-B15:58 HLA-B57:04 HLA-C06:02 HLA-C07:06 |
| | HLA-C07:02 HLA-C12:03 HLA-C07:01 HLA-C14:02 HLA-C01:02 |
| | HLA-C05:01 HLA-C03:03 HLA-C02:02 HLA-C16:01 HLA-C07:04 |
| | HLA-C08:02 HLA-C15:02 HLA-C07:18 HLA-C15:05 HLA-C17:01 |
| | HLA-C03:02 HLA-C16:02 HLA-C04:06 HLA-C08:01 HLA-C12:02 |
| | HLA-C08:04 HLA-C16:04 HLA-C14:03 HLA-C04:03 HLA-C01:03 |
| | HLA-C04:04 HLA-C15:04 HLA-C08:03 HLA-C07:17 HLA-C07:05 |
| YTPFPSYGHY | HLA-C02:10 HLA-B15:03 HLA-A01:01 HLA-B53:01 HLA-A68:01 |
| | HLA-A25:01 HLA-A26:01 HLA-A30:02 HLA-A29:02 HLA-A01:03 |
| | HLA-A30:04 HLA-A29:01 HLA-A11:10 HLA-A36:01 HLA-A66:02 |
| | HLA-A66:01 HLA-A34:01 HLA-A26:08 HLA-A01:02 HLA-A34:02 |
| | HLA-A80:01 HLA-A66:03 HLA-A26:03 HLA-A11:03 HLA-A26:02 |
| | HLA-A43:01 HLA-B35:01 HLA-B15:01 HLA-B55:01 HLA-B51:01 |
| | HLA-B15:18 HLA-B35:02 HLA-B35:05 HLA-B46:01 HLA-B56:04 |
| | HLA-B15:02 HLA-B15:17 HLA-B51:02 HLA-B15:05 HLA-B35:08 |
| | HLA-B15:25 HLA-B15:08 HLA-B15:16 HLA-B15:07 HLA-B15:11 |
| | HLA-B78:01 HLA-B56:01 HLA-B15:12 HLA-B15:13 HLA-B15:20 |
| | HLA-B15:06 HLA-B15:27 HLA-B15:31 HLA-B15:46 HLA-C06:02 |
| | HLA-C12:03 HLA-C02:02 HLA-C03:02 HLA-C16:02 HLA-C12:02 |
| | HLA-C16:04 HLA-C15:04 HLA-C07:17 |

It was found that, within most cancer types, the immune infiltration signature, derived herein using xCell (Aran et al., 2017b), was decreased in samples with high paMAP and saMAP counts or their source gene enrichment (FIG. 13C). In considering cell-autonomous mechanisms that could mediate escape from T-cell recognition, the expression of MHC-I molecules, whose downregulation leads to evasion from immune detection (Agudo et al., 2018; Castro et al., 2019), was first evaluated. A significant negative correlation was found between the number of paMAPs and saMAPs expressed per sample and the expression of genes involved in surface HLA expression (FIG. 7B). In addition, a negative association was found with the expression of genes encoding chemokines that recruit immune cells, including the BATF3⁺ DCs, which are important for cross-presenting tumor antigens (Spranger et al., 2017) (FIG. 7C). Furthermore, pathways strongly associated with paMAP and saMAP expression (FIGS. 5C and D), namely the activation of MYC and WNT-β-catenin signaling, the loss of function of P53, and the loss of PI3K pathway inhibitors, are known to inhibit T-cell activation and infiltration (Spranger and Gajewski, 2018). Accordingly, the number of paMAPs and saMAPs showed a strong positive correlation with the expression of CDK4 and CDK6 in nearly all TCGA cancer types (FIG. 7D). Importantly, WNT-β-catenin and TGF-β signaling, and CDK4/6, regulate cancer cell programs that promote T-cell exclusion and immune evasion in breast cancer and melanoma (Bagati et al., 2021; Goel et al., 2017; Jerby-Arnon et al., 2018; Spranger et al., 2015). Lastly, the stemness signature showed a positive pan-cancer correlation with the immunosuppressive genes PVR (CD155) and CD276 (B7-H3) (FIG. 13D). Collectively, these data suggest that cancers with high numbers of paMAPs and saMAPs employ multiple resistance mechanisms as a shield from immune detection and destruction. In conclusion, the data shows that an increased immune evasion and repression program may hinder immune recognition of paMAPs and saMAPs, which are found in poorly differentiated advanced cancers. Furthermore, they suggest that WNT-β-catenin, TGF-β, and CDK4/6 inhibitors, currently used to treat several types of cancer (Álvarez-Fernández and Malumbres, 2020; Hinze et al., 2020; Huang et al., 2021), could potentially enhance immune recognition of tumor cells expressing paMAPs and saMAPs.

Although the present invention has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, “an” and “the” include corresponding plural references unless the context clearly dictates otherwise.

### REFERENCES

Agudo, J., Park, E. S., Rose, S. A., Alibo, E., Sweeney, R., Dhainaut, M., Kobayashi, K. S., Sachidanandam, R., Baccarini, A., Merad, M., et al. (2018). Quiescent Tissue Stem Cells Evade Immune Surveillance. Immunity 48, 271-285.
Åkesson, E., Wolmer-Solberg, N., Cederarv, M., Falci, S., and Odeberg, J. (2009). Human neural stem cells and astrocytes, but not neurons, suppress an allogeneic lymphocyte response. Stem Cell Res. 2, 56-67.
Álvarez-Fernández, M., and Malumbres, M. (2020). Mechanisms of Sensitivity and Resistance to CDK4/6 Inhibition. Cancer Cell 37, 514-529.
Anderson, K. G., Stromnes, I. M., and Greenberg, P. D. (2017). Obstacles Posed by the Tumor Microenvironment to T cell Activity: A Case for Synergistic Therapies. Cancer Cell 31, 311-325.
Apavaloaei, A., Hardy, M., Thibault, P., and Perreault, C. (2020). The Origin and Immune Recognition of Tumor-Specific Antigens. Cancers (Basel). 12, 2607.
Aran, D., Sirota, M., and Butte, A. J. (2015). Systematic pan-cancer analysis of tumour purity. Nat. Commun. 6, 1-12.
Aran, D., Camarda, R., Odegaard, J., Paik, H., Oskotsky, B., Krings, G., Goga, A., Sirota, M., and Butte, A. J. (2017a). Comprehensive analysis of normal adjacent to tumor transcriptomes. Nat. Commun. 8, 1-13.
Aran, D., Hu, Z., and Butte, A. J. (2017b). xCell: Digitally portraying the tissue cellular heterogeneity landscape. Genome Biol. 18, 1-14.
Babarinde, I., Ma, G., Li, Y., Deng, B., Luo, Z., Liu, H., Abdul, M. M., Ward, C., Chen, M., Fu, X., et al. (2020). Transposable Element-Gene Splicing Modulates the Transcriptional Landscape of Human Pluripotent Stem Cells. BioRxiv.
Bagati, A., Kumar, S., Jiang, P., Pyrdol, J., Zou, A. E., Godicelj, A., Mathewson, N. D., Cartwright, A. N. R., Cejas, P., Brown, M., et al. (2021). Integrin avβ6-TGFβ-SOX4 Pathway Drives Immune Evasion in Triple-Negative Breast Cancer. Cancer Cell 39, 54-67.e9.
Barbie, D. A., Tamayo, P., Boehm, J. S., Kim, S. Y., Moody, S. E., Dunn, I. F., Schinzel, A. C., Sandy, P., Meylan, E., Scholl, C., et al. (2009). Systematic RNA interference reveals that oncogenic KRAS-driven cancers require TBK1. Nature 462, 108-112.
Bassani-Sternberg, M., Pletscher-Frankild, S., Jensen, L. J., and Mann, M. (2015). Mass Spectrometry of Human Leukocyte Antigen Class I Peptidomes Reveals Strong Effects of Protein Abundance and Turnover on Antigen Presentation. Mol. Cell. Proteomics 14, 658-673.
Batlle, E., and Clevers, H. (2017). Cancer stem cells revisited. Nat. Med. 23, 1124-1134.
Ben-Porath, I., Thomson, M. W., Carey, V. J., Ge, R., Bell, G. W., Regev, A., and Weinberg, R. A. (2008). An embryonic stem cell-like gene expression signature in poorly differentiated aggressive human tumors. Nat. Genet. 40, 499-507.
Benhammadi, M., Mathé, J., Dumont-Lagacé, M., Kobayashi, K. S., Gaboury, L., Brochu, S., and Perreault, C. (2020). IFN-λ Enhances Constitutive Expression of MHC Class I Molecules on Thymic Epithelial Cells. J. Immunol. 205, 1268-1280.
Bouwmeester, R., Gabriels, R., Hulstaert, N., Martens, L., and Degroeve, S. (2020). DeepLC can predict retention times for peptides that carry as-yet unseen modifications. BioRxiv.
Bray, N. L., Pimentel, H., Melsted, P., and Pachter, L. (2016). Near-optimal probabilistic RNA-seq quantification. Nat. Biotechnol. 34, 525-527.
Brewer, B. G., Mitchell, R. A., Harandi, A., and Eaton, J. W. (2009). Embryonic vaccines against cancer: An early history. Exp. Mol. Pathol. 86, 192-197.
Caron, E., Vincent, K., Fortier, M.-H., Laverdure, J.-P., Bramoullé, A., Hardy, M.-P., Voisin, G., Roux, P. P., Lemieux, S., Thibault, P., et al. (2011). The MHC I immunopeptidome conveys to the cell surface an integrative view of cellular regulation. Mol. Syst. Biol. 7, 1-15.
Castro, A., Ozturk, K., Pyke, R. M., Xian, S., Zanetti, M., and Carter, H. (2019). Elevated neoantigen levels in tumors with somatic mutations in the HLA-A, HLA-B, HLA-C and B2M genes. BMC Med. Genomics 12, 1-13.
Chia, N.Y., Chan, Y. S., Feng, B., Lu, X., Orlov, Y. L., Moreau, D., Kumar, P., Yang, L., Jiang, J., Lau, M. S., et al. (2010). A genome-wide RNAi screen reveals determinants of human embryonic stem cell identity. Nature 468, 316-320.
Chong, C., Müller, M., Pak, H. S., Harnett, D., Huber, F., Grun, D., Leleu, M., Auger, A., Arnaud, M., Stevenson, B. J., et al. (2020). Integrated proteogenomic deep sequencing and analytics accurately identify non-canonical peptides in tumor immunopeptidomes. Nat. Commun. 11, 1293.
Churko, J. M., Lee, J., Ameen, M., Gu, M., Venkatasubramanian, M., Diecke, S., Sallam, K., Im, H., Wang, G., Gold, J. D., et al. (2017). Transcriptomic and epigenomic differences in human induced pluripotent stem cells generated from six reprogramming methods. Nat. Biomed. Eng. 1, 826-837.
Colaprico, A., Silva, T. C., Olsen, C., Garofano, L., Cava, C., Garolini, D., Sabedot, T. S., Malta, T. M., Pagnotta, S. M., Castiglioni, I., et al. (2016). TCGAbiolinks: An R/Bioconductor package for integrative analysis of TCGA data. Nucleic Acids Res. 44, e71.
Coulie, P. G., Van Den Eynde, B. J., Van Der Bruggen, P., and Boon, T. (2014). Tumour antigens recognized by T lymphocytes: At the core of cancer immunotherapy. Nat. Rev. Cancer 14, 135-146.
Courcelles, M., Durette, C., Daouda, T., Laverdure, J.-P., Vincent, K., Lemieux, S., Perreault, C., and Thibault, P. (2020). MAPDP: A Cloud-Based Computational Platform for Immunopeptidomics Analyses. J. Proteome Res. 19, 1873-1881.
Dai, Z., Ramesh, V., and Locasale, J. W. (2020). The evolving metabolic landscape of chromatin biology and epigenetics. Nat. Rev. Genet. 21, 737-753.
Danilova, L., Anagnostou, V., Caushi, J. X., Sidhom, J. W., Guo, H., Chan, H. Y., Suri, P., Tam, A., Zhang, J., Asmar, M. El, et al. (2018). The mutation-associated neoantigen functional expansion of specific T cells (MANAFEST) assay: A sensitive platform for monitoring antitumor immunity. Cancer Immunol. Res. 6, 888-899.
Daouda, T., Perreault, C., and Lemieux, S. (2016). pyGeno: A Python package for precision medicine and proteogenomics. F1000 Res. 5, 381.
Dardenne, E., Beltran, H., Benelli, M., Gayvert, K., Berger, A., Puca, L., Cyrta, J., Sboner, A., Noorzad, Z., MacDonald, T., et al. (2016). N-Myc Induces an EZH2-Mediated Transcriptional Program Driving Neuroendocrine Prostate Cancer. Cancer Cell 30, 563-577.
Das, B., Pal, B., Bhuyan, R., Li, H., Sarma, A., Gayan, S., Talukdar, J., Sandhya, S., Bhuyan, S., Gogoi, G., et al. (2019). MYC Regulates the HIF2a Stemness Pathway via Nanog and Sox2 to Maintain Self-Renewal in Cancer Stem Cells versus Non-Stem Cancer Cells. Cancer Res. 79, 4015-4025.
Dobin, A., Davis, C. A., Schlesinger, F., Drenkow, J., Zaleski, C., Jha, S., Batut, P., Chaisson, M., and Gingeras, T. R. (2013). STAR: Ultrafast universal RNA-seq aligner. Bioinformatics 29, 15-21.
Duffour, M. T., Chaux, P., Lurquin, C., Cornelis, G., Boon, T., and Van Der Bruggen, P. (1999). A MAGE-A4 peptide presented by HLA-A2 is recognized by cytolytic T lymphocytes. Eur. J. Immunol. 29, 3329-3337.
Ehx, G., and Perreault, C. (2019). Discovery and characterization of actionable tumor antigens. Genome Med. 11, 10-12.
Ehx, G., Larouche, J.-D., Durette, C., Laverdure, J.-P., Hesnard, L., Vincent, K., Hardy, M.-P., Theriault, C., Rulleau, C., Lanoix, J., et al. (2021). Atypical acute myeloid leukemia-specific transcripts generate shared and immunogenic MHC class-I-associated epitopes Article Atypical acute myeloid leukemia-specific transcripts generate shared and immunogenic MHC class-I-associated epitopes. Immunity 54, 737-752.
Ellrott, K., Bailey, M. H., Saksena, G., Covington, K. R., Kandoth, C., Stewart, C., Hess, J., Ma, S., Chiotti, K. E., McLellan, M. D., et al. (2018). Scalable Open Science Approach for Mutation Calling of Tumor Exomes Using Multiple Genomic Pipelines. Cell Syst. 6, 271-281.e7.
Erhard, F., Dölken, L., Schilling, B., and Schlosser, A. (2020). Identification of the Cryptic HLA-I Immunopeptidome. Cancer Immunol. Res. 8, 1018-1026.
Fagnocchi, L., and Zippo, A. (2017). Multiple roles of MYC in integrating regulatory networks of pluripotent stem cells. Front. Cell Dev. Biol. 5, 1-19.
Fort, A., Hashimoto, K., Yamada, D., Salimullah, M., Keya, C. A., Saxena, A., Bonetti, A., Voineagu, I., Bertin, N., Kratz, A., et al. (2014). Deep transcriptome profiling of mammalian stem cells supports a regulatory role for retrotransposons in pluripotency maintenance. Nat. Genet. 46, 558-566.
Friedli, M., Turelli, P., Kapopoulou, A., Rauwel, B., Castro-Diaz, N., Rowe, H. M., Ecco, G., Unzu, C., Planet, E., Lombardo, A., et al. (2014). Loss of transcriptional control over endogenous retroelements during reprogramming to pluripotency. Genome Res. 24, 1251-1259.
Garrison, E., and Marth, G. (2012). Haplotype-based variant detection from short-read sequencing. ArXiv: Genomics.
Goel, S., Decristo, M. J., Watt, A. C., Brinjones, H., Sceneay, J., Li, B. B., Khan, N., Ubellacker, J. M., Xie, S., Metzger-Filho, O., et al. (2017). CDK4/6 inhibition triggers anti-tumour immunity. Nature 548, 471-475.
Goldman, M. J., Craft, B., Hastie, M., Repecka, K., McDade, F., Kamath, A., Banerjee, A., Luo, Y., Rogers, D., Brooks, A. N., et al. (2020). Visualizing and interpreting cancer genomics data via the Xena platform. Nat. Biotechnol. 38, 675-678.
Haen, S. P., Löffler, M. W., Rammensee, H. G., and Brossart, P. (2020). Towards new horizons: characterization, classification and implications of the tumour antigenic repertoire. Nat. Rev. Clin. Oncol. 17, 595-610.
Hanzelmann, S., Castelo, R., and Guinney, J. (2013). GSVA: Gene set variation analysis for microarray and RNA-Seq data. BMC Bioinformatics 14, 7.
Heiden, M. G. V., Cantley, L. C., and Thompson, C. B. (2009). Understanding the Warburg Effect: The Metabolic Requirements of Cell Proliferation. Science 324, 1029-1033.
Hernandez, C., Wang, Z., Ramazanov, B., Tang, Y., Mehta, S., Dambrot, C., Lee, Y. W., Tessema, K., Kumar, I., Astudillo, M., et al. (2018). Dppa2/4 Facilitate Epigenetic Remodeling during Reprogramming to Pluripotency. Cell Stem Cell 23, 396-411.e8.
Hinze, L., Labrosse, R., Degar, J., Han, T., Schatoff, E. M., Schreek, S., Karim, S., McGuckin, C., Sacher, J. R., Wagner, F., et al. (2020). Exploiting the Therapeutic Interaction of WNT Pathway Activation and Asparaginase for Colorectal Cancer Therapy. Cancer Discov. 10, 1690-1705.
Hong, S.-H., Lee, J.-H., Lee, J. B., Ji, J., and Bhatia, M. (2011). ID1 and ID3 represent conserved negative regulators of human embryonic and induced pluripotent stem cell hematopoiesis. J. Cell Sci. 124, 1445-1452.
Huang, C.-Y., Chung, C.-L., Hu, T.-H., Chen, J.-J., Liu, P.-F., and Chen, C.-L. (2021). Recent progress in TGF-β inhibitors for cancer therapy. Biomed. Pharmacother. 134.
Huang, L.-Q., Brasseur, F., Serrano, A., De Plaen, E., Bruggen, P. van der, Boon, T., and Pel, A. Van (1991). Cytolytic T Lymphocytes Recognize an Antigen Encoded by MAGE-A10 on a Human Melanoma. J. Immunol. 162, 6849-6854.
Humeau, J., Sauvat, A., Cerrato, G., Xie, W., Loos, F., Iannantuoni, F., Bezu, L., Levesque, S., Paillet, J., Pol, J., et al. (2020). Inhibition of transcription by dactinomycin reveals a new characteristic of immunogenic cell stress. EMBO Mol. Med. 12, el 1622.
Ishak, C. A., and De Carvalho, D. D. (2020). Reactivation of Endogenous Retroelements in Cancer Development and Therapy. Annu. Rev. Cancer Biol. 4, 159-176.
Izadyar, F., Wong, J., Maki, C., Pacchiarotti, J., Ramos, T., Howerton, K., Yuen, C., Greilach, S., Zhao, H. H., Chow, M., et al. (2011). Identification and characterization of repopulating spermatogonial stem cells from the adult human testis. Hum. Reprod. 26, 1296-1306.
Janku, F., Yap, T. A., and Meric-Bernstam, F. (2018). Targeting the PI3K pathway in cancer: Are we making headway? Nat. Rev. Clin. Oncol. 15, 273-291.
Jerby-Arnon, L., Shah, P., Cuoco, M. S., Rodman, C., Su, M. J., Melms, J. C., Leeson, R., Kanodia, A., Mei, S., Lin, J. R., et al. (2018). A Cancer Cell Program Promotes T Cell Exclusion and Resistance to Checkpoint Blockade. Cell 175, 984-997.e24.
Jia, Z. C., Ni, B., Huang, Z.-M., Tian, Y., Tang, J., Wang, J.-X., Fu, X.-L., and Wu, Y.-Z. (2010). Identification of two novel HLA-A*0201-restricted CTL epitopes derived from MAGE-A4. Clin. Dev. Immunol. 2010, 567594.
Jurtz, V., Paul, S., Andreatta, M., Marcatili, P., Peters, B., and Nielsen, M. (2017). NetMHCpan-4.0: Improved Peptide-MHC Class I Interaction Predictions Integrating Eluted Ligand and Peptide Binding Affinity Data. J. Immunol. 199, 3360-3368.
Kazantseva, J., Sadam, H., Neuman, T., and Palm, K. (2016). Targeted alternative splicing of TAF4: A new strategy for cell reprogramming. Sci. Rep. 6, 1-11.
Kelley, D., and Rinn, J. (2012). Transposable elements reveal a stem cell-specific class of long noncoding RNAs. Genome Biol. 13, R107.
Klawitter, S., Fuchs, N. V., Upton, K. R., Munoz-Lopez, M., Shukla, R., Wang, J., Garcia-Canadas, M., Lopez-Ruiz, C., Gerhardt, D. J., Sebe, A., et al. (2016). Reprogramming triggers endogenous L1 and Alu retrotransposition in human induced pluripotent stem cells. Nat. Commun. 7, 10286.
Kooreman, N. G., Kim, Y., de Almeida, P. E., Termglinchan, V., Diecke, S., Shao, N.Y., Wei, T. T., Yi, H., Dey, D., Nelakanti, R., et al. (2018). Autologous iPSC-Based Vaccines Elicit Anti-tumor Responses In Vivo. Cell Stem Cell 22, 501-513.
Kroemer, G., and Pouyssegur, J. (2008). Tumor Cell Metabolism: Cancer's Achilles' Heel. Cancer Cell 13, 472-482.
Krokhin, O. V (2006). Sequence-specific retention calculator. Algorithm for peptide retention prediction in ion-pair RP-HPLC: application to 300- and 100-A pore size C18 sorbents. Anal. Chem. 78, 7785-7795.
Lamoliatte, F., McManus, F. P., Maarifi, G., Chelbi-Alix, M. K., and Thibault, P. (2017). Uncovering the SUMOylation and ubiquitylation crosstalk in human cells using sequential peptide immunopurification. Nat. Commun. 8, 14109.
Laumont, C. M., Vincent, K., Hesnard, L., Audemard, É., Bonneil, É., Laverdure, J.-P., Gendron, P., Courcelles, M., Hardy, M.-P., Côté, C., et al. (2018). Noncoding regions are the main source of targetable tumor-specific antigens. Sci. Transl. Med. 10, 1-11.
Li, L., Baroja, M. L., Majumdar, A., Chadwick, K., Rouleau, A., Gallacher, L., Ferber, I., Lebkowski, J., Martin, T., Madrenas, J., et al. (2004). Human Embryonic Stem Cells Possess Immune-Privileged Properties. Stem Cells 22, 448-456.
Liberzon, A., Birger, C., Thorvaldsdóttir, H., Ghandi, M., Mesirov, J. P., and Tamayo, P. (2015). The Molecular Signatures Database Hallmark Gene Set Collection. Cell Syst. 1, 417-425.
Lin, T., and Lin, Y. (2017). P53 Switches Off Pluripotency on Differentiation. Stem Cell Res. Ther. 8, 44.
Liu, J., Lichtenberg, T., Hoadley, K. A., Poisson, L. M., Lazar, A. J., Cherniack, A. D., Kovatich, A. J., Benz, C. C., Levine, D. A., Lee, A. V., et al. (2018). An Integrated TCGA Pan-Cancer Clinical Data Resource to Drive High-Quality Survival Outcome Analytics. Cell 173, 400-416.e11.
Löffler, M. W., Mohr, C., Bichmann, L., Freudenmann, L. K., Walzer, M., Schroeder, C. M., Trautwein, N., Hilke, F. J., Zinser, R. S., Muhlenbruch, L., et al. (2019). Multi-omics discovery of exome-derived neoantigens in hepatocellular carcinoma. Genome Med. 11, 1-16.
Loizou, E., Banito, A., Livshits, G., Ho, Y. J., Koche, R. P., Sánchez-Rivera, F. J., Mayle, A., Chen, C. C., Kinalis, S., Bagger, F. O., et al. (2019). A gain-of-function p53-mutant oncogene promotes cell fate plasticity and myeloid leukemia through the pluripotency factor foxh1. Cancer Discov. 9, 962-979.
Lonsdale, J., Thomas, J., Salvatore, M., Phillips, R., Lo, E., Shad, S., Hasz, R., Walters, G., Garcia, F., Young, N., et al. (2013). The Genotype-Tissue Expression (GTEx) project. Nat. Genet. 45, 580-585.
Lu, X., Sachs, F., Ramsay, L. A., Jacques, P. É., Göke, J., Bourque, G., and Ng, H. H. (2014). The retrovirus HERVH is a long noncoding RNA required for human embryonic stem cell identity. Nat. Struct. Mol. Biol. 21, 423-425.
Ma, J., Zhao, J., Lu, J., Wang, P., Feng, H., Zong, Y., Ou, B., Zheng, M., and Lu, A. (2016). Cadherin-12 enhances proliferation in colorectal cancer cells and increases progression by promoting EMT. Tumor Biol. 37, 9077-9088.
Madsen, R. R. (2020). PI3K in stemness regulation: From development to cancer. Biochem. Soc. Trans. 48, 301-315.
Malta, T. M., Sokolov, A., Gentles, A. J., Burzykowski, T., Poisson, L., Weinstein, J. N., Kamińska, B., Huelsken, J., Omberg, L., Gevaert, O., et al. (2018). Machine Learning Identifies Stemness Features Associated with Oncogenic Dedifferentiation. Cell 173, 338-354.e15.
Margais, G., and Kingsford, C. (2011). A fast, lock-free approach for efficient parallel counting of occurrences of k-mers. Bioinformatics 27, 764-770.
McLaughlin, R. N., Young, J. M., Yang, L., Neme, R., Wichman, H. A., and Malik, H. S. (2014). Positive Selection and Multiple Losses of the LINE-1-Derived L1TD1 Gene in Mammals Suggest a Dual Role in Genome Defense and Pluripotency. PLoS Genet. 10.
Merkle, F. T., Ghosh, S., Kamitaki, N., Mitchell, J., Avior, Y., Mello, C., Kashin, S., Mekhoubad, S., Ilic, D., Charlton, M., et al. (2017). Human pluripotent stem cells recurrently acquire and expand dominant negative P53 mutations. Nature 545, 229-233.
Mi, H., Ebert, D., Muruganujan, A., Mills, C., Albou, L.-P., Tremayne, M., and Thomas, P. D. (2021). PANTHER version 16: a revised family classification, tree-based classification tool, enhancer regions and extensive API. Nucleic Acids Res. 49, D394-D403.
Miranda, A., Hamilton, P. T., Zhang, A. W., Pattnaik, S., Becht, E., Mezheyeuski, A., Bruun, J., Micke, P., de Reynies, A., and Nelson, B. H. (2019). Cancer stemness, intratumoral heterogeneity, and immune response across cancers. Proc. Natl. Acad. Sci. U.S.A 116, 9020-9029.
Närvä, E., Rahkonen, N., Emani, M. R., Lund, R., Pursiheimo, J. P., Nästi, J., Autio, R., Rasool, O., Denessiouk, K., Lähdesmäki, H., et al. (2012). RNA-binding protein L1TD1 interacts with LIN28 via RNA and is required for human embryonic stem cell self-renewal and cancer cell proliferation. Stem Cells 30, 452-460.
Nicola, M. Di, Carlo-Stella, C., Magni, M., Milanesi, M., Longoni, P. D., Matteucci, P., Grisanti, S., and Gianni, A. M. (2002). Human bone marrow stromal cells suppress T-lymphocyte proliferation induced by cellular or nonspecific mitogenic stimuli. Blood 99, 3838-3843.
Ochsenbein, A. F. (2005). Immunological ignorance of solid tumors. Springer Semin. Immunopathol. 27, 19-35.
Ogishi, M., and Yotsuyanagi, H. (2019). Quantitative prediction of the landscape of T cell epitope immunogenicity in sequence space. Front. Immunol. 10, 827.
Pearson, H., Thibault, P., Perreault, C., Pearson, H., Daouda, T., Granados, D. P., Durette, C., Bonneil, E., Courcelles, M., Rodenbrock, A., et al. (2016). MHC class I-associated peptides derive from selective regions of the human genome. J. Clin. Invest. 126, 4690-4701.
Pelullo, M., Zema, S., Nardozza, F., Checquolo, S., Screpanti, I., and Bellavia, D. (2019). Wnt, Notch, and TGF-β pathways impinge on hedgehog signaling complexity: An open window on cancer. Front. Genet. 10, 1-16.
Petroni, G., Buqué, A., Zitvogel, L., Kroemer, G., and Galluzzi, L. (2021). Immunomodulation by targeted anticancer agents. Cancer Cell 39, 310-345.
Poli, V., Fagnocchi, L., Fasciani, A., Cherubini, A., Mazzoleni, S., Ferrillo, S., Miluzio, A., Gaudioso, G., Vaira, V., Turdo, A., et al. (2018). MYC-driven epigenetic reprogramming favors the onset of tumorigenesis by inducing a stem cell-like state. Nat. Commun. 9, 1024.
Prensner, J. R., Iyer, M. K., Balbin, O. A., Dhanasekaran, S. M., Cao, Q., Brenner, J. C., Laxman, B., Asangani, I. A., Grasso, C. S., Kominsky, H. D., et al. (2011). Transcriptome sequencing across a prostate cancer cohort identifies PCAT-1, an unannotated lincRNA implicated in disease progression. Nat. Biotechnol. 29, 742-749.
Reinhard, K., Rengstl, B., Oehm, P., Michel, K., Billmeier, A., Hayduk, N., Klein, O., Kuna, K., Ouchan, Y., Wöll, S., et al. (2020). An RNA vaccine drives expansion and efficacy of claudin-CAR-T cells against solid tumors. Science 367, 446-453.
Van Rhenen, A., Feller, N., Kelder, A., Westra, A. H., Rombouts, E., Zweegman, S., Van Der Pol, M. A., Waisfisz, Q., Ossenkoppele, G. J., and Schuurhuis, G. J. (2005). High stem cell frequency in acute myeloid leukemia at diagnosis predicts high minimal residual disease and poor survival. Clin. Cancer Res. 11, 6520-6527.
Robinson, J. T., Thorvaldsdóttir, H., Winckler, W., Guttman, M., Lander, E. S., Getz, G., and Mesirov, J. P. (2011). Integrative genomics viewer. Nat. Biotechnol. 29, 24-26.
Ruiz Cuevas, M. V., Hardy, M. P., Hollý, J., Bonneil, É., Durette, C., Courcelles, M., Lanoix, J., Côté, C., Staudt, L. M., Lemieux, S., et al. (2021). Most non-canonical proteins uniquely populate the proteome or immunopeptidome. Cell Rep. 34, 108815.
Sahin, U., Oehm, P., Derhovanessian, E., Jabulowsky, R. A., Vormehr, M., Gold, M., Maurus, D., Schwarck-Kokarakis, D., Kuhn, A. N., Omokoko, T., et al. (2020). An RNA vaccine drives immunity in checkpoint-inhibitor-treated melanoma. Nature 585, 107-112.
Salmon, H., Idoyaga, J., Rahman, A., Leboeuf, M., Remark, R., Jordan, S., Casanova-Acebes, M., Khudoynazarova, M., Agudo, J., Tung, N., et al. (2016). Expansion and Activation of CD103+ Dendritic Cell Progenitors at the Tumor Site Enhances Tumor Responses to Therapeutic PD-L1 and BRAF Inhibition. Immunity 44, 924-938.
Sanchez-Vega, F., Mina, M., Armenia, J., Chatila, W. K., Luna, A., La, K. C., Dimitriadoy, S., Liu, D. L., Kantheti, H. S., Saghafinia, S., et al. (2018). Oncogenic Signaling Pathways in The Cancer Genome Atlas. Cell 173, 321-337.
Schumacher, T. N., Scheper, W., and Kvistborg, P. (2019). Cancer Neoantigens. Annu. Rev. Immunol. 37, 173-200.
Schuster, H., Peper, J. K., Bösmüller, H. C., Röhle, K., Backert, L., Bilich, T., Ney, B., Löffler, M. W., Kowalewski, D. J., Trautwein, N., et al. (2017). The immunopeptidomic landscape of ovarian carcinomas. Proc. Natl. Acad. Sci. U.S.A 114, E9942-E9951.
Smith, B. A., Balanis, N. G., Nanjundiah, A., Sheu, K. M., Tsai, B. L., Zhang, Q., Park, J. W., Thompson, M., Huang, J., Witte, O. N., et al. (2018). A Human Adult Stem Cell Signature Marks Aggressive Variants across Epithelial Cancers. Cell Rep. 24, 3353-3366.e5.
Spranger, S., and Gajewski, T. F. (2018). Impact of oncogenic pathways on evasion of antitumour immune responses. Nat. Rev. Cancer 18, 139-147.
Spranger, S., Bao, R., and Gajewski, T. F. (2015). Melanoma-intrinsic β-catenin signalling prevents anti-tumour immunity. Nature 523, 231-235.
Spranger, S., Dai, D., Horton, B., and Gajewski, T. F. (2017). Tumor-Residing Batf3 Dendritic Cells Are Required for Effector T Cell Trafficking and Adoptive T Cell Therapy. Cancer Cell 31, 711-723.e4.
Stewart, M. H., Bosse, M., Chadwick, K., Menendez, P., Bendall, S. C., and Bhatia, M. (2006). Clonal isolation of hESCs reveals heterogeneity within the pluripotent stem cell compartment. Nat. Methods 3, 807-815.
Stine, Z. E., Walton, Z. E., Altman, B. J., Hsieh, A. L., and Dang, C. V. (2015). MYC, metabolism, and cancer. Cancer Discov. 5, 1024-1039.
Suárez-Álvarez, B., Rodriguez, R. M., Calvanese, V., Blanco-Gelaz, M. A., Suhr, S. T., Ortega, F., Otero, J., Cibelli, J. B., Moore, H., Fraga, M. F., et al. (2010). Epigenetic mechanisms regulate MHC and antigen processing molecules in human embryonic and induced pluripotent stem cells. PLoS One 5, e10192.
Thorsson, V., Gibbs, D. L., Brown, S. D., Wolf, D., Bortone, D. S., Ou Yang, T. H., Porta-Pardo, E., Gao, G. F., Plaisier, C. L., Eddy, J. A., et al. (2018). The Immune Landscape of Cancer. Immunity 48, 812-830.e14.
Vasileiou, S., Lulla, P. D., Tzannou, I., Watanabe, A., Kuvalekar, M., Callejas, W. L., Bilgi, M., Wang, T., Wu, M. J., Kamble, R., et al. (2021). T-Cell Therapy for Lymphoma Using Nonengineered Multiantigen-Targeted T Cells Is Safe and Produces Durable Clinical Effects. J. Clin. Oncol. 39, 1415-1425.
Vogel, C., and Marcotte, E. M. (2009). Absolute abundance for the masses. Nat. Biotechnol. 27, 825-826.
Wang, J. F., She, L., Su, B. H., Ding, L. C., Zheng, F. F., Zheng, D. L., and Lu, Y. G. (2011). CDH12 promotes the invasion of salivary adenoid cystic carcinoma. Oncol. Rep. 26, 101-108.
Wang, L., Su, Y., Huang, C., Yin, Y., Zhu, J., Knupp, A., Chu, A., and Tang, Y. (2019). FOXH1 Is Regulated by NANOG and LIN28 for Early-stage Reprogramming. Sci. Rep. 9, 1-8.
Wang, Z.-X., Kueh, J. L. L., Teh, C. H.-L., Rossbach, M., Lim, L., Li, P., Wong, K.-Y., Lufkin, T., Robson, P., and Stanton, L. W. (2007). Zfp206 Is a Transcription Factor That Controls Pluripotency of Embryonic Stem Cells. Stem Cells 25, 2173-2182.
Wei, L. H., and Guo, J. U. (2020). Coding functions of “noncoding” RNAs. Science 367, 1074-1075.
Wu, T. D., and Nacu, S. (2010). Fast and SNP-tolerant detection of complex variants and splicing in short reads. Bioinformatics 26, 873-881.
Yaddanapudi, K., Mitchell, R. A., Putty, K., Willer, S., Sharma, R. K., Yan, J., Bodduluri, H., and Eaton, J. W. (2012). Vaccination with Embryonic Stem Cells Protects against Lung Cancer: Is a Broad-Spectrum Prophylactic Vaccine against Cancer Possible? PLoS One 7, e42289.
Yewdell, J. W. (2010). Designing CD8+ T cell vaccines: It's not rocket science (yet). Curr. Opin. Immunol. 22, 402-410.
Yu, H. B., Kunarso, G., Hong, F. H., and Stanton, L. W. (2009). Zfp206, Oct4, and Sox2 are integrated components of a transcriptional regulatory network in embryonic stem cells. J. Biol. Chem. 284, 31327-31335.
Zhang, H. Le, Wang, P., Lu, M. Z., Zhang, S. D., and Zheng, L. (2019). c-Myc maintains the self-renewal and chemoresistance properties of colon cancer stem cells. Oncol. Lett. 17, 4487-4493.
Zhang, J., Nuebel, E., Daley, G. Q., Koehler, C. M., and Teitell, M. A. (2012). Metabolic regulation in pluripotent stem cells during reprogramming and self-renewal. Cell Stem Cell 11, 589-595.
Zhang, J., Ratanasirintrawoot, S., Chandrasekaran, S., Wu, Z., Ficarro, S. B., Yu, C., Ross, C. A., Cacchiarelli, D., Xia, Q., Seligson, M., et al. (2016). LIN28 Regulates Stem Cell Metabolism and Conversion to Primed Pluripotency. Cell Stem Cell 19, 66-80.
Zhao, Q., Laverdure, J.-P., Lanoix, J., Durette, C., Côté, C., Bonneil, É., Laumont, C. M., Gendron, P., Vincent, K., Courcelles, M., et al. (2020). Proteogenomics Uncovers a Vast Repertoire of Shared Tumor-Specific Antigens in Ovarian Cancer. Cancer Immunol. Res. 8, 544-555.
Zhao, S., Zhu, W., Xue, S., and Han, D. (2014). Testicular defense systems: Immune privilege and innate immunity. Cell. Mol. Immunol. 11, 428-437.
Zheng, K., Wu, X., Kaestner, K. H., and Wang, P. J. (2009). The pluripotency factor LIN28 marks undifferentiated spermatogonia in mouse. BMC Dev. Biol. 9, 1-11.
Zhou, X., Guo, X., Chen, M., Xie, C., and Jiang, J. (2018). HIF-3a promotes metastatic phenotypes in pancreatic cancer by transcriptional regulation of the RhoC-ROCK1 signaling pathway. Mol. Cancer Res. 16, 124-134.
Zitvogel, L., Perreault, C., Finn, O. J., and Kroemer, G. (2021). Beneficial autoimmunity improves cancer prognosis. Nat. Rev. Clin. Oncol.

1. A cancer stem cell (CSC) tumor antigen peptide (TAP) comprising one of the following amino acid sequences:

| Peptide | SEQ | Peptide | SEQ |
|---|---|---|---|
| sequence | ID NO: | sequence | ID NO: |
| NTENYILWGY | 1 | ISMCDLVY | 21 |
| MKFGNQVSGLF | 2 | YKRMKLDSY | 22 |
| RLQHEPPHPV | 3 | QPLPEPLQLW | 23 |
| LPMWKALLF | 4 | IPMKIYLW) | 24 |
| RPARPPAGL | 5 | SQSSLMLYL | 25 |
| FAYPNQKVTF | 6 | ALYPQPPTV | 26 |
| GQAHPQGSF | 7 | YTPFPSYGHY | 27 |
| YSDQKPPYSY | 8 | FTEEDLHFVLY | 28 |
| KLAQIIRQV | 9 | GQITHNTSF | 29 |
| GEIKTFSDL | 10 | VTLSTYFHV | 30 |
| GKLDNTNEY | 11 | GQFDRPAGV | 31 |
| AKKKENITY | 12 | VSDQQNGTY | 32 |
| SAQGKPTYF | 13 | KHFDSPRGVAF | 33 |
| EEEIHSPTL | 14 | GEHLVSVTL | 34 |
| SKLRSTGQSF | 15 | YSIYPMRNL | 35 |
| NTLSESYIY | 16 | SQNSPIRY | 36 |
| QPLPQPLELW | 17 | FHSQNSPIRY | 37 |
| RTDTGKRVLY | 18 | VAKPPGTAF | 38 |
| LPSGETIAKW | 19 | SLLGSSEILEV | 39 |
| KLDSYIIPY | 20 | | |
| GKFQGLIEKF | 47 | ALLEGVNTVVV | 55 |
| KQMENDIQLY | 48 | SETHPPEVAL | 56 |
| ASTPASSEL | 49 | SPQEASGVRW | 57 |
| RLWNETVELF | 50 | YLLNCHLLI | 58 |
| FGDGKFSEV | 51 | GQFLVKSGY | 59 |
| SEVSADKLVAL | 52 | QTELNNSKQEY | 60 |
| AMYHALEKA | 53 | MAWNGILHL | 61 |
| FAYPNQKDF | 54 | HPLPGLILEW | 62 |

or a nucleic acid encoding said CSC TAP.

2. The CSC TAP or nucleic acid of claim 1, wherein said CSC TAP comprises one of the sequences defined in SEQ ID NO: 1-39.

3. The CSC TAP or nucleic acid of claim 1, wherein said CSC TAP (a) binds to an HLA-A*01:01 molecule and comprises the sequence of SEQ ID NO: 1, 8, 16, 20, 21, 27, 28, 32, 37 or 60; (b) binds to an HLA-A*02:01 molecule and comprises the sequence of SEQ ID NO: 3, 6, 26, 30, 31, 39, 53, 55 or 58; (c) binds to an HLA-B*07:02 molecule and comprises the sequence of SEQ ID NO: 5; (d) binds to an HLA-B*15:03 molecule and comprises the sequence of SEQ ID NO: 2, 7, 11, 12, 15, 22, 29, 36, 38, 47, 48, or 59; (e) binds to an HLA-B*40:01 molecule and comprises the sequence of SEQ ID NO: 10, 25, 34, 52 or 56; (f) binds to an HLA-B*53:01 molecule and comprises the sequence of SEQ ID NO: 4, 17, 19, 23, 24 or 57; (g) binds to an HLA-C*02:10 molecule and comprises the sequence of SEQ ID NO: 6, 54 or 61; (h) binds to an HLA-C*03:04 molecule and comprises the sequence of SEQ ID NO: 6, 35, 49 or 51; or (i) binds to an HLA-C*04:01 molecule and comprises the sequence of SEQ ID NO: 13, 33 or 50.

4-11. (canceled)

12. The CSC TAP or nucleic acid of claim 1, wherein said CSC TAP is encoded by a sequence located a non-protein coding region of the genome.

13. The CSC TAP or nucleic acid of claim 12, wherein said non-protein coding region of the genome is (a) an untranslated transcribed region (UTR); (b) an intron; (c) an intergenic region; or (d) a long non-coding RNAs.

14-16. (canceled)

17. The CSC TAP or nucleic acid of claim 1, which is an mRNA.

18. A combination comprising at least two of the CSC TAPs or nucleic acids defined in claim cl.

19-21. (canceled)

22. A lipid vesicle or particle comprising the CSC TAP or nucleic acid of claim 1.

23. The lipid vesicle or particle of claim 22, wherein the lipid vesicle is a lipid nanoparticle (LNP).

24. (canceled)

25. A composition comprising the CSC TAP or nucleic acid of claim 1, and a pharmaceutically acceptable carrier.

26. A vaccine comprising the CSC TAP or nucleic acid of claim 1, and an adjuvant.

27-30. (canceled)

31. An isolated cell expressing at its surface major histocompatibility complex (MHC) class I molecules comprising the CSC TAP of claim 1 in their peptide binding groove.

32-33. (canceled)

34. A T-cell receptor (TCR) that specifically recognizes MHC class I molecules expressed at the surface of the cell of claim 31.

35. An antibody or an antigen-binding fragment thereof that specifically binds to MHC class I molecules expressed at the surface of the cell of claim 31.

36-39. (canceled)

40. An isolated cell expressing at its cell surface the TCR of claim 34.

41. The isolated cell of claim 40, which is a CD8⁺ T lymphocyte.

42. A cell population comprising at least 0.5% of the isolated cell as defined in claim 40.

43. A method of treating cancer in a subject comprising administering to the subject an effective amount of:
(i) the CSC TAP or nucleic acid of claim 1;
(ii) a lipid vesicle or particle comprising the CSC TAP or nucleic acid of (i);
(iii) a composition comprising the CSC TAP or nucleic acid of (i) or the lipid vesicle or particle of (ii);
(iv) a vaccine comprising the CSC TAP or nucleic acid of (i), the lipid vesicle or particle of (ii), or the composition of (iii);
(v) a cell expressing at its surface major histocompatibility complex (MHC) class I molecules comprising the CSC TAP of (i);
(vi) a T-cell receptor (TCR) that specifically recognizes MHC class I molecules expressed at the surface of the cell of (v);
(vii) a cell or population of cells expressing at its surface the TCR of (vi), or
(viii) an antibody or antigen-binding fragment thereof that specifically binds to MHC class I molecules expressed at the surface of the cell of (v).

44. The method of claim 43, wherein the cancer is leukemia brain cancer, breast cancer, lung cancer, gastrointestinal cancer, liver cancer, ovarian cancer, pancreatic cancer, prostate cancer, skin cancer, head and neck cancer or myeloma.

45. The method of claim 43, further comprising administering at least one additional antitumor agent or therapy to the subject, wherein the at least one additional antitumor agent or therapy comprises a chemotherapeutic agent, immunotherapy, an immune checkpoint inhibitor, radiotherapy, surgery, an inhibitor of CDK4/6, an inhibitor of TGF-β, or an inhibitor of WNT-β-catenin.

46-57. (canceled)
